(19) United States
(12) Patent Application Publication
(10) Pub. No.: US 2010/0196390 A1
(43) Pub. Date: Aug. 5, 2010

(54) AGONIST ANTI-TRKB MONOCLONAL ANTIBODIES

(75) Inventors: Chia-Yang Lin, Palo Alto, CA (US); Javier Fernando Chaparro Riggers, San Mateo, CA (US); Ruslan Nikolaevich Grishanin, San Francisco, CA (US); Jennifer Renee Stratton, Belmont, CA (US); Wenwu Zhai, Redwood City, CA (US)

Correspondence Address: PFIZER INC, 10555 SCIENCE CENTER DRIVE, SAN DIEGO, CA, 92121, US

(73) Assignee: RINAT NEUROSCIENCE CORP.

(21) Appl. No.: 12/697,983

(22) Filed: Feb. 1, 2010

Related U.S. Application Data
(60) Provisional application No. 61/149,159, filed on Feb. 2, 2009.
(60) Provisional application No. 61/234,421, filed on Aug. 17, 2009.
(60) Provisional application No. 61/266,884, filed on Dec. 4, 2009.
(60) Provisional application No. 61/289,316, filed on Dec. 22, 2009.

(51) Int. Cl. A61K 39/395; C07K 16/00; C12N 5/00; C12N 15/13; A61P 43/00

(52) U.S. Cl. 424/146.1; 530/389.1; 435/325; 536/23.53; 424/158.1

(57) ABSTRACT

The present invention provides TrkB agonist antibodies. The invention further relates to therapeutic methods for use of these antibodies and antigen-binding portions thereof to improve nerve function, including treatment of peripheral neuropathies, such as Charcot-Marie-Tooth disease.

### RELATED APPLICATIONS

This application claims the priority benefit of the provisional patent applications U.S. Ser. Nos. 61/149,159 filed on Feb. 2, 2009; 61/234,421 filed on Aug. 17, 2009; 61/266,884 filed on Dec. 4, 2009; and 61/289,316 filed on Dec. 22, 2009, each of which is incorporated herein by reference in its entirety.

### REFERENCE TO SEQUENCE LISTING

This application is being filed electronically via EFS-Web and includes an electronically submitted sequence listing in .txt format. The .txt file contains a sequence listing entitled “PC33869ASeqList.txt” created on Jan. 26, 2010 and having a size of 181 KB. The sequence listing contained in this .txt file is part of the specification and is incorporated herein by reference in its entirety.

### FIELD OF THE INVENTION

The present invention relates to agonist anti-tropomyosin-receptor-kinase B (TrkB) monoclonal antibodies. More specifically, the invention relates to compositions comprising agonist anti-TrkB monoclonal antibodies, and methods of using these antibodies a medicament. The agonist anti-TrkB antibodies can be used, for example, therapeutically to improve nerve function and can be used in the prevention and/or treatment of cellular degeneration, including nerve cell damage associated with acute nervous cell system injury and chronic neurodegenerative diseases, including peripheral neuropathies.

### BACKGROUND OF THE INVENTION

Neurotrophins are a family of small, homodimeric proteins, which play a crucial role in the development and maintenance of the nervous system. Members of the neurotrophin family include nerve growth factor (NGF), brain-derived neurotrophic factor (BDNF), neurotrophin-3 (NT-3), neurotrophin-4/5 (NT-4/5), neurotrophin-6 (NT-6), and neurotrophin-7 (NT-7). Neurotrophins, similar to other polypeptide growth factors, affect their target cells through interactions with cell surface receptors. According to current knowledge, two kinds of transmembrane glycoproteins serve as receptors for neurotrophins. Neurotrophin-responsive neurons possess a common low molecular weight (65-80 kDa), low affinity receptor (LNGFR), also known as p75NTR or p75, which binds NGF, BDNF, NT-3 and NT-4/5 with a K_{D} of 2×10⁻⁹ M; and large molecular weight (130-150 kDa), high-affinity (K_{D} in the 10⁻¹¹ M range) receptors, which are members of the trk family of receptor tyrosine kinases. The identified members of the trk receptor family are TrkA, TrkB, and TrkC.

Each of the different trk receptors exhibits particular binding affinity for the different neurotrophins, although there is some overlap. TrkA is believed to bind not only NGF, but also NT-3 and NT-4/5 (but not BDNF). TrkB is believed to bind BDNF, NT-3, NT-4 and NT-4/5, but not NGF. Treatment with TrkB agonists has been shown to result in weight loss in mice and increased body weight in cynomolgus monkeys. See, U.S. Pat. Appl. Pub. Nos. 2005/0209148 and 2007/0248611, each of which is herein incorporated by reference in its entirety. In contrast to TrkA and TrkB, TrkC is believed to bind only NT-3 and not any of the other neurotrophins.

A number of studies have validated the trk receptors as therapeutic targets for brain repair. See, for example, Liu et al., J. Neurosci. 1999, 19:4370-4387; Menei et al., Eur. J. Neurosci. 1998, 10:607-621; and Kobayashi et al., J. Neurosci. 1997, 17:9583-9595. The trk receptors and their ligands have also been studied using X-ray crystallography to obtain three-dimensional structures of the ligand-receptor binding complexes. Wiesmann et al., Nature 1999, 401:184-188; Banfield et al., Structure (Camb) 2001, 9:1191-1199. These and other studies suggest that neurotrophin binding to the trk receptors induces dimerization of receptor monomers, resulting in an increase of the intrinsic tyrosine kinase activity of the receptors. This increased activity triggers, in turn, signaling cascades that are believed to be beneficial to neurons by promoting neuronal survival, axonal growth, and synaptic plasticity. Snider, Cell 1994, 77:627-638; Kaplan & Miller, Curr. Opin. Neurobiol. 2000, 10:381-391.

Charcot-Marie-Tooth disease (CMT) is one of the most common inherited neurological disorders, affecting approximately 1 in 2,500 people in the United States. See, Charcot-Marie-Tooth Disease Fact Sheet, published by the National Institute of Neurological Disorders and Stroke, 2007. CMT, also known as hereditary motor and sensory neuropathy (HMSN) or peroneal muscular atrophy, comprises a group of disorders that affect peripheral nerves. The peripheral nerves lie outside the brain and spinal cord and supply the muscles and sensory organs in the limbs. CMT is caused by mutations in genes that produce proteins involved in the structure and function of either the peripheral nerve axon or the myelin sheath. Although different proteins are abnormal in different forms of CMT disease, all of the mutations affect the normal function of the peripheral nerves. Consequently, these nerves slowly degenerate and lose the ability to communicate with their distant targets. The degeneration of motor nerves results in muscle weakness and atrophy in the extremities (arms, legs, hands, or feet), and in some cases the degeneration of sensory nerves results in a reduced ability to feel heat, cold, and pain.

There are many forms of CMT disease, including CMT1, CMT2, CMT3, CMT4, and CMTX. CMT1, caused by abnormalities in the myelin sheath, has three main types. CMT1A is an autosomal dominant disease resulting from a duplication of the gene on chromosome 17 that carries the instructions for producing the peripheral myelin protein-22 (PMP-22). The PMP-22 protein is a critical component of the myelin sheath. An overabundance of this gene causes the structure and function of the myelin sheath to be abnormal. Patients experience weakness and atrophy of the muscles of the lower legs beginning in adolescence; later they experience hand weakness and sensory loss. Interestingly, a different neuropathy distinct from CMT1A called hereditary neuropathy with predisposition to pressure palsy (HNPP) is caused by a deletion of one of the PMP-22 genes. In this case, abnormally low levels of the PMP-22 gene result in episodic, recurrent demyelinating neuropathy. CMT1B is an autosomal dominant disease caused by mutations in the gene that carries the instructions for manufacturing the myelin protein zero (P0), which is another critical component of the myelin sheath. Most of these mutations are point mutations, meaning a mistake occurs in only one letter of the DNA genetic code. To date, scientists have identified more than 30 different point mutations in the P0 gene. As a result of abnormalities in P0, CMT1B produces symptoms similar to those found in CMT1A. The gene defect that causes CMT1C, which also has symptoms similar to those found in CMT1A, has not yet been identified.

In a double-blind, randomized, placebo-controlled pilot study, Sahenk et al. found that subcutaneous administration of the nerve growth factor neurotrophin-3 (NT3; 162660) promoted peripheral nerve regeneration and sensory improvement in 4 patients with CMT1A compared to 4 untreated patients. Sahenk et al., Neurology 2005, 13; 65(5):681-9. Similar results were observed for 2 mouse models of CMT: one with the common PMP22 duplication and one with a PMP22 point mutation. Sahenk et al. concluded that NT3 improved mutant Schwann cell survival and differentiation, resulting in increases in the available Schwann cell pool, which in turn increased the number of myelinated fibers.

There has been considerable recognition that therapeutic compounds which target and activate trk receptors (i.e., trk receptor “agonists”) may be beneficial and desirable. See, for example, Lindsay et al., Exp. Neurol. 1993, 124:103-118; Olson, Neurochem. Int. 1994, 25:1-3. The therapeutic delivery of effective levels of neurotrophins themselves presents considerable challenges because of their large size and short half-life. Moreover, natural neurotrophins may interact with other receptors, such as the p75 receptor in neurons, which is associated with neuronal apoptosis and growth cone collapse. Lee et al., Curr. Opin. Neurobiol. 2001, 11:281-286.

There continues to exist, therefore, a need for compositions that can modulate (i.e., increase or inhibit) neuronal growth and recovery. There also exists a need for processes and methods (including therapeutic methods) that effectively modulate neuronal growth and recovery.

### SUMMARY OF THE INVENTION

This invention relates to agonist antibodies that selectively interact with and activate TrkB function. It is demonstrated for the first time that certain TrkB agonist antibodies are effective in vivo to improve nerve function.

In some embodiments, the invention provides an antibody which specifically binds TrkB and comprises a heavy chain variable region (VH) complementary determining region one (CDR1) having the amino acid sequence GYTFTNYX¹IX² (SEQ ID NO: 159), wherein X¹ is D or V, and X² is I or L; a VH complementary determining region two (CDR2) having the amino acid sequence X¹IX²X³X⁴X⁵X⁶X⁷X⁸X⁹X¹⁰X¹¹X¹²KFKX¹³ (SEQ ID NO: 165), wherein X¹ is Y or H, X² is N, S or A, X³ is P or A, X⁴ is Y or A, X⁵ is N, Q, V or A, X⁶ is G, R, D, A or E, X⁷ is R or G, X⁸ is R, T, K or I, X⁹ is E or K, X¹⁰ is Y, E or A, X¹¹ is N or A, X¹² is E or A, and X¹³ is G or Y; and/or a VH complementary determining region three (CDR3) having the amino acid sequence LLX¹X²X³X⁴X⁵X⁶X⁷X⁸AX⁹X¹⁰X¹¹ (SEQ ID NO: 166), wherein X¹=K, A or R, X² is Y or A, X³ is R or A, X⁴ is R, C, A or P, X⁵ is F, E, A, H, M or L, X⁶ is R, S, A, K, T or Q, X⁷ is Y, E, A or F, X⁸ is Y, E or A, X⁹ is I, E or A, X¹⁰ is D or H, and X¹¹ is Y, E or V. In some embodiments, the VH CDR2 can have the amino acid sequence YINPYNX¹X²X³X⁴YNEKFKG (SEQ ID NO: 160), wherein X₁ is G, R or D, X² is R or G, X³ is R or T, and X⁴ is E or K; and/or the VH CDR3 can have the amino acid sequence LLKYRRFXYYAIDY (SEQ ID NO: 161), wherein X is R or S.

In other embodiments, the invention provides an antibody which specifically binds TrkB and comprises a light chain variable region (VL) CDR1 having the amino acid sequence X¹X²SX³X⁴VX⁵X⁸X⁷X⁸A (SEQ ID NO: 167), wherein X¹ is R or H, X² is A or T, X³ is E, T, S or K, X⁴ is N, P, T, S or A, X⁵ is Y or T, X⁸ is S, R, L, Y, Nor M, X⁷ is N or H, and X⁸ is L, V or T; a VL CDR2 having the amino acid sequence X¹ASNLX²X³ (SEQ ID NO: 168), wherein X¹ is A or I, X² is Q or A, and X³ is S or D; and/or a VL CDR3 having the amino acid sequence QX¹FX²X³X⁴X⁵X⁶X⁷ (SEQ ID NO: 169), wherein X¹ is H, G, N, V, D or Q, X² is W, Y, D, S, K, G or V, X³ is Y, K, V, W, G, Q, A, L, H, M, E, T or D, X⁴ is S, R or L, X⁵ is P, G or W, X⁶ is F, C or W, and X⁷ is T, G, I, K, V, L, A or W. In some embodiments, the VL CDR1 can have the amino acid sequence X¹X²SEX³VYSNX⁴A (SEQ ID NO: 162), wherein X¹ is R or H, X² is A or T, X³ is N or P, and X⁴ is L or V; the VL CDR2 can have the amino acid sequence AASNLX¹X² (SEQ ID NO: 163), wherein X¹ is Q or A, and X² is S or D; and/or the VL CDR3 can have having the amino acid sequence QHFWXSPFT (SEQ ID NO: 164), wherein X is Y or G.

In other embodiments, the invention provides an antibody which specifically binds TrkB and comprises a VL CDR1 having the amino acid sequence X¹X²SX³X⁴VX⁵X⁸X⁷X⁸A (SEQ ID NO: 167), wherein X¹ is R or H, X² is A or T, X³ is E, T, S or K, X⁴ is N, P, T, S or A, X⁵ is Y or T, X⁶ is S, R, L, Y, N or M, X⁷ is N or H, and X⁸ is L, V or T; a VL CDR2 having the amino acid sequence X¹ASNLX²X³ (SEQ ID NO: 168), wherein X¹ is A or I, X² is Q or A, and X³ is S or D; and/or a VL CDR3 having the amino acid sequence QX¹FX²X³X⁴X⁵X⁶X⁷ (SEQ ID NO: 169), wherein X¹ is H, G, N, V, D or Q, X² is W, Y, D, S, K, G or V, X³ is Y, K, V, W, G, Q, A, L, H, M, E, T or D, X⁴ is S, R or L, X⁵ is P, G or W, X⁶ is F, C or W, and X⁷ is T, G, I, K, V, L, A or W, and further comprises a heavy chain variable region VH CDR1 having the amino acid sequence GYTFTNYX¹IX² (SEQ ID NO: 159), wherein X¹ is D or V, and X² is I or L; a VH CDR2 having the amino acid sequence X¹IX²X³X⁴X⁵X⁸X⁷X⁸X⁹X¹⁰X¹¹X¹²KFKX¹³ (SEQ ID NO: 165), wherein X¹ is Y or H, X² is N, S or A, X³ is P or A, X⁴ is Y or A, X⁵ is N, Q, V or A, X⁶ is G, R, D, A or E, X⁷ is R or G, X⁸ is R, T, K or I, X⁹ is E or K, X¹⁰ is Y, E or A, X¹¹ is N or A, X¹² is E or A, and X¹³ is G or Y; and/or a VH CDR3 having the amino acid sequence LLX¹X²X³X⁴X⁵X⁸X⁷X⁸AX⁹X¹⁰X¹¹ (SEQ ID NO: 166), wherein X¹ is K, A or R, X² is Y or A, X³ is R or A, X⁴ is R, C, A or P, X⁵ is F, E, A, H, M or L, X⁶ is R, S, A, K, T or Q, X⁷ is Y, E, A or F, X⁸ is Y, E or A, X⁹ is I, E or A, X¹⁰ is D or H, and X¹¹ is Y, E or V. In some embodiments, the VH CDR2 can have the amino acid sequence YINPYNX¹X²X³X⁴YNEKFKG (SEQ ID NO: 160), wherein X₁ is G, R or D, X² is R or G, X³ is R or T, and X⁴ is E or K; and/or the VH CDR3 can have the amino acid sequence LLKYRRFXYYAIDY (SEQ ID NO: 161), wherein X is R or S. In some embodiments, the VL CDR1 can have the amino acid sequence X¹X²SEX³VYSNX⁴A (SEQ ID NO: 162), wherein X¹ is R or H, X² is A or T, X³ is N or P, and X⁴ is L or V; the VL CDR2 can have the amino acid sequence AASNLX¹X² (SEQ ID NO: 163), wherein X¹ is Q or A, and X² is S or D; the VL CDR1 can have the amino acid sequence QHFWXSPFT (SEQ ID NO: 164), wherein X is Y or G, the VH CDR2 can have the amino acid sequence YINPYNX¹X²X³X⁴YNEKFKG (SEQ ID NO: 160), wherein X¹ is G, R or D, X² is R or G, X³ is R or T, and X⁴ is E or K; and/or the VH CDR3 have the amino acid sequence LLKYRRFXYYAIDY (SEQ ID NO: 161), wherein X is R or S.

In some embodiments, the invention provides an antibody which specifically binds TrkB and comprises a light chain having the amino acid sequence shown in SEQ ID NO: 174; a heavy chain having the amino acid sequence shown in SEQ ID NO: 175, with or without the C-terminal lysine of SEQ ID NO: 175; or both a light chain having the amino acid sequence shown in SEQ ID NO: 174 and a heavy chain having the amino acid sequence shown in SEQ ID NO: 175, with or without the C-terminal lysine of SEQ ID NO: 175.

In some embodiments, the invention provides an antibody which specifically binds TrkB and comprises a VH CDR1 having the amino acid sequence shown in SEQ ID NO: 24, a VH CDR2 having the amino acid sequence shown in SEQ ID NO: 25, and/or a VH CDR3 having the amino acid sequence shown in SEQ ID NO: 3.

The invention is further directed to an antibody comprising a VL CDR1 having the amino acid sequence shown in SEQ ID NO: 19, a CDR2 having the amino acid sequence shown in SEQ ID NO: 5, and/or CDR3 having the amino acid sequence shown in SEQ ID NO: 6. In some embodiments, the antibody comprises a VL CDR1 having the amino acid sequence shown in SEQ ID NO: 19, a CDR2 having the amino acid sequence shown in SEQ ID NO: 5, and/or CDR3 having the amino acid sequence shown in SEQ ID NO: 6, a VH CDR1 having the amino acid sequence shown in SEQ ID NO: 24, a VH CDR2 having the amino acid sequence shown in SEQ ID NO: 25, and a VH CDR3 having the amino acid sequence shown in SEQ ID NO: 3.

The invention is further directed to an antibody comprising a VL CDR1 having the amino acid sequence shown in SEQ ID NO: 4, a CDR2 having the amino acid sequence shown in SEQ ID NO: 5, and/or CDR3 having the amino acid sequence shown in SEQ ID NO: 177. In some embodiments, the antibody comprises a VL CDR1 having the amino acid sequence shown in SEQ ID NO: 4, a CDR2 having the amino acid sequence shown in SEQ ID NO: 5, and/or CDR3 having the amino acid sequence shown in SEQ ID NO: 177, a VH CDR1 having the amino acid sequence shown in SEQ ID NO: 24, a VH CDR2 having the amino acid sequence shown in SEQ ID NO: 25, and a VH CDR3 having the amino acid sequence shown in SEQ ID NO: 3.

In another embodiment, the invention provides an antibody comprising specific VL CDR1, CDR2, and/or CDR3 sequences, and further comprising a VH complementary determining region CDR1 having the amino acid sequence shown in SEQ ID NO: 24, a VH CDR2 having the amino acid sequence shown in SEQ ID NO: 25, and/or VH CDR3 having the amino acid sequence shown in SEQ ID NO: 3.

The invention also provides a humanized antibody comprising a polypeptide having an amino acid sequence selected from the group consisting of DIQMTQSPSSLSASVGDRVTITCX¹X²SX³X⁴VX⁵X⁶X⁷X⁸AWYQQKPGKAPKLLIYX⁹ASN L X¹⁰X¹¹GVPSRFSGSGSGTDX¹²TFTISSLQPEDIATYYCQX¹³FX¹⁴X¹⁵X¹⁶X¹⁷X¹⁸X¹⁹FGQG TKLEIK (SEQ ID NO: 20), wherein X¹ is R or H, X² is A or T, X³ is E, T, S or K, X⁴ is N, P, T, S or A, X⁵ is Y or T, X⁶ is S, R, L, Y, N or M, X⁷ is N or H, X⁸ is L, V or T, X⁹ is A or I, X¹⁰ is Q or A, X¹¹ is S or D, X¹² is F or Y, X¹³ is H, G, N, V, D or Q, X¹⁴ is W, Y, D, S, K, G or V, X¹⁵ is Y, K, V, W, G, Q, A, L, H, M, E, T or D, X¹⁶ is S, R or L, X¹⁷ is P, G or W, X¹⁸ is F, C or W, and X¹⁹ is T, G, I, K, V, L, A or W; QVQLVQSGAEVKKPGASVKVSCKASGYTFTNYX¹IX²WVRQAPGQGLEWMGX³IX⁴X⁵X⁶X⁷X⁸X⁹X¹⁰X¹¹X¹²X¹³X¹⁴KFKX¹⁵RVTMTRDTSTSTVYMELSSLRSEDTAVYYCARLLX¹⁶X¹⁷X¹⁸X¹⁹X²⁰X²¹X²²X²³AX²⁴X²⁵X²⁶WGQGTTVTVSS (SEQ ID NO: 21), wherein X¹ is D or V, X² is I or L, X³ is Y or H, X⁴ is N, S or A, X⁵ is P or A, X⁶ is Y or A, X⁷ is N, Q, V or A, X⁸ is G, R, D, A or E, X⁹ is R or G, X¹⁰ is R, T, K or I, X¹¹ is E or K, X¹² is Y, E or A, X¹³ is N or A, X¹⁴ is E or A, X¹⁵ is G or Y, X¹⁶ is K, A or R, X¹⁷ is Y or A, X¹⁸ is R or A, X¹⁹ is R, C, A or P, X²⁰ is F, E, A, H, M or L, X²¹ is R, S, A, K, T or Q, X²² is Y, E, A or F, X²³ is Y, E, or A, X²⁴ is I, E or A, X²⁵ is D or H, and X²⁶ is Y, E or V; and both SEQ ID NO: 20 and SEQ ID NO: 21.

The invention also provides a humanized antibody comprising a polypeptide having an amino acid sequence selected from the group consisting of DIQMTQSPSSLSASVGDRVTITCX¹X²SEX³VYSNX⁴AWYQQKPGKAPKLLIYAASNLX⁵X⁶GVPSRFSGSGSGTDX⁷TFTISSLQPEDIATYYCQHFWX⁸SPFTFGQGTKLEIK (SEQ ID NO: 170), wherein X¹ is R or H, X² is A or T, X³ is N or P, X⁴ is L or V, X⁵ is Q or A, X⁶ is S or D; X⁷ is F or Y, and X⁸ is Y or G; QVQLVQSGAEVKKPGASVKVSCKASGYTFTNYX¹IX²WVRQAPGQGLEWMGYINPYN X³X⁴X⁵X⁶YNEKFKGRVTMTRDTSTSTVYMELSSLRSEDTAVYYCARLLKYRRFX⁷YYAID YWGQGTTVTVSS (SEQ ID NO: 171), wherein X¹ is D or V, X² is I or L, X³ is G, R or D, X⁴ is R or G, X⁵ is R or T, X⁶ is E or K, and X⁷ is R or S; and both SEQ ID NO: 170 and SEQ ID NO: 171.

The invention also provides a humanized antibody comprising a polypeptide having an amino acid sequence selected from the group consisting of SEQ ID NO: 173, SEQ ID NO: 12, both SEQ ID NO: 173 and SEQ ID NO: 12, and a variant thereof having one or more conservative amino acid substitutions in said sequences, wherein the variant retains essentially the same binding specificity as the antibody defined by said sequence(s). The invention also provides a humanized antibody comprising a polypeptide having an amino acid sequence selected from the group consisting of SEQ ID NO: 176, SEQ ID NO: 12, both SEQ ID NO: 176 and SEQ ID NO: 12, and a variant thereof having one or more conservative amino acid substitutions in said sequences, wherein the variant retains essentially the same binding specificity as the antibody defined by said sequence(s).

In some embodiments the antibody is capable of promoting neuron survival. In some embodiments the antibody, when administered to a subject, is capable of improving nerve function in said subject.

In some embodiments the antibody, when administered to a subject, is capable of increasing compound muscle action potential (CMAP) area in said subject.

In some embodiments, the antibody can bind to human TrkB. In some embodiments, the antibody blocks BDNF binding to TrkB.

In some embodiments, the antibody can have an immunologically inert constant region. In some embodiments, the antibody can have an isotype that is selected from the group consisting of IgG₂, IgG₄, IgG_{2Δa}, IgG_{4Δb}, IgG_{4Δc}, IgG₄ S228P, Ig_{4Δb} S228P and IgG_{4Δc} S228P.

In other embodiments, the invention provides a monoclonal antibody comprising a partial heavy chain encoded by the polynucleotide having ATCC Accession No. PTA-10276, and a partial light chain encoded by the polynucleotide having ATCC Accession No. PTA-10277.

In some embodiments, the invention provides an humanized agonist anti-TrkB antibody which comprises an antibody which binds to TrkB with an equilibrium dissociation constant of less than about 20 nM, and wherein the humanized antibody overlaps the same epitope of TrkB that is recognized by monoclonal antibody 38B8, which is produced by a hybridoma cell line deposited with the American Type Culture Collection and assigned accession number PTA-8766. In some embodiments, the antibody binds to TrkB with an equilibrium dissociation constant of less than about 1 nM, less than about 50 pM or about 20 pM.

In some embodiments, the invention provides a humanized antibody that comprises an antigen binding region that competes with a monoclonal antibody 38B8, which is produced by a hybridoma cell line deposited with the American Type Culture Collection and assigned accession number PTA-8766, for binding to TrkB.

The invention also provides a pharmaceutical composition comprising a humanized TrkB antibody as described herein. The invention also provides a cell line that recombinantly produces a TrkB antibody as described herein. The invention also provides a nucleic acid encoding the antibody as described herein.

In some embodiments, the invention provides a method for improving nerve function in a subject in need thereof, comprising administering to the subject a therapeutically effective amount of an agonist anti-TrkB antibody as disclosed herein. In some embodiments, the agonist anti-TrkB monoclonal antibody can have an equilibrium dissociation constant of less than about 20 nM. In some embodiments, the agonist anti-TrkB monoclonal antibody shows no significant cross-reactivity with TrkA or TrkC.

The invention further provides methods for preventing and/or treating a peripheral neuropathy in a patient, comprising administering to the patient a therapeutically effective amount of the antibody prepared according to the invention. In some embodiments, the invention provides an agonist anti-TrkB antibody for use in a method for preventing and/or treating a peripheral neuropathy in a subject. In some embodiments, the peripheral neuropathy can be CMT disease.

Further provided are methods for preventing and/or treating CMT disease in a patient, comprising administering to the patient a therapeutically effective amount of a TrkB agonist, thereby ameliorating one or more symptoms of CMT disease. In some embodiments, the invention provides a TrkB agonist for use in a method for preventing and/or treating CMT disease. In some embodiments, the TrkB agonist can be an agonist anti-TrkB antibody. In some embodiments, the CMT disease is selected from the group consisting of CMT1, CMT2, CMT3, CMT4 and CMTX. In some embodiments, CMT1 is selected from the group consisting of CMT1A, CMT1B and CMT1C. In a preferred embodiment, the patient is a human patient.

In some embodiments, the method can further comprise improving muscle strength in a subject following administration of the TrkB agonist. In some embodiments, the method can further comprise improving grip strength in a subject.

In some embodiments, the TrkB agonist can be administered parenterally.

Further provided are methods for increasing CMAP area in a subject in need thereof. The methods comprise administering to the subject a therapeutically effective amount of an agonist anti-TrkB monoclonal antibody. In a preferred embodiment, the subject is a human subject. In some embodiments, the subject can be suffering from a peripheral neuropathy. In some embodiments, the peripheral neuropathy can be CMT disease.

In some embodiments, the agonist anti-TrkB antibody administered can be a humanized antibody.

In some embodiments, the method can further comprise administering to the subject a therapeutically effective amount of a TrkC agonist antibody.

The invention further provides methods for increasing body weight and/or food intake by peripheral administration of an agonist anti-TrkB antibody, including an anti-TrkB selective agonist antibody. In some embodiments, the invention provides an agonist anti-TrkB antibody for use in methods for increasing body weight and/or food intake in a primate. These methods can be used for treating or preventing unwanted weight loss (such as with cachexia or with aging), eating disorders (such as anorexia nervosa), and opioid-induced emesis.

In other embodiments, the invention provides a method for increasing body weight in a primate, the method comprising peripherally administering to the primate an effective amount of an agonist anti-TrkB antibody.

In other embodiments, the invention provides methods for increasing food intake in a primate comprising peripherally administering to the primate an effective amount of an agonist anti-TrkB antibody.

In other embodiments, the invention provides a method for treating or preventing cachexia in a primate, the method comprising peripherally administering to the primate an effective amount of an agonist anti-TrkB antibody, thereby ameliorating one or more symptoms of cachexia. Preferably, the primate is suffering from or is at risk for cachexia. In some embodiments, the cachexia is related to and/or caused by cancer or cancer treatment. In some embodiments, the invention provides an agonist anti-TrkB antibody for use in a method for treating cachexia.

In other embodiments, the invention provides a method for ameliorating, reducing incidence of, or delaying the development or progression of cachexia in a primate, the method comprising peripherally administering to the primate an effective amount of an agonist anti-TrkB antibody.

In other embodiments, the invention provides a method for treating unwanted weight loss in a primate, the method comprising peripherally administering to the primate an effective amount of an agonist anti-TrkB antibody. Preferably, the primate is suffering from or is at risk for unwanted weight loss. In some embodiments, the unwanted weight loss is related to and/or caused by cancer or cancer treatment.

In other embodiments, the invention provides methods for ameliorating, reducing incidence of, or delaying the development or progression of unwanted weight loss in a primate comprising peripherally administering to the primate an effective amount of an agonist anti-TrkB antibody.

In other embodiments, the invention provides a method for treating or preventing anorexia nervosa in a primate, the method comprising peripherally administering to the primate an effective amount of an agonist anti-TrkB antibody. Preferably, the primate is suffering from or is at risk for anorexia nervosa.

In other embodiments, the invention provides a method for ameliorating, reducing incidence of, or delaying the development or progression of anorexia nervosa in a primate, the method comprising peripherally administering to the primate an effective amount of an agonist anti-TrkB antibody.

In other embodiments, the invention provides a method for treating or preventing opioid-induced emesis in an individual, the method comprising peripherally administering to the individual an effective amount of an agonist anti-TrkB antibody. Preferably, the primate is suffering from or is at risk for opioid-induced emesis.

In other embodiments, the invention provides a method for ameliorating, reducing incidence of, or delaying the development or progression of opioid-induced emesis in an individual, the method comprising peripherally administering to the individual an effective amount of an agonist anti-TrkB antibody.

In other embodiments, the agonist anti-TrkB antibody can be administered peripherally. For example, the agonist anti-TrkB antibody may be administered by one of the following means: intravenously, intraperitoneally, intramuscularly, subcutaneously, parenterally, via inhalation, intraarterially, intracardially, intraventricularly, and transdermally.

The invention further provides methods for treating and/or preventing glaucoma and/or ischemic retinopathy in an individual by administration of an agonist anti-TrkB antibody. In some embodiments, the agonist anti-TrkB antibody can be administered intravitreally. In some embodiments, the invention provides an agonist anti-TrkB antibody for use in methods for treating and/or preventing glaucoma in an individual. Preferably, the individual is suffering from glaucoma. In some embodiments, the glaucoma is primary angle closure glaucoma. In some embodiments, the individual is suffering from ischemic retinopathy.

In some embodiments, the individual is a primate. In some embodiments, the primate is a human.

### BRIEF DESCRIPTION OF THE FIGURES/DRAWINGS

FIG. 1 depicts graphs showing the relative amount of neuron survival in the presence of increasing amounts of several agonist anti-TrkB humanized antibodies using an in vitro assay. Top graph: agonist anti-TrkB humanized antibodies C1, C2, 4A6 and 4B12. Bottom graph: various isotype forms of agonist anti-TrkB humanized antibody RN1026A (1026A). Mouse monoclonal agonist anti-TrkB antibody 38B8 and/or BDNF are used positive controls. The y-axis indicates the number of neurons, and x-axis indicates the concentration of antibody (or BDNF) in pM. The experimental procedures and results are described in Example 2.

FIG. 2 depicts the results of treatment of young trembler mice with agonist anti-TrkB antibody (TrkB), agonist anti-TrkC antibody (TrkC), or both TrkB and TrkC. The y-axis indicates grip strength force in grams, and x-axis indicates length of treatment in weeks.

FIG. 3 depicts the results of treatment of young trembler mice with agonist anti-TrkB antibody (TrkB-AAb), agonist anti-TrkC antibody (TrkC-AAb), or both antibodies (TrkB+TrkC). The y-axis indicates the CMAP Area (mV/ms), and x-axis indicates ipsilateral grip strength in grams.

FIG. 4 depicts the results of treatment of young trembler mice with agonist anti-TrkB antibody (TrkB-AAb), agonist anti-TrkC antibody (TrkC-AAb), or both antibodies (TrkB+TrkC). The y-axis indicates the conduction velocity (m/s), and x-axis indicates ipsilateral grip strength in grams.

FIG. 5 depicts a graph showing the effect on body weight in high fat diet-induced obesity (DIO) mice after intraperitoneal injections of agonist anti-TrkB agonist antibody. Antibody or vehicle control was injected on day 0. Body weight was measured on days 1, 2, 3, 6, 7 and 13 after dosing. “***” indicates P<0.001 as compared to vehicle; “**” indicates P<0.01 as compared to vehicle.

FIG. 6 depicts a graph showing the effect on body weight in DIO mice after intraperitoneal injections of agonist anti-TrkB agonist antibody at various dosages. Antibody or vehicle was injected on day 0. Body weight was measured on days 1, 2, 3, 4 and 7 after dosing. “***” indicates P<0.001 as compared to vehicle; “**” indicates P<0.01 as compared to vehicle.

FIG. 7 depicts a graph showing antibody concentrations in serum collected from DIO mice treated with agonist anti-TrkB antibody. DIO mice were bled for serum collection at various time points after intraperitoneal injection of anti-trkB agonist antibody.

FIGS. 8A-D depict graphs showing the effect on retinal ganglion cell (RGC) survival after transient glaucomatous injury and intravitreal injection of agonist anti-TrkB antibody. (A) Average density of RGCs per mm² in the entire retina. (B) Average density of RGCs per mm² in the nasal half of retina. (C) Fraction of surviving RGCs (relative to non-glaucomatous contralateral eye) in the entire retina. (D) Fraction of surviving RGCs in the nasal half of retina. “*” indicates P<0.05.

### DETAILED DESCRIPTION OF THE INVENTION

The present invention relates to antibodies that promote the function of TrkB. More specifically, the invention relates to methods of making agonist anti-TrkB antibodies, including humanized agonist anti-TrkB antibodies, compositions comprising these antibodies, and methods of using these antibodies as a medicament. The agonist anti-TrkB antibodies can be used to improve neuron function, and can be used in the prevention and/or treatment of peripheral neuropathies, including, for example, Charcot-Marie-Tooth (CMT) Disease.

### General Techniques

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995).

### DEFINITIONS

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also fragments thereof (such as Fab, Fab′, F(ab′)₂, Fv), single chain (ScFv) and domain antibodies), and fusion proteins comprising an antibody portion (such as domain antibodies), and any other modified configuration of the immunoglobulin molecule that comprises an antigen recognition site. An antibody includes an antibody of any class, such as IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

As used herein, “monoclonal antibody” refers to an antibody obtained from a population of substantially homogeneous antibodies, i.e., the individual antibodies comprising the population are identical except for possible naturally-occurring mutations that may be present in minor amounts. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler and Milstein, 1975, Nature 256:495, or may be made by recombinant DNA methods such as described in U.S. Pat. No. 4,816,567. The monoclonal antibodies may also be isolated from phage libraries generated using the techniques described in McCafferty et al., 1990, Nature 348:552-554, for example.

As used herein, “humanized” antibody refers to forms of non-human (e.g. murine) antibodies that are chimeric immunoglobulins, immunoglobulin chains, or fragments thereof (such as Fv, Fab, Fab′, F(ab′)₂ or other antigen-binding subsequences of antibodies) that contain minimal sequence derived from non-human immunoglobulin. Preferably, humanized antibodies are human immunoglobulins (recipient antibody) in which residues from a complementary determining region (CDR) of the recipient are replaced by residues from a CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Preferred are antibodies having Fc regions modified as described in WO 99/58572. Other forms of humanized antibodies have one or more CDRs (CDR L1, CDR L2, CDR L3, CDR H1, CDR H2, or CDR H3) which are altered with respect to the original antibody, which are also termed one or more CDRs “derived from” one or more CDRs from the original antibody.

As used herein, “human antibody” means an antibody having an amino acid sequence corresponding to that of an antibody produced by a human and/or which has been made using any of the techniques for making human antibodies known to those skilled in the art or disclosed herein. This definition of a human antibody includes antibodies comprising at least one human heavy chain polypeptide or at least one human light chain polypeptide. One such example is an antibody comprising murine light chain and human heavy chain polypeptides. Human antibodies can be produced using various techniques known in the art. In some embodiments, the human antibody is selected from a phage library, where that phage library expresses human antibodies (Vaughan et al., 1996, Nature Biotechnology, 14:309-314; Sheets et al., 1998, Proc. Natl. Acad. Sci. (USA) 95:6157-6162; Hoogenboom and Winter, 1991, J. Mol. Biol., 227:381; Marks et al., 1991, J. Mol. Biol., 222:581). Human antibodies can also be made by immunization of animals into which human immunoglobulin loci have been transgenically introduced in place of the endogenous loci, e.g., mice in which the endogenous immunoglobulin genes have been partially or completely inactivated. This approach is described in U.S. Pat. Nos. 5,545,807; 5,545,806; 5,569,825; 5,625,126; 5,633,425; and 5,661,016. Alternatively, the human antibody may be prepared by immortalizing human B lymphocytes that produce an antibody directed against a target antigen (such B lymphocytes may be recovered from an individual or may have been immunized in vitro). See, e.g., Cole et al. Monoclonal Antibodies and Cancer Therapy, Alan R. Liss, p. 77, 1985; Boerner et al., 1991, J. Immunol., 147 (1):86-95; and U.S. Pat. No. 5,750,373.

As used herein, the term “TrkB” refers to any form of TrkB and variants thereof that retain at least part of the activity of TrkB. Unless indicated differently, such as by specific reference to human TrkB, TrkB includes all mammalian species of native sequence TrkB, e.g., human, canine, feline, equine, and bovine.

As used herein, a “TrkB agonist” refers to an antibody, peptide, or aptamer that is able to bind to TrkB and activate TrkB biological activity and/or downstream pathway(s) mediated by the TrkB signaling function. For example, the agonist may bind to the extracellular domain of a TrkB receptor and thereby cause dimerization of the receptor, resulting in activation of the intracellular catalytic kinase domain. Consequently, this may result in stimulation of growth and/or differentiation of cells expressing the receptor in vitro and/or in vivo. In some embodiments, a TrkB agonist binds to TrkB and activates a TrkB biological activity. TrkB agonists include naturally-occurring agonist polypeptides, fragments, variants, and derivatives, thereof, including but not limited to the known TrkB agonists NT4 and BDNF. TrkB agonists include agonist antibodies, fragments, variants, and derivatives, thereof. Preferred properties of TrkB agonists are described herein. TrkB agonists of the invention may increase activation of TrkB by at least 5%, at least 10%, at least 20%, at least 30%, at least 50%, at least 100%, at least 200%, or more.

As used herein, “naturally-occurring TrkB agonists” are molecules that exist in nature and function as activators of TrkB receptors. The known naturally-occurring agonists of TrkB receptors are the neurotrophins NT4 and BDNF. Naturally-occurring TrkB agonists include naturally-occurring variant molecules, such as a neurotrophins polypeptide expressed in an animal with a mutated TrkB allele.

“Biological activity”, when used in conjunction with the TrkB agonist of the present invention, generally refers to having the ability to bind and activate the TrkB receptor and/or a downstream pathway mediated by the TrkB signaling function. As used herein, “biological activity” encompasses one or more effector functions in common with those induced by action of NT-3, NT-4/5 and/or BDNF, the native ligand of TrkB, on a TrkB-expressing cell. Without limitation, biological activities include any one or more of the following: ability to bind and activate TrkB; ability to promote TrkB receptor dimerization; the ability to promote the development, survival, function, maintenance and/or regeneration of cells (including damaged cells), in particular neurons in vitro or in vivo, including peripheral (sympathetic, sensory, motor, and enteric) neurons, and central (brain and spinal cord) neurons, and non-neuronal cells, e.g. peripheral blood leukocytes, endothelial cells and vascular smooth muscle cells. A particular preferred biological activity is the ability to improve nerve function in a mammal when administered peripherally, to treat (including prevention of) one or more symptoms of peripheral neuropathy in a mammal.

A “TrkB agonist antibody” (interchangeably termed “agonist TrkB antibody,” “agonist anti-TrkB antibody” or “anti-TrkB agonist antibody”) refers to an antibody that is able to bind to and activate a TrkB receptor and/or downstream pathway(s) mediated by the TrkB signaling function. For example, the agonist antibody may bind to the extracellular domain of a TrkB receptor and thereby cause dimerization of the receptor, resulting in activation of the intracellular catalytic kinase domain. Consequently, this may result in stimulation of growth and/or differentiation of cells expressing the receptor in vitro and/or in vivo. In some embodiments, an agonist anti-TrkB antibody binds to TrkB and activates a TrkB biological activity. A TrkB agonist antibody encompasses antibodies that activate, promote or induce (to any degree including significantly) TrkB biological activity, including downstream pathways mediated by TrkB signaling. For purposes of the present invention, it will be explicitly understood that the term “TrkB agonist antibody” encompasses all the previously identified terms, titles, and functional states and characteristics whereby the TrkB itself, a TrkB biological activity, or the consequences of the biological activity, are substantially induced, increased or activated in any meaningful degree. Examples of TrkB agonist antibodies are provided herein.

As used herein a “full agonist” is an agonist which, at an effective concentration, essentially completely induces a measurable effect of TrkB. For example, the measurable effect of TrkB may be neuronal survival or increased muscle strength. By a partial agonist is meant an agonist that is capable of partially inducing a measurable effect, but that, even at a highest concentration is not a full agonist. By essentially completely is meant at least about 80%, preferably, at least about 90%, more preferably, at least about 95%, and most preferably, at least about 98% of the measurable effect is induced. The relevant “measurable effects” are described herein.

The terms “polypeptide”, “oligopeptide”, “peptide” and “protein” are used interchangeably herein to refer to chains of amino acids of any length, preferably, relatively short (e.g., 10-100 amino acids). The chain may be linear or branched, it may comprise modified amino acids, and/or may be interrupted by non-amino acids. The terms also encompass an amino acid chain that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that the polypeptides can occur as single chains or associated chains.

As known in the art, “polynucleotide,” or “nucleic acid,” as used interchangeably herein, refer to chains of nucleotides of any length, and include DNA and RNA. The nucleotides can be deoxyribonucleotides, ribonucleotides, modified nucleotides or bases, and/or their analogs, or any substrate that can be incorporated into a chain by DNA or RNA polymerase. A polynucleotide may comprise modified nucleotides, such as methylated nucleotides and their analogs. If present, modification to the nucleotide structure may be imparted before or after assembly of the chain. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, alpha- or beta-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S(“thioate”), P(S)S (“dithioate”), (O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

A “variable region” of an antibody refers to the variable region of the antibody light chain or the variable region of the antibody heavy chain, either alone or in combination. As known in the art, the variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen-binding site of antibodies. There are at least two techniques for determining CDRs: (1) an approach based on cross-species sequence variability (i.e., Kabat et al. Sequences of Proteins of Immunological Interest, (5th ed., 1991, National Institutes of Health, Bethesda Md.)); and (2) an approach based on crystallographic studies of antigen-antibody complexes (Al-lazikani et al (1997) J. Molec. Biol. 273:927-948). As used herein, a CDR may refer to CDRs defined by either approach or by a combination of both approaches.

As known in the art a “constant region” of an antibody refers to the constant region of the antibody light chain or the constant region of the antibody heavy chain, either alone or in combination.

As used herein, an antibody “interacts with” TrkB when the equilibrium dissociation constant is equal to or less than 20 nM, preferably less than about 6 nM, more preferably less than about 1 nM, most preferably less than about 0.2 nM, as measured by the methods disclosed herein.

An epitope that “preferentially binds” or “specifically binds” (used interchangeably herein) to an antibody or a polypeptide is a term well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. For example, an antibody that specifically or preferentially binds to a TrkB epitope is an antibody that binds this epitope with greater affinity, avidity, more readily, and/or with greater duration than it binds to other TrkB epitopes or non-TrkB epitopes. It is also understood by reading this definition that, for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding.

As used herein, “substantially pure” refers to material which is at least 50% pure (i.e., free from contaminants), more preferably, at least 90% pure, more preferably, at least 95% pure, yet more preferably, at least 98% pure, and most preferably, at least 99% pure.

A “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of this invention.

As known in the art, the term “Fc region” is used to define a C-terminal region of an immunoglobulin heavy chain. The “Fc region” may be a native sequence Fc region or a variant Fc region. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The numbering of the residues in the Fc region is that of the EU index as in Kabat. Kabat et al., Sequences of Proteins of Immunological Interest, 5th Ed. Public Health Service, National Institutes of Health, Bethesda, Md., 1991. The Fc region of an immunoglobulin generally comprises two constant domains, CH2 and CH3.

As used in the art, “Fc receptor” and “FcR” describe a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. FcRs are reviewed in Ravetch and Kinet, 1991, Ann. Rev. Immunol., 9:457-92; Capel et al., 1994, Immunomethods, 4:25-34; and de Haas et al., 1995, J. Lab. Clin. Med., 126:330-41. “FcR” also includes the neonatal receptor, FcRn, which is responsible for the transfer of maternal IgGs to the fetus (Guyer et al., 1976, J. Immunol., 117:587; and Kim et al., 1994, J. Immunol., 24:249).

The term “compete”, as used herein with regard to an antibody, means that a first antibody, or an antigen-binding portion thereof, binds to an epitope in a manner sufficiently similar to the binding of a second antibody, or an antigen-binding portion thereof, such that the result of binding of the first antibody with its cognate epitope is detectably decreased in the presence of the second antibody compared to the binding of the first antibody in the absence of the second antibody. The alternative, where the binding of the second antibody to its epitope is also detectably decreased in the presence of the first antibody, can, but need not be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope or ligand, whether to the same, greater, or lesser extent, the antibodies are said to “cross-compete” with each other for binding of their respective epitope(s). Both competing and cross-competing antibodies are encompassed by the present invention. Regardless of the mechanism by which such competition or cross-competition occurs (e.g., steric hindrance, conformational change, or binding to a common epitope, or portion thereof), the skilled artisan would appreciate, based upon the teachings provided herein, that such competing and/or cross-competing antibodies are encompassed and can be useful for the methods disclosed herein.

A “functional Fc region” possesses at least one effector function of a native sequence Fc region. Exemplary “effector functions” include C1q binding; complement dependent cytotoxicity; Fc receptor binding; antibody-dependent cell-mediated cytotoxicity; phagocytosis; down-regulation of cell surface receptors (e.g. B cell receptor), etc. Such effector functions generally require the Fc region to be combined with a binding domain (e.g. an antibody variable domain) and can be assessed using various assays known in the art for evaluating such antibody effector functions.

A “native sequence Fc region” comprises an amino acid sequence identical to the amino acid sequence of an Fc region found in nature. A “variant Fc region” comprises an amino acid sequence which differs from that of a native sequence Fc region by virtue of at least one amino acid modification, yet retains at least one effector function of the native sequence Fc region. Preferably, the variant Fc region has at least one amino acid substitution compared to a native sequence Fc region or to the Fc region of a parent polypeptide, e.g. from about one to about ten amino acid substitutions, and preferably, from about one to about five amino acid substitutions in a native sequence Fc region or in the Fc region of the parent polypeptide. The variant Fc region herein will preferably possess at least about 80% sequence identity with a native sequence Fc region and/or with an Fc region of a parent polypeptide, and most preferably, at least about 90% sequence identity therewith, more preferably, at least about 95%, at least about 96%, at least about 97%, at least about 98%, at least about 99% sequence identity therewith.

As used herein, “treatment” is an approach for obtaining beneficial or desired clinical results. For purposes of this invention, beneficial or desired clinical results include, but are not limited to, one or more of the following: increasing muscle strength, increasing nerve function, improving mobility, and reducing incidence or amelioration of tremor and/or ataxia resulting from peripheral neuropathies, including CMT disease.

“Reducing incidence” means any of reducing severity (which can include reducing need for and/or amount of (e.g., exposure to) other drugs and/or therapies generally used for this condition. As is understood by those skilled in the art, individuals may vary in terms of their response to treatment, and, as such, for example, a “method of reducing incidence” reflects administering the TrkB agonist antibody based on a reasonable expectation that such administration may likely cause such a reduction in incidence in that particular individual.

“Ameliorating” means a lessening or improvement of one or more symptoms as compared to not administering a TrkB agonist antibody. “Ameliorating” also includes shortening or reduction in duration of a symptom.

As used herein, “improving nerve function” refers to increasing the compound muscle action potential (CMAP) amplitude and/or conduction velocity (CV) in a nerve. In some embodiments, improving nerve function also refers to increasing the number and/or size of axons present in a nerve, increasing nerve regeneration and/or improving the morphology of axons in a nerve. An increase in CMAP area can be used as an accurate indicator of nerve function. See, Muscle Nerve, 2008, 38:1254-1265. In some embodiments, CMAP area can be used as a parameter of nerve regeneration and re-innervation. The level of improvement in nerve function may vary. For example, improved nerve function may be measured by about 10%, about 20%, about 30%, about 40%, about 50%, about 60%, about 70%, about 80%, about 90%, or about 95%, in comparative or actual increase in, for example, CMAP area.

As used herein, an “effective dosage” or “effective amount” of drug, compound, or pharmaceutical composition is an amount sufficient to effect any one or more beneficial or desired results. For prophylactic use, beneficial or desired results include eliminating or reducing the risk, lessening the severity, or delaying the outset of the disease, including biochemical, histological and/or behavioral symptoms of the disease, its complications and intermediate pathological phenotypes presenting during development of the disease. For therapeutic use, beneficial or desired results include clinical results such as, for example, reducing one or more symptoms of CMT disease, decreasing the dose of other medications required to treat the disease, enhancing the effect of another medication, and/or delaying the progression of the disease of patients. An effective dosage can be administered in one or more administrations. For purposes of this invention, an effective dosage of drug, compound, or pharmaceutical composition is an amount sufficient to accomplish prophylactic or therapeutic treatment either directly or indirectly. As is understood in the clinical context, an effective dosage of a drug, compound, or pharmaceutical composition may or may not be achieved in conjunction with another drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved.

An “individual” or a “subject” is a mammal, more preferably, a human. Mammals also include, but are not limited to, farm animals, sport animals, pets, primates, horses, dogs, cats, mice and rats.

As used herein, “vector” means a construct, which is capable of delivering, and, preferably, expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

As used herein, “expression control sequence” means a nucleic acid sequence that directs transcription of a nucleic acid. An expression control sequence can be a promoter, such as a constitutive or an inducible promoter, or an enhancer. The expression control sequence is operably linked to the nucleic acid sequence to be transcribed.

As used herein, “pharmaceutically acceptable carrier” or “pharmaceutical acceptable excipient” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline (PBS) or normal (0.9%) saline. Compositions comprising such carriers are formulated by well known conventional methods (see, for example, Remington's Pharmaceutical Sciences, 18th edition, A. Gennaro, ed., Mack Publishing Co., Easton, Pa., 1990; and Remington, The Science and Practice of Pharmacy 20th Ed. Mack Publishing, 2000).

As used herein, “peripheral administration” or “administered peripherally” refers to introducing an agent into a subject outside of the central nervous system (CNS) or blood brain barrier (BBB). Peripheral administration encompasses any route of administration other than direct administration to the spine or brain. Peripheral administration can be local or systemic.

The term “kₒₙ”, as used herein, refers to the rate constant for association of an antibody to an antigen. Specifically, the rate constants (kₒₙ and k_{off}) and equilibrium dissociation constants are measured using Fab antibody fragments (i.e. univalent) and TrkB.

The term “k_{off}”, as used herein, refers to the rate constant for dissociation of an antibody from the antibody/antigen complex.

The term “K_{D}”, as used herein, refers to the equilibrium dissociation constant of an antibody-antigen interaction.

### A. Methods for Preventing or Treating a Peripheral Neuropathy

In one aspect, the invention provides a method for treating or preventing a peripheral neuropathy, including Charcot-Marie-Tooth disease, in an individual comprising administering to the individual an effective amount of a TrkB agonist antibody.

Advantageously, therapeutic administration of the TrkB agonist antibody results in increased compound muscle action potential (CMAP) area. Changes in CMAP area reflects the function of axons of alpha motor neurons and can readily be measured by electrophysiological methods known in the art. Nerve function and grip strength positively correlates with CMAP area and conduction velocity (CV) upon treatment with TrkB agonist antibody. See, e.g., Example 4 below. CVs, CMAP amplitudes, CMAP duration, and the shape of the CMAP may be used as parameters of nerve function. Any suitable methods known in the art, such as electrophysiological methods, may be used to measure CMAP area, conduction velocities, CMAP amplitudes and CMAP duration. CMAP area, conduction velocity and CMAP amplitude can be measured in any suitable area of a subject, including, for example without limitation, the sciatic nerve.

Preferably, CMAP area is at least about 10% greater than before administration. More preferably, CMAP area is at least about 20% greater than before administration of the antibody. Yet more preferably, CMAP area is at least about 30% greater than before administration of the antibody. Advantageously, CMAP area is at least about 40% greater than before administration of the antibody. More advantageously, CMAP area is at least about 50% greater than before administration of the antibody. Very preferably, CMAP area is at least about 60% greater than before administration of the antibody. Most preferably, CMAP area is at least about 70% greater than before administration of the antibody. Similarly, conduction velocity is at least about 10% greater than before administration. More preferably, conduction velocity is at least about 20% greater than before administration of the antibody. Yet more preferably, conduction velocity is at least about 30% greater than before administration of the antibody. Advantageously, conduction velocity is at least about 40% greater than before administration of the antibody. More advantageously, conduction velocity is at least about 50% greater than before administration of the antibody. Very preferably, conduction velocity is at least about 60% greater than before administration of the antibody. Most preferably, conduction velocity is at least about 70% greater than before administration of the antibody. Similarly, CMAP amplitude is at least about 10% greater than before administration. More preferably, CMAP amplitude is at least about 20% greater than before administration of the antibody. Yet more preferably, CMAP amplitude is at least about 30% greater than before administration of the antibody. Advantageously, CMAP amplitude is at least about 40% greater than before administration of the antibody. More advantageously, CMAP amplitude is at least about 50% greater than before administration of the antibody. Very preferably, CMAP amplitude is at least about 60% greater than before administration of the antibody. Most preferably, CMAP amplitude is at least about 70% greater than before administration of the antibody.

In some embodiments, therapeutic administration of the TrkB agonist antibody can result in increased muscle and/or grip strength. In some embodiments, therapeutic administration of the TrkB agonist antibody can result in peripheral nerve regeneration. In some embodiments, therapeutic administration of the TrkB agonist antibody can result in increased myelin sheath thickness. In some embodiments, therapeutic administration of the TrkB agonist antibody can result in improved axon morphology.

With respect to all methods described herein, reference to TrkB agonist antibodies also includes compositions comprising one or more additional agents. These compositions may further comprise suitable excipients, such as pharmaceutically acceptable excipients including buffers, which are well known in the art. The present invention can be used alone or in combination with other conventional methods of treatment.

The TrkB agonist antibody can be administered to an individual via any suitable route. It should be apparent to a person skilled in the art that the examples described herein are not intended to be limiting but to be illustrative of the techniques available. Accordingly, in some embodiments, the TrkB agonist antibody is administered to an individual in accord with known methods, such as intravenous administration, e.g., as a bolus or by continuous infusion over a period of time, by intramuscular, intraperitoneal, intracerebrospinal, transdermal, subcutaneous, intra-articular, sublingually, intrasynovial, via insufflation, intrathecal, oral, inhalation or topical routes. Administration can be systemic, e.g., intravenous administration, or localized. Commercially available nebulizers for liquid formulations, including jet nebulizers and ultrasonic nebulizers are useful for administration. Liquid formulations can be directly nebulized and lyophilized powder can be nebulized after reconstitution. Alternatively, TrkB agonist antibody can be aerosolized using a fluorocarbon formulation and a metered dose inhaler, or inhaled as a lyophilized and milled powder.

In some embodiments, a TrkB agonist antibody is administered via site-specific or targeted local delivery techniques. Examples of site-specific or targeted local delivery techniques include various implantable depot sources of the TrkB agonist antibody or local delivery catheters, such as infusion catheters, indwelling catheters, or needle catheters, synthetic grafts, adventitial wraps, shunts and stents or other implantable devices, site specific carriers, direct injection, or direct application. See, e.g., PCT Publication No. WO 00/53211 and U.S. Pat. No. 5,981,568.

Various formulations of a TrkB agonist antibody may be used for administration. In some embodiments, the TrkB agonist antibody may be administered neat. In some embodiments, TrkB agonist antibody and a pharmaceutically acceptable excipient may be in various formulations. Pharmaceutically acceptable excipients are known in the art, and are relatively inert substances that facilitate administration of a pharmacologically effective substance. For example, an excipient can give form or consistency, or act as a diluent. Suitable excipients include but are not limited to stabilizing agents, wetting and emulsifying agents, salts for varying osmolarity, encapsulating agents, buffers, and skin penetration enhancers. Excipients as well as formulations for parenteral and nonparenteral drug delivery are set forth in Remington, The Science and Practice of Pharmacy 20th Ed. Mack Publishing (2000).

In some embodiments, these agents are formulated for administration by injection (e.g., intraperitoneally, intravenously, subcutaneously, intramuscularly, etc.). Accordingly, these agents can be combined with pharmaceutically acceptable vehicles such as saline, Ringer's solution, dextrose solution, and the like. The particular dosage regimen, i.e., dose, timing and repetition, will depend on the particular individual and that individual's medical history.

A TrkB agonist antibody can be administered using any suitable method, including by injection (e.g., intraperitoneally, intravenously, subcutaneously, intramuscularly, etc.). TrkB antibodies can also be administered via inhalation, as described herein. Generally, for administration of TrkB antibodies, an initial candidate dosage can be about 0.5-2 mg/kg. For the purpose of the present invention, a typical daily dosage might range from about any of 1 μg/kg to 3 μg/kg, to 30 μg/kg, to 300 μg/kg, to 3 mg/kg, to 30 mg/kg, to 100 mg/kg or more depending on the factors mentioned above. For example, dosage of about 1 μg/kg, about 10 μg/kg, about 100 μg/kg, about 500 μg/kg, about 1 mg/kg, about 2.5 mg/kg, about 5 mg/kg, about 10 mg/kg, and about 25 mg/kg may be used. For repeated administrations over several days or longer, depending on the condition, the treatment is sustained until a desired suppression of symptoms occurs or until sufficient therapeutic levels are achieved, for example, to increase CMAP area. An exemplary dosing regimen comprises administering an initial dose of about 2 mg/kg, followed by a weekly maintenance dose of about 1 mg/kg of the TrkB antibody, or followed by a maintenance dose of about 1 mg/kg every other week. However, other dosage regimens may be useful, depending on the pattern of pharmacokinetic decay that the practitioner wishes to achieve. For example, in some embodiments, dosing from one to four times a week is contemplated. In other embodiments dosing once a month or once every other month or every three months is contemplated. The progress of this therapy is easily monitored by conventional techniques and assays. The dosing regimen (including the TrkB agonist(s) used) can vary over time.

For the purpose of the present invention, the appropriate dosage of a TrkB agonist antibody will depend on the TrkB agonist antibody (or compositions thereof) employed, the type and severity of symptoms to be treated, whether the agent is administered for preventive or therapeutic purposes, previous therapy, the patient's clinical history and response to the agent, the patient's blood TrkB levels, the patient's synthesis and clearance rate for TrkB, the patient's clearance rate for the administered agent, and the discretion of the attending physician. Typically the clinician will administer a TrkB agonist antibody until a dosage is reached that achieves the desired result. Dose and/or frequency can vary over course of treatment. Empirical considerations, such as the half-life, generally will contribute to the determination of the dosage. For example, antibodies that are compatible with the human immune system, such as humanized antibodies or fully human antibodies, may be used to prolong half-life of the antibody and to prevent the antibody being attacked by the host's immune system. Frequency of administration may be determined and adjusted over the course of therapy, and is generally, but not necessarily, based on treatment and/or suppression and/or amelioration and/or delay of symptoms, e.g., weakness of foot muscles, weakness of lower leg muscles, weakness of the hand muscles, and muscle atrophy. Alternatively, sustained continuous release formulations of TrkB agonist antibodies may be appropriate. Various formulations and devices for achieving sustained release are known in the art.

In some embodiments, dosages for an agonist antibody may be determined empirically in individuals who have been given one or more administration(s) of an agonist antibody. Individuals are given incremental dosages of a TrkB agonist antibody. To assess efficacy, an indicator of the disease can be followed.

Administration of a TrkB agonist antibody in accordance with the method in the present invention can be continuous or intermittent, depending, for example, upon the recipient's physiological condition, whether the purpose of the administration is therapeutic or prophylactic, and other factors known to skilled practitioners. The administration of a TrkB agonist antibody may be essentially continuous over a preselected period of time or may be in a series of spaced doses.

In some embodiments, more than one agonist antibody may be present. At least one, at least two, at least three, at least four, at least five different, or more agonist antibody and/or peptide can be present. Generally, those TrkB agonist antibodies or peptides may have complementary activities that do not adversely affect each other. A TrkB agonist antibody can also be used in conjunction with other TrkB agonists or TrkB receptor agonists. For example, one or more of the following TrkB agonists may be used: an anti-sense molecule directed to a TrkB (including an anti-sense molecule directed to a nucleic acid encoding TrkB), a TrkB inhibitory compound, and a TrkB structural analog. A TrkB agonist antibody can also be used in conjunction with other agents that serve to enhance and/or complement the effectiveness of the agents.

Therapeutic formulations of the TrkB agonist antibody used in accordance with the present invention are prepared for storage by mixing an antibody or peptide having the desired degree of purity with optional pharmaceutically acceptable carriers, excipients or stabilizers (Remington, The Science and Practice of Pharmacy 20th Ed. Mack Publishing, 2000), in the form of lyophilized formulations or aqueous solutions. Acceptable carriers, excipients, or stabilizers are nontoxic to recipients at the dosages and concentrations employed, and may comprise buffers such as phosphate, citrate, and other organic acids; salts such as sodium chloride; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens, such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

Liposomes containing the TrkB agonist antibody are prepared by methods known in the art, such as described in Epstein, et al., Proc. Natl. Acad. Sci. USA 82:3688, 1985; Hwang, et al., Proc. Natl. Acad. Sci. USA 77:4030, 1980; and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

The active ingredients may also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles and nanocapsules) or in macroemulsions. Such techniques are disclosed in Remington, The Science and Practice of Pharmacy 20th Ed. Mack Publishing, 2000.

Sustained-release preparations may be prepared. Suitable examples of sustained-release preparations include semipermeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

The formulations to be used for in vivo administration must be sterile. This is readily accomplished by, for example, filtration through sterile filtration membranes. Therapeutic TrkB agonist antibody compositions are generally placed into a container having a sterile access port, for example, an intravenous solution bag or vial having a stopper pierceable by a hypodermic injection needle.

The compositions according to the present invention may be in unit dosage forms such as tablets, pills, capsules, powders, granules, solutions or suspensions, or suppositories, for oral, parenteral or rectal administration, or administration by inhalation or insufflation.

For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

Suitable surface-active agents include, in particular, non-ionic agents, such as polyoxyethylenesorbitans (e.g. Tween™ 20, 40, 60, 80 or 85) and other sorbitans (e.g. Span™20, 40, 60, 80 or 85). Compositions with a surface-active agent will conveniently comprise between 0.05 and 5% surface-active agent, and can be between 0.1 and 2.5%. It will be appreciated that other ingredients may be added, for example mannitol or other pharmaceutically acceptable vehicles, if necessary.

Suitable emulsions may be prepared using commercially available fat emulsions, such as Infralipid™, Liposyn™, Infonutrol™, Lipofundin™ and Lipiphysan™. The active ingredient may be either dissolved in a pre-mixed emulsion composition or alternatively it may be dissolved in an oil (e.g. soybean oil, safflower oil, cottonseed oil, sesame oil, corn oil or almond oil) and an emulsion formed upon mixing with a phospholipid (e.g. egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion can comprise fat droplets between 0.1 and 1.0 μm, particularly 0.1 and 0.5 μm, and have a pH in the range of 5.5 to 8.0.

The emulsion compositions can be those prepared by mixing a TrkB agonist antibody with Intralipid™ or the components thereof (soybean oil, egg phospholipids, glycerol and water).

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect. Compositions in preferably sterile pharmaceutically acceptable solvents may be nebulised by use of gases. Nebulised solutions may be breathed directly from the nebulising device or the nebulising device may be attached to a face mask, tent or intermittent positive pressure breathing machine. Solution, suspension or powder compositions may be administered, preferably orally or nasally, from devices which deliver the formulation in an appropriate manner.

### B. TrkB Agonist Antibody

The methods of the invention use a TrkB agonist antibody, which refers to an antibody that is able to bind to and activate a TrkB receptor and/or downstream pathway(s) mediated by the TrkB signaling function, such as elicitation of a cellular response to TrkB.

A TrkB agonist antibody should exhibit any one or more of the following characteristics: (a) bind to TrkB; (b) promote TrkB receptor dimerization; (c) improve nerve function; (e) promote increased CMAP area; (f) promote increased myelin sheath thickness; (g) improve axon morphology; and (h) promote neuron survival.

For purposes of this invention, the antibody preferably reacts with TrkB in a manner that promotes TrkB signaling function. In some embodiments, the TrkB agonist antibody specifically recognizes primate TrkB. In some embodiments, the TrkB agonist antibody binds primate and rodent TrkB.

The antibodies useful in the present invention can encompass monoclonal antibodies, polyclonal antibodies, antibody fragments (e.g., Fab, Fab′, F(ab′)₂, Fv, Fc, etc.), chimeric antibodies, bispecific antibodies, heteroconjugate antibodies, single chain (ScFv), mutants thereof, fusion proteins comprising an antibody portion (e.g., a domain antibody), humanized antibodies, and any other modified configuration of the immunoglobulin molecule that comprises an antigen recognition site of the required specificity, including glycosylation variants of antibodies, amino acid sequence variants of antibodies, and covalently modified antibodies. The antibodies may be murine, rat, human, or any other origin (including chimeric or humanized antibodies).

In some embodiments, the TrkB agonist antibody can be a monoclonal antibody. In some embodiments, the TrkB agonist antibody can be a humanized antibody. In other embodiments, the antibody can be a human antibody.

In some embodiments, the antibody comprises a modified constant region, such as a constant region that is immunologically inert, that is, having a reduced potential for provoking an immune response. In some embodiments, the constant region is modified as described in Eur. J. Immunol. (1999) 29:2613-2624; PCT Application No. PCT/GB99/01441; and/or UK Patent Application No. 9809951.8. The Fc can be human IgG₂ or human IgG₄. The Fc can be human IgG₂ containing the mutation A330P331 to S330S331 (IgG_{2Δa}), in which the amino acid residues are numbered with reference to the wild type IgG2 sequence. Eur. J. Immunol. (1999) 29:2613-2624. In some embodiments, the antibody comprises a constant region of IgG₄ comprising the following mutations (Armour et al., (2003) Molecular Immunology 40 585-593): E233F234L235 to P233V234A235 (IgG_{4Δc}), in which the numbering is with reference to wild type IgG4. In some embodiments, the Fc is human IgG₄ E233F234L235 to P233V234A235 with deletion G236 (IgG_{4Δb}). In another embodiment the Fc is any human IgG₄ Fc (IgG₄, IgG_{4Δb} or IgG_{4Δc}) containing hinge stabilizing mutation S228 to P228 (Aalberse et al., (2002) Immunology 105, 9-19). In another embodiment, the Fc can be aglycosylated Fc.

In some embodiments, the constant region is aglycosylated by mutating the oligosaccharide attachment residue (such as Asn297) and/or flanking residues that are part of the glycosylation recognition sequence in the constant region. In some embodiments, the constant region is aglycosylated for N-linked glycosylation enzymatically. The constant region may be aglycosylated for N-linked glycosylation enzymatically or by expression in a glycosylation deficient host cell.

The binding affinity (K_{D}) of a TrkB agonist antibody to TrkB (such as human TrkB) can be about 0.002 to about 200 nM. In some embodiments, the binding affinity is any of about 200 nM, about 100 nM, about 50 nM, about 10 nM, about 1 nM, about 500 pM, about 100 pM, about 60 pM, about 50 pM, about 20 pM, about 15 pM, about 10 pM, about 5 pM, or about 2 pM. In some embodiments, the binding affinity is less than any of about 250 nM, about 200 nM, about 100 nM, about 50 nM, about 10 nM, about 1 nM, about 500 pM, about 100 pM, about 50 pM, about 20 pM, about 10 pM, about 5 pM, or about 2 pM.

In some embodiments, the anti-TrkB agonist antibody has an EC₅₀ (half of the maximal effective concentration) of less than about any of 0.01 nM, 0.1 nM, 0.5 nM, 1 nM, 5 nM, 10 nM, 50 nM, or 100 nM in TrkB receptor (e.g., human TrkB) activation in vitro (e.g., assays described in US 2005/0209148; US 2007/0248611; PCT Pub. No. WO 2005/082401).

One way of determining binding affinity of antibodies to TrkB is by measuring binding affinity of monofunctional Fab fragments of the antibody. To obtain monofunctional Fab fragments, an antibody (for example, IgG) can be cleaved with papain or expressed recombinantly. The affinity of a TrkB Fab fragment of an antibody can be determined by surface plasmon resonance (Biacore3000™ surface plasmon resonance (SPR) system, Biacore, INC, Piscataway N.J.) equipped with pre-immobilized streptavidin sensor chips (SA) using HBS-EP running buffer (0.01M HEPES, pH 7.4, 0.15 NaCl, 3 mM EDTA, 0.005% v/v Surfactant P20). Biotinylated human TrkB (or any other TrkB) can be diluted into HBS-EP buffer to a concentration of less than 0.5 μg/mL and injected across the individual chip channels using variable contact times, to achieve two ranges of antigen density, either 50-200 response units (RU) for detailed kinetic studies or 800-1,000 RU for screening assays. Regeneration studies have shown that 25 mM NaOH in 25% v/v ethanol effectively removes the bound Fab while keeping the activity of TrkB on the chip for over 200 injections. Typically, serial dilutions (spanning concentrations of 0.1-10× estimated K_{D}) of purified Fab samples are injected for 1 min at 100 μL/minute and dissociation times of up to 2 hours are allowed. The concentrations of the Fab proteins are determined by ELISA and/or SDS-PAGE electrophoresis using a Fab of known concentration (as determined by amino acid analysis) as a standard. Kinetic association rates (kₒₙ) and dissociation rates (k_{off}) are obtained simultaneously by fitting the data globally to a 1:1 Langmuir binding model (Karlsson, R. Roos, H. Fagerstam, L. Petersson, B. (1994). Methods Enzymology 6. 99-110) using the BIAevaluation program. Equilibrium dissociation constant (K_{D}) values are calculated as k_{off}/kₒₙ. This protocol is suitable for use in determining binding affinity of an antibody to any TrkB, including human TrkB, TrkB of another mammalian (such as mouse TrkB, rat TrkB, primate TrkB), as well as different forms of TrkB (such as α and β form). Binding affinity of an antibody is generally measured at 25° C., but can also be measured at 37° C.

The TrkB agonist antibodies may be made by any method known in the art, including methods described in, for example, PCT Application Publication No. WO 2008/078179, herein incorporated by reference in its entirety. The route and schedule of immunization of the host animal are generally in keeping with established and conventional techniques for antibody stimulation and production, as further described herein. General techniques for production of human and mouse antibodies are known in the art and/or are described herein. A currently preferred method of making the antibodies comprises the immunization of TrkB⁻ knockout (TrkB −/−) animals. Generation of TrkB agonist antibodies are described in, for example, US 2007/0248611.

It is contemplated that any mammalian subject including humans or antibody producing cells therefrom can be manipulated to serve as the basis for production of mammalian, including human, hybridoma cell lines. Typically, the host animal is inoculated intraperitoneally, intramuscularly, orally, subcutaneously, intraplantar, and/or intradermally with an amount of immunogen, including as described herein.

Hybridomas can be prepared from the lymphocytes and immortalized myeloma cells using the general somatic cell hybridization technique of Kohler, B. and Milstein, C. (1975) Nature 256:495-497 or as modified by Buck, D. W., et al., In vitro, 18:377-381 (1982). Available myeloma lines, including but not limited to X63-Ag8.653 and those from the Salk Institute, Cell Distribution Center, San Diego, Calif., USA, may be used in the hybridization. Generally, the technique involves fusing myeloma cells and lymphoid cells using a fusogen such as polyethylene glycol, or by electrical means well known to those skilled in the art. After the fusion, the cells are separated from the fusion medium and grown in a selective growth medium, such as hypoxanthine-aminopterin-thymidine (HAT) medium, to eliminate unhybridized parent cells. Any of the media described herein, supplemented with or without serum, can be used for culturing hybridomas that secrete monoclonal antibodies. As another alternative to the cell fusion technique, EBV immortalized B cells may be used to produce the TrkB monoclonal antibodies of the subject invention. The hybridomas are expanded and subcloned, if desired, and supernatants are assayed for anti-immunogen activity by conventional immunoassay procedures (e.g., radioimmunoassay, enzyme immunoassay, or fluorescence immunoassay).

Hybridomas that may be used as source of antibodies encompass all derivatives, progeny cells of the parent hybridomas that produce monoclonal antibodies specific for TrkB, or a portion thereof.

Hybridomas that produce such antibodies may be grown in vitro or in vivo using known procedures. The monoclonal antibodies may be isolated from the culture media or body fluids, by conventional immunoglobulin purification procedures such as ammonium sulfate precipitation, gel electrophoresis, dialysis, chromatography, and ultrafiltration, if desired. Undesired activity, if present, can be removed, for example, by running the preparation over adsorbents made of the immunogen attached to a solid phase and eluting or releasing the desired antibodies off the immunogen. Immunization of a host animal with a human TrkB, or a fragment containing the target amino acid sequence conjugated to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are different alkyl groups, can yield a population of antibodies (e.g., monoclonal antibodies).

If desired, the TrkB agonist antibody (monoclonal or polyclonal) of interest may be sequenced and the polynucleotide sequence may then be cloned into a vector for expression or propagation. The sequence encoding the antibody of interest may be maintained in vector in a host cell and the host cell can then be expanded and frozen for future use. Production of recombinant monoclonal antibodies in cell culture can be carried out through cloning of antibody genes from B cells by means known in the art. See, e.g. Tiller et al. (2008) J. Immunol. Methods 329, 112; U.S. Pat. No. 7,314,622.

In an alternative, the polynucleotide sequence may be used for genetic manipulation to “humanize” the antibody or to improve the affinity, or other characteristics of the antibody. For example, the constant region may be engineered to more nearly resemble human constant regions to avoid immune response if the antibody is used in clinical trials and treatments in humans. It may be desirable to genetically manipulate the antibody sequence to obtain greater affinity to TrkB and greater efficacy in inhibiting TrkB. It will be apparent to one of skill in the art that one or more polynucleotide changes can be made to the TrkB agonist antibody and still maintain its binding ability to TrkB.

There are four general steps to humanize a monoclonal antibody. These are: (1) determining the nucleotide and predicted amino acid sequence of the starting antibody light and heavy variable domains (2) designing the humanized antibody, i.e., deciding which antibody framework region to use during the humanizing process (3) the actual humanizing methodologies/techniques and (4) the transfection and expression of the humanized antibody. See, for example, U.S. Pat. Nos. 4,816,567; 5,807,715; 5,866,692; 6,331,415; 5,530,101; 5,693,761; 5,693,762; 5,585,089; and 6,180,370.

A number of “humanized” antibody molecules comprising an antigen-binding site derived from a non-human immunoglobulin have been described, including chimeric antibodies having rodent or modified rodent V regions and their associated CDRs fused to human constant domains. See, for example, Winter et al. Nature 349:293-299, 1991, Lobuglio et al. Proc. Nat. Acad. Sci. USA 86:4220-4224, 1989, Shaw et al. J. Immunol. 138:4534-4538, 1987, and Brown et al. Cancer Res. 47:3577-3583, 1987. Other references describe rodent CDRs grafted into a human supporting framework region (FR) prior to fusion with an appropriate human antibody constant domain. See, for example, Riechmann et al. Nature 332:323-327, 1988, Verhoeyen et al. Science 239:1534-1536, 1988, and Jones et al. Nature 321:522-525, 1986. Another reference describes rodent CDRs supported by recombinantly engineered rodent framework regions. See, for example, European Patent Publication No. 0519596. These “humanized” molecules are designed to minimize unwanted immunological response toward rodent anti-human antibody molecules which limits the duration and effectiveness of therapeutic applications of those moieties in human recipients. For example, the antibody constant region can be engineered such that it is immunologically inert (e.g., does not trigger complement lysis). See, e.g. PCT Publication No. PCT/GB99/01441; UK Patent Application No. 9809951.8. Other methods of humanizing antibodies that may also be utilized are disclosed by Daugherty et al., Nucl. Acids Res. 19:2471-2476, 1991, and in U.S. Pat. Nos. 6,180,377; 6,054,297; 5,997,867; 5,866,692; 6,210,671; and 6,350,861; and in PCT Publication No. WO 01/27160.

In yet another alternative, fully human antibodies may be obtained by using commercially available mice that have been engineered to express specific human immunoglobulin proteins. Transgenic animals that are designed to produce a more desirable (e.g., fully human antibodies) or more robust immune response may also be used for generation of humanized or human antibodies. Examples of such technology are Xenomouse™ from Abgenix, Inc. (Fremont, Calif.) and HuMAb-Mouse® and TC Mouse™ from Medarex, Inc. (Princeton, N.J.).

In an alternative, antibodies may be made recombinantly and expressed using any method known in the art. In another alternative, antibodies may be made recombinantly by phage display technology. See, for example, U.S. Pat. Nos. 5,565,332; 5,580,717; 5,733,743; and 6,265,150; and Winter et al., Annu. Rev. Immunol. 12:433-455 (1994). Alternatively, the phage display technology (McCafferty et al., Nature 348:552-553 (1990)) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats; for review see, e.g., Johnson, Kevin S, and Chiswell, David J., Current Opinion in Structural Biology 3:564-571 (1993). Several sources of V-gene segments can be used for phage display. Clackson et al., Nature 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Mark et al., J. Mol. Biol. 222:581-597 (1991), or Griffith et al., EMBO J. 12:725-734 (1993). In a natural immune response, antibody genes accumulate mutations at a high rate (somatic hypermutation). Some of the changes introduced will confer higher affinity, and B cells displaying high-affinity surface immunoglobulin are preferentially replicated and differentiated during subsequent antigen challenge. This natural process can be mimicked by employing the technique known as “chain shuffling.” (Marks et al., Bio/Technol. 10:779-783 (1992)). In this method, the affinity of “primary” human antibodies obtained by phage display can be improved by sequentially replacing the heavy and light chain V region genes with repertoires of naturally occurring variants (repertoires) of V domain genes obtained from unimmunized donors. This technique allows the production of antibodies and antibody fragments with affinities in the pM-nM range. A strategy for making very large phage antibody repertoires (also known as “the mother-of-all libraries”) has been described by Waterhouse et al., Nucl. Acids Res. 21:2265-2266 (1993). Gene shuffling can also be used to derive human antibodies from rodent antibodies, where the human antibody has similar affinities and specificities to the starting rodent antibody. According to this method, which is also referred to as “epitope imprinting”, the heavy or light chain V domain gene of rodent antibodies obtained by phage display technique is replaced with a repertoire of human V domain genes, creating rodent-human chimeras. Selection on antigen results in isolation of human variable regions capable of restoring a functional antigen-binding site, i.e., the epitope governs (imprints) the choice of partner. When the process is repeated in order to replace the remaining rodent V domain, a human antibody is obtained (see PCT Publication No. WO 93/06213). Unlike traditional humanization of rodent antibodies by CDR grafting, this technique provides completely human antibodies, which have no framework or CDR residues of rodent origin.

It is apparent that although the above discussion pertains to humanized antibodies, the general principles discussed are applicable to customizing antibodies for use, for example, in dogs, cats, primate, equines and bovines. It is further apparent that one or more aspects of humanizing an antibody described herein may be combined, e.g., CDR grafting, framework mutation and CDR mutation.

Antibodies may be made recombinantly by first isolating the antibodies and antibody producing cells from host animals, obtaining the gene sequence, and using the gene sequence to express the antibody recombinantly in host cells (e.g., CHO cells). Another method which may be employed is to express the antibody sequence in plants (e.g., tobacco) or transgenic milk. Methods for expressing antibodies recombinantly in plants or milk have been disclosed. See, for example, Peeters, et al. Vaccine 19:2756 (2001); Lonberg, N. and D. Huszar Int. Rev. Immunol 13:65 (1995); and Pollock, et al., J Immunol Methods 231:147 (1999). Methods for making derivatives of antibodies, e.g., humanized, single chain, etc. are known in the art.

Immunoassays and flow cytometry sorting techniques such as fluorescence activated cell sorting (FACS) can also be employed to isolate antibodies that are specific for TrkB.

The antibodies can be bound to many different carriers. Carriers can be active and/or inert. Examples of well-known carriers include polypropylene, polystyrene, polyethylene, dextran, nylon, amylases, glass, natural and modified celluloses, polyacrylamides, agaroses and magnetite. The nature of the carrier can be either soluble or insoluble for purposes of the invention. Those skilled in the art will know of other suitable carriers for binding antibodies, or will be able to ascertain such, using routine experimentation. In some embodiments, the carrier comprises a moiety that targets the myocardium.

DNA encoding the monoclonal antibodies is readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into expression vectors (such as expression vectors disclosed in PCT Publication No. WO 87/04462), which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. See, e.g., PCT Publication No. WO 87/04462. The DNA also may be modified, for example, by substituting the coding sequence for human heavy and light chain constant domains in place of the homologous murine sequences, Morrison et al., Proc. Nat. Acad. Sci. 81:6851 (1984), or by covalently joining to the immunoglobulin coding sequence all or part of the coding sequence for a non-immunoglobulin polypeptide. In that manner, “chimeric” or “hybrid” antibodies are prepared that have the binding specificity of a TrkB monoclonal antibody herein.

TrkB agonist antibodies and polypeptides derived from antibodies can be identified or characterized using methods known in the art, whereby reduction, amelioration, or neutralization of TrkB biological activity is detected and/or measured. In some embodiments, a TrkB agonist antibody or polypeptide is identified by incubating a candidate agent with TrkB and monitoring binding and/or attendant reduction or neutralization of a biological activity of TrkB. The binding assay may be performed with purified TrkB polypeptide(s), or with cells naturally expressing, or transfected to express, TrkB polypeptide(s). In some embodiments, the binding assay is a competitive binding assay, where the ability of a candidate antibody to compete with a known TrkB agonist for TrkB binding is evaluated. The assay may be performed in various formats, including the ELISA format. In other embodiments, a TrkB agonist antibody is identified by incubating a candidate agent with TrkB and monitoring binding and attendant neuron survival in a Nodose assay.

Following initial identification, the activity of a candidate TrkB agonist antibody can be further confirmed and refined by bioassays, known to test the targeted biological activities. Alternatively, bioassays can be used to screen candidates directly.

TrkB agonist antibodies may be characterized using methods well known in the art. For example, one method is to identify the epitope to which it binds, or “epitope mapping.” There are many methods known in the art for mapping and characterizing the location of epitopes on proteins, including solving the crystal structure of an antibody-antigen complex, competition assays, gene fragment expression assays, and synthetic peptide-based assays, as described, for example, in Chapter 11 of Harlow and Lane, Using Antibodies, a Laboratory Manual, Cold Spring Harbor Laboratory Press, Cold Spring Harbor, N.Y., 1999. In an additional example, epitope mapping can be used to determine the sequence to which a TrkB agonist antibody binds. Epitope mapping is commercially available from various sources, for example, Pepscan Systems (Edelhertweg 15, 8219 PH Lelystad, The Netherlands). The epitope can be a linear epitope, i.e., contained in a single stretch of amino acids, or a conformational epitope formed by a three-dimensional interaction of amino acids that may not necessarily be contained in a single stretch. Peptides of varying lengths (e.g., at least 4-6 amino acids long) can be isolated or synthesized (e.g., recombinantly) and used for binding assays with a TrkB agonist antibody. In another example, the epitope to which the TrkB agonist antibody binds can be determined in a systematic screening by using overlapping peptides derived from the TrkB sequence and determining binding by the TrkB agonist antibody. According to the gene fragment expression assays, the open reading frame encoding TrkB is fragmented either randomly or by specific genetic constructions and the reactivity of the expressed fragments of TrkB with the antibody to be tested is determined. The gene fragments may, for example, be produced by PCR and then transcribed and translated into protein in vitro, in the presence of radioactive amino acids. The binding of the antibody to the radioactively labeled TrkB fragments is then determined by immunoprecipitation and gel electrophoresis. Certain epitopes can also be identified by using large libraries of random peptide sequences displayed on the surface of phage particles (phage libraries). Alternatively, a defined library of overlapping peptide fragments can be tested for binding to the test antibody in simple binding assays. In an additional example, mutagenesis of an antigen binding domain, domain swapping experiments and alanine scanning mutagenesis can be performed to identify residues required, sufficient, and/or necessary for epitope binding. For example, domain swapping experiments can be performed using a mutant TrkB in which various fragments of the TrkB polypeptide have been replaced (swapped) with sequences from TrkB from another species, or a closely related, but antigenically distinct protein (such as another member of the proprotein convertase family). By assessing binding of the antibody to the mutant TrkB, the importance of the particular TrkB fragment to antibody binding can be assessed.

Yet another method which can be used to characterize a TrkB agonist antibody is to use competition assays with other antibodies known to bind to the same antigen, i.e., various fragments on TrkB, to determine if the TrkB agonist antibody binds to the same epitope as other antibodies. Competition assays are well known to those of skill in the art.

An expression vector can be used to direct expression of a TrkB agonist antibody. One skilled in the art is familiar with administration of expression vectors to obtain expression of an exogenous protein in vivo. See, e.g., U.S. Patent Nos. 6,436,908; 6,413,942; and 6,376,471. Administration of expression vectors includes local or systemic administration, including injection, oral administration, particle gun or catheterized administration, and topical administration. In another embodiment, the expression vector is administered directly to the sympathetic trunk or ganglion, or into a coronary artery, atrium, ventrical, or pericardium.

Targeted delivery of therapeutic compositions containing an expression vector, or subgenomic polynucleotides can also be used. Receptor-mediated DNA delivery techniques are described in, for example, Findeis et al., Trends Biotechnol. (1993) 11:202; Chiou et al., Gene Therapeutics: Methods And Applications Of Direct Gene Transfer (J. A. Wolff, ed.) (1994); Wu et al., J. Biol. Chem. (1988) 263:621; Wu et al., J. Biol. Chem. (1994) 269:542; Zenke et al., Proc. Natl. Acad. Sci. USA (1990) 87:3655; Wu et al., J. Biol. Chem. (1991) 266:338. Therapeutic compositions containing a polynucleotide are administered in a range of about 100 ng to about 200 mg of DNA for local administration in a gene therapy protocol. Concentration ranges of about 500 ng to about 50 mg, about 1 μg to about 2 mg, about 5 μg to about 500 μg, and about 20 μg to about 100 μg of DNA can also be used during a gene therapy protocol. The therapeutic polynucleotides and polypeptides can be delivered using gene delivery vehicles. The gene delivery vehicle can be of viral or non-viral origin (see generally, Jolly, Cancer Gene Therapy (1994) 1:51; Kimura, Human Gene Therapy (1994) 5:845; Connelly, Human Gene Therapy (1995) 1:185; and Kaplitt, Nature Genetics (1994) 6:148). Expression of such coding sequences can be induced using endogenous mammalian or heterologous promoters. Expression of the coding sequence can be either constitutive or regulated.

Viral-based vectors for delivery of a desired polynucleotide and expression in a desired cell are well known in the art. Exemplary viral-based vehicles include, but are not limited to, recombinant retroviruses (see, e.g., PCT Publication Nos. WO 90/07936; WO 94/03622; WO 93/25698; WO 93/25234; WO 93/11230; WO 93/10218; WO 91/02805; U.S. Pat. Nos. 5,219,740 and 4,777,127; GB Patent No. 2,200,651; and EP Patent No. 0 345 242), alphavirus-based vectors (e.g., Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532)), and adeno-associated virus (AAV) vectors (see, e.g., PCT Publication Nos. WO 94/12649, WO 93/03769; WO 93/19191; WO 94/28938; WO 95/11984 and WO 95/00655). Administration of DNA linked to killed adenovirus as described in Curiel, Hum. Gene Ther. (1992) 3:147 can also be employed.

Non-viral delivery vehicles and methods can also be employed, including, but not limited to, polycationic condensed DNA linked or unlinked to killed adenovirus alone (see, e.g., Curiel, Hum. Gene Ther. (1992) 3:147); ligand-linked DNA (see, e.g., Wu, J. Biol. Chem. (1989) 264:16985); eukaryotic cell delivery vehicles cells (see, e.g., U.S. Pat. No. 5,814,482; PCT Publication Nos. WO 95/07994; WO 96/17072; WO 95/30763; and WO 97/42338) and nucleic charge neutralization or fusion with cell membranes. Naked DNA can also be employed. Exemplary naked DNA introduction methods are described in PCT Publication No. WO 90/11092 and U.S. Pat. No. 5,580,859. Liposomes that can act as gene delivery vehicles are described in U.S. Pat. No. 5,422,120; PCT Publication Nos. WO 95/13796; WO 94/23697; WO 91/14445; and EP 0524968. Additional approaches are described in Philip, Mol. Cell. Biol. (1994) 14:2411, and in Woffendin, Proc. Natl. Acad. Sci. (1994) 91:1581.

This invention encompasses compositions, including pharmaceutical compositions, comprising antibodies described herein or made by the methods and having the characteristics described herein. As used herein, compositions comprise one or more antibodies promote TrkB activity, and/or one or more polynucleotides comprising sequences encoding one or more these antibodies. These compositions may further comprise suitable excipients, such as pharmaceutically acceptable excipients including buffers, which are well known in the art.

The TrkB agonist antibodies of the invention are characterized by any (one or more) of the following characteristics: (a) bind to TrkB; (b) promote TrkB receptor dimerization; (c) improve nerve function; (e) promote increased CMAP area; (f) promote increased myelin sheath thickness; (g) improve axon morphology; and (h) promote neuron survival. Preferably, TrkB antibodies have two or more of these features. More preferably, the antibodies have three or more of the features. More preferably, the antibodies have four or more of the features. More preferably, the antibodies have five or more of the features. More preferably, the antibodies have six or more of the features. Most preferably, the antibodies have all seven characteristics.

Accordingly, the invention provides any of the following, or compositions (including pharmaceutical compositions) comprising any of the following: (a) an antibody having a partial light chain sequence of DIQMTQSPSSLSASVGDRVTITCX¹X²SX³X⁴VX⁵X⁶X⁷X⁸AWYQQKPGKAPKLLIYX⁹ASNL X¹⁰X¹¹GVPSRFSGSGSGTDX¹²TFTISSLQPEDIATYYCQX¹³FX¹⁴X¹⁵X¹⁶X¹⁷X¹⁸X¹⁹FGQG TKLEIK (SEQ ID NO: 20), wherein X¹ is R or H, X² is A or T, X³ is E, T, S or K, X⁴ is N, P, T, S or A, X⁵ is Y or T, X⁶ is S, R, L, Y, N or M, X⁷ is N or H, X⁸ is L, V or T, X⁹ is A or I, X¹⁰ is Q or A, X¹¹ is S or D, X¹² is F or Y, X¹³ is H, G, N, V, D or Q, X¹⁴ is W, Y, D, S, K, G or V, X¹⁵ is Y, K, V, W, G, Q, A, L, H, M, E, T or D, X¹⁶ is S, R or L, X¹⁷ is P, G or W, X¹⁸ is F, C or W, and X¹⁹ is T, G, I, K, V, L, A or W; (b) an antibody having a partial heavy chain sequence of QVQLVQSGAEVKKPGASVKVSCKASGYTFTNYX¹IX²WVRQAPGQGLEWMGX³IX⁴X⁵X⁶X⁷X⁸X⁹X¹⁰X¹¹X¹²X¹³X¹⁴KFKX¹⁵RVTMTRDTSTSTVYMELSSLRSEDTAVYYCARLLX¹⁶X¹⁷X¹⁸X¹⁹X²⁰X²¹X²²X²³AX²⁴X²⁵ X²⁶WGQGTTVTVSS (SEQ ID NO: 21), wherein X¹ is D or V, X² is I or L, X³ is Y or H, X⁴ is N, S or A, X⁵ is P or A, X⁶ is Y or A, X⁷ is N, Q, V or A, X⁸ is G, R, D, A or E, X⁹ is R or G, X¹⁰ is R, T, K or I, X¹¹ is E or K, X¹² is Y, E or A, X¹³ is N or A, X¹⁴ is E or A, X¹⁵ is G or Y, X¹⁶ is K, A or R, X¹⁷ is Y or A, X¹⁸ is R or A, X¹⁹ is R, C, A or P, X²⁰ is F, E, A, H, m or L, X²¹ is R, S, A, K, T or Q, X²² is Y, E, A or F, X²³ is Y, E, or A, X²⁴ is I, E or A, X²⁵ is D or H, and X²⁶ is Y, E or V; (c) an antibody having a partial light chain sequence of DIQMTQSPSSLSASVGDRVTITCX¹X²SEX³VYSNX⁴AWYQQKPGKAPKLLIYAASNLX⁵X⁶GVPSRFSGSGSGTDX⁷TFTISSLQPEDIATYYCQHFWX⁸SPFTFGQGTKLEIK (SEQ ID NO: 170), wherein X¹ is R or H, X² is A or T, X³ is N or P, X⁴ is L or V, X⁵ is Q or A, X⁶ is S or D; X⁷ is F or Y, and X⁸ is Y or G; (d) an antibody having a partial heavy chain sequence of QVQLVQSGAEVKKPGASVKVSCKASGYTFTNYX¹IX²WVRQAPGQGLEWMGYINPYN X³X⁴X⁵X⁶YNEKFKGRVTMTRDTSTSTVYMELSSLRSEDTAVYYCARLLKYRRFX⁷YYAID YWGQGTTVTVSS (SEQ ID NO: 171), wherein X¹ is D or V, X² is I or L, X³ is G, R or D, X⁴ is R or G, X⁵ is R or T, X⁶ is E or K, and X⁷ is R or S; (e) an antibody having a partial light chain sequence of DIQMTQSPSSLSASVGDRVTITCRASENVYSNLAWYQQKPGKAPKLLIYAASNLQSGV PSRFSGSGSGTDFTFTISSLQPEDIATYYCQHFWGSPFTFGQGTKLEIK (SEQ ID NO: 7); (f) an antibody having a partial heavy chain sequence of QVQLVQSGAEVKKPGASVKVSCKASGYTFTNYDIIWVRQAPGQGLEWMGYINPYNRR REYNEKFKGRVTMTRDTSTSTVYMELSSLRSEDTAVYYCARLLKYRRFRYYAIDYWGQ GTTVTVSS (SEQ ID NO: 8); (g) an antibody having a partial light chain sequence of DIQMTQSPSSLSASVGDRVTITCRASENVYSNLAWYQQKPGKAPKLLIYAASNLADGV PSRFSGSGSGTDFTFTISSLQPEDIATYYCQHFWYSPFTFGQGTKLEIK (SEQ ID NO: 9); (h) an antibody having a partial heavy chain sequence of QVQLVQSGAEVKKPGASVKVSCKASGYTFTNYDIIWVRQAPGQGLEWMGYINPYNDG TKYNEKFKGRVTMTRDTSTSTVYMELSSLRSEDTAVYYCARLLKYRRFSYYAIDYWGQ GTTVTVSS (SEQ ID NO: 10); (i) an antibody having a partial light chain sequence of DIQMTQSPSSLSASVGDRVTITCRTSENVYSNLAWYQQKPGKAPKLLIYAASNLQSGV PSRFSGSGSGTDFTFTISSLQPEDIATYYCQHFWGSPFTFGQGTKLEIK (SEQ ID NO: 11); (j) an antibody having a partial heavy chain sequence of QVQLVQSGAEVKKPGASVKVSCKASGYTFTNYDIIWVRQAPGQGLEWMGYINPYNDG TKYNEKFKGRVTMTRDTSTSTVYMELSSLRSEDTAVYYCARLLKYRRFRYYAIDYWGQ GTTVTVSS (SEQ ID NO: 12); (k) an antibody having a partial light chain sequence of DIQMTQSPSSLSASVGDRVTITCHASENVYSNLAWYQQKPGKAPKLLIYAASNLQSGV PSRFSGSGSGTDYTFTISSLQPEDIATYYCQHFWGSPFTFGQGTKLEIK (SEQ ID NO: 13); (l) an antibody having a partial light chain sequence of DIQMTQSPSSLSASVGDRVTITCRASEPVYSNVAWYQQKPGKAPKLLIYAASNLQSGV PSRFSGSGSGTDYTFTISSLQPEDIATYYCQHFWGSPFTFGQGTKLEIK (SEQ ID NO: 15); (m) an antibody having a partial heavy chain sequence of QVQLVQSGAEVKKPGASVKVSCKASGYTFTNYDIIWVRQAPGQGLEWMGYINPYNGR REYNEKFKGRVTMTRDTSTSTVYMELSSLRSEDTAVYYCARLLKYRRFRYYAIDYWGQ GTTVTVSS (SEQ ID NO: 16); (n) an antibody having a partial light chain sequence of DIQMTQSPSSLSASVGDRVTITCRTSENVYSNLAWYQQKPGKAPKLLIYAASNLQSGV PSRFSGSGSGTDYTFTISSLQPEDIATYYCQHFWGSPFTFGQGTKLEIK (SEQ ID NO: 173); and (o) an antibody having a partial light chain sequence of DIQMTQSPSSLSASVGDRVTITCRASENVYSNLAWYQQKPGKAPKLLIYAASNLQSGV PSRFSGSGSGTDYTFTISSLQPEDIATYYCQHFWYSPWTFGQGTKLEIK (SEQ ID NO: 176). Further provided are any of the following, or compositions (including pharmaceutical compositions) comprising any of the following: (a) an antibody having a partial light chain sequence of any of the light chain variable region sequences provided in Table 2; and (b) an antibody having a partial heavy chain sequence of any of the heavy chain variable region sequences provided in Table 2.

TABLE 2

| | | | |
|---|---|---|---|
| | Light Chain | Heavy Chain | T_{1/2} |
| mAb | Variable Region Sequence | Variable Region Sequence | (m) |

| | | | |
|---|---|---|---|
| RN | DIQMTQSPSSLSASVGDRVTITC | QVQLVQSGAEVKKPGASVKVSC | 4.5* |
| 1026A | RASENVYSNLAWYQQKPGKAP | KASGYTFTNYDIIWVRQAPGQG | |
| | KLLIYAASNLQSGVPSRFSGSG | LEWMGYINPYNRRREYNEKFKG | |
| | SGTDFTFTISSLQPEDIATYYCQ | RVTMTRDTSTSTVYMELSSLRS | |
| | HFWGSPFTFGQGTKLEIK | EDTAVYYCARLLKYRRFRYYAID | |
| | (SEQ ID NO: 7) | YWGQGTTVTVSS (SEQ ID NO: | |
| | | 8) | |
| T1- | DIQMTQSPSSLSASVGDRVTITC | QVQLVQSGAEVKKPGASVKVSC | 1.7 |
| HuC1 | RASENVYSNLAWYQQKPGKAP | KASGYTFTNYDIIWVRQAPGQG | |
| | KLLIYAASNLADGVPSRFSGSG | LEWMGYINPYNDGTKYNEKFKG | |
| | SGTDFTFTISSLQPEDIATYYCQ | RVTMTRDTSTSTVYMELSSLRS | |
| | HFWYSPFTFGQGTKLEIK | EDTAVYYCARLLKYRRFSYYAID | |
| | (SEQ ID NO: 9) | YWGQGTTVTVSS (SEQ ID NO: | |
| | | 10) | |
| C2 | DIQMTQSPSSLSASVGDRVTITC | QVQLVQSGAEVKKPGASVKVSC | 2.9 |
| | RTSENVYSNLAWYQQKPGKAP | KASGYTFTNYDIIWVRQAPGQG | |
| | KLLIYAASNLQSGVPSRFSGSG | LEWMGYINPYNDGTKYNEKFKG | |
| | SGTDYTFTISSLQPEDIATYYCQ | RVTMTRDTSTSTVYMELSSLRS | |
| | HFWGSPFTFGQGTKLEIK (SEQ | EDTAVYYCARLLKYRRFRYYAID | |
| | ID NO: 173) | YWGQGTTVTVSS (SEQ ID NO: | |
| | | 12) | |
| A2 | DIQMTQSPSSLSASVGDRVTITC | QVQLVQSGAEVKKPGASVKVSC | 6.0 |
| | RASENVYSNLAWYQQKPGKAP | KASGYTFTNYDIIWVRQAPGQG | |
| | KLLIYAASNLQSGVPSRFSGSG | LEWMGYINPYNDGTKYNEKFKG | |
| | SGTDYTFTISSLQPEDIATYYCQ | RVTMTRDTSTSTVYMELSSLRS | |
| | HFWYSPWTFGQGTKLEIK (SEQ | EDTAVYYCARLLKYRRFRYYAID | |
| | ID NO: 176) | YWGQGTTVTVSS (SEQ ID NO: | |
| | | 12) | |
| 4A6 | DIQMTQSPSSLSASVGDRVTITC | QVQLVQSGAEVKKPGASVKVSC | 13.2 |
| | HASENVYSNLAWYQQKPGKAP | KASGYTFTNYDIIWVRQAPGQG | |
| | KLLIYAASNLQSGVPSRFSGSG | LEWMGYINPYNRRREYNEKFKG | |
| | SGTDYTFTISSLQPEDIATYYCQ | RVTMTRDTSTSTVYMELSSLRS | |
| | HFWGSPFTFGQGTKLEIK | EDTAVYYCARLLKYRRFRYYAID | |
| | (SEQ ID NO: 13) | YWGQGTTVTVSS (SEQ ID NO: | |
| | | 8) | |
| 4B12 | DIQMTQSPSSLSASVGDRVTITC | QVQLVQSGAEVKKPGASVKVSC | 12.8 |
| | RASEPVYSNVAWYQQKPGKAP | KASGYTFTNYDIIWVRQAPGQG | |
| | KLLIYAASNLQSGVPSRFSGSG | LEWMGYINPYNGRREYNEKFKG | |
| | SGTDYTFTISSLQPEDIATYYCQ | RVTMTRDTSTSTVYMELSSLRS | |
| | HFWGSPFTFGQGTKLEIK | EDTAVYYCARLLKYRRFRYYAID | |
| | (SEQ ID NO: 15) | YWGQGTTVTVSS (SEQ ID NO: | |
| | | 16) | |
| 4B12-2 | DIQMTQSPSSLSASVGDRVTITC | QVQLVQSGAEVKKPGASVKVSC | 5.8 |
| | RASEPVYSNVAWYQQKPGKAP | KASGYTFTNYDIIWVRQAPGQG | |
| | KLLIYAASNLQSGVPSRFSGSG | LEWMGYINPYQGRREYNEKFKG | |
| | SGTDYTFTISSLQPEDIATYYCQ | RVTMTRDTSTSTVYMELSSLRS | |
| | HFWGSPFTFGQGTKLEIK | EDTAVYYCARLLKYRRFRYYAID | |
| | (SEQ ID NO: 15) | YWGQGTTVTVSS (SEQ ID NO: | |
| | | 29) | |
| 4B12-3 | DIQMTQSPSSLSASVGDRVTITC | QVQLVQSGAEVKKPGASVKVSC | 6.6 |
| | RASEPVYSNVAWYQQKPGKAP | KASGYTFTNYDIIWVRQAPGQG | |
| | KLLIYAASNLQSGVPSRFSGSG | LEWMGYINPYVGRREYNEKFKG | |
| | SGTDYTFTISSLQPEDIATYYCQ | RVTMTRDTSTSTVYMELSSLRS | |
| | HFWGSPFTFGQGTKLEIK | EDTAVYYCARLLKYRRFRYYAID | |
| | (SEQ ID NO: 15) | YWGQGTTVTVSS (SEQ ID NO: | |
| | | 30) | |
| 4B12-4 | DIQMTQSPSSLSASVGDRVTITC | QVQLVQSGAEVKKPGASVKVSC | 11.4 |
| | RASEPVYSNVAWYQQKPGKAP | KASGYTFTNYDIIWVRQAPGQG | |
| | KLLIYAASNLQSGVPSRFSGSG | LEWMGYINPYNARREYNEKFKG | |
| | SGTDYTFTISSLQPEDIATYYCQ | RVTMTRDTSTSTVYMELSSLRS | |
| | HFWGSPFTFGQGTKLEIK | EDTAVYYCARLLKYRRFRYYAID | |
| | (SEQ ID NO: 15) | YWGQGTTVTVSS (SEQ ID NO: | |
| | | 31) | |
| 4B12-5 | DIQMTQSPSSLSASVGDRVTITC | QVQLVQSGAEVKKPGASVKVSC | 16.1 |
| | RASEPVYSNVAWYQQKPGKAP | KASGYTFTNYDIIWVRQAPGQG | |
| | KLLIYAASNLQSGVPSRFSGSG | LEWMGYINPYNDRREYNEKFKG | |
| | SGTDYTFTISSLQPEDIATYYCQ | RVTMTRDTSTSTVYMELSSLRS | |
| | HFWGSPFTFGQGTKLEIK | EDTAVYYCARLLKYRRFRYYAID | |
| | (SEQ ID NO: 15) | YWGQGTTVTVSS (SEQ ID NO: | |
| | | 32) | |
| 4B12-6 | DIQMTQSPSSLSASVGDRVTITC | QVQLVQSGAEVKKPGASVKVSC | 3.7 |
| | RASEPVYSNVAWYQQKPGKAP | KASGYTFTNYDIIWVRQAPGQG | |
| | KLLIYAASNLQSGVPSRFSGSG | LEWMGYINPYNGGREYNEKFKG | |
| | SGTDYTFTISSLQPEDIATYYCQ | RVTMTRDTSTSTVYMELSSLRS | |
| | HFWGSPFTFGQGTKLEIK | EDTAVYYCARLLKYRRFRYYAID | |
| | (SEQ ID NO: 15) | YWGQGTTVTVSS (SEQ ID NO: | |
| | | 33) | |
| 4B12-7 | DIQMTQSPSSLSASVGDRVTITC | QVQLVQSGAEVKKPGASVKVSC | 7.6 |
| | RASEPVYSNVAWYQQKPGKAP | KASGYTFTNYDIIWVRQAPGQG | |
| | KLLIYAASNLQSGVPSRFSGSG | LEWMGYINPYNGRTEYNEKFKG | |
| | SGTDYTFTISSLQPEDIATYYCQ | RVTMTRDTSTSTVYMELSSLRS | |
| | HFWGSPFTFGQGTKLEIK | EDTAVYYCARLLKYRRFRYYAID | |
| | (SEQ ID NO: 15) | YWGQGTTVTVSS (SEQ ID NO: | |
| | | 34) | |
| 4B12-8 | DIQMTQSPSSLSASVGDRVTITC | QVQLVQSGAEVKKPGASVKVSC | 13.8 |
| | RASEPVYSNVAWYQQKPGKAP | KASGYTFTNYDIIWVRQAPGQG | |
| | KLLIYAASNLQSGVPSRFSGSG | LEWMGYINPYNGRRKYNEKFKG | |
| | SGTDYTFTISSLQPEDIATYYCQ | RVTMTRDTSTSTVYMELSSLRS | |
| | HFWGSPFTFGQGTKLEIK | EDTAVYYCARLLKYRRFRYYAID | |
| | (SEQ ID NO: 15) | YWGQGTTVTVSS (SEQ ID NO: | |
| | | 35) | |
| 4B12-13 | DIQMTQSPSSLSASVGDRVTITC | QVQLVQSGAEVKKPGASVKVSC | 13.7 |
| | RASEPVYSNVAWYQQKPGKAP | KASGYTFTNYDIIWVRQAPGQG | |
| | KLLIYAASNLQSGVPSRFSGSG | LEWMGYINPYNGRREYNAKFKG | |
| | SGTDYTFTISSLQPEDIATYYCQ | RVTMTRDTSTSTVYMELSSLRS | |
| | HFWGSPFTFGQGTKLEIK | EDTAVYYCARLLKYRRFRYYAID | |
| | (SEQ ID NO: 15) | YWGQGTTVTVSS (SEQ ID NO: | |
| | | 36) | |
| 4B12-14 | DIQMTQSPSSLSASVGDRVTITC | QVQLVQSGAEVKKPGASVKVSC | 3.2 |
| | RASEPVYSNVAWYQQKPGKAP | KASGYTFTNYDIIWVRQAPGQG | |
| | KLLIYAASNLQSGVPSRFSGSG | LEWMGYISPYNGRREYNEKFKG | |
| | SGTDYTFTISSLQPEDIATYYCQ | RVTMTRDTSTSTVYMELSSLRS | |
| | HFWGSPFTFGQGTKLEIK | EDTAVYYCARLLKYRRFRYYAID | |
| | (SEQ ID NO: 15) | YWGQGTTVTVSS (SEQ ID NO: | |
| | | 37) | |
| 4B12-15 | DIQMTQSPSSLSASVGDRVTITC | QVQLVQSGAEVKKPGASVKVSC | 2.7 |
| | RASEPVYSNVAWYQQKPGKAP | KASGYTFTNYDIIWVRQAPGQG | |
| | KLLIYAASNLQSGVPSRFSGSG | LEWMGYIAPYNGRREYNEKFKG | |
| | SGTDYTFTISSLQPEDIATYYCQ | RVTMTRDTSTSTVYMELSSLRS | |
| | HFWGSPFTFGQGTKLEIK | EDTAVYYCARLLKYRRFRYYAID | |
| | (SEQ ID NO: 15) | YWGQGTTVTVSS (SEQ ID NO: | |
| | | 38) | |
| 4B12-16 | DIQMTQSPSSLSASVGDRVTITC | QVQLVQSGAEVKKPGASVKVSC | 5.6 |
| | RASEPVYSNVAWYQQKPGKAP | KASGYTFTNYDIIWVRQAPGQG | |
| | KLLIYAASNLQSGVPSRFSGSG | LEWMGYINAYNGRREYNEKFKG | |
| | SGTDYTFTISSLQPEDIATYYCQ | RVTMTRDTSTSTVYMELSSLRS | |
| | HFWGSPFTFGQGTKLEIK | EDTAVYYCARLLKYRRFRYYAID | |
| | (SEQ ID NO: 15) | YWGQGTTVTVSS (SEQ ID NO: | |
| | | 39) | |
| 4B12-17 | DIQMTQSPSSLSASVGDRVTITC | QVQLVQSGAEVKKPGASVKVSC | 1.0 |
| | RASEPVYSNVAWYQQKPGKAP | KASGYTFTNYDIIWVRQAPGQG | |
| | KLLIYAASNLQSGVPSRFSGSG | LEWMGYINPANGRREYNEKFKG | |
| | SGTDYTFTISSLQPEDIATYYCQ | RVTMTRDTSTSTVYMELSSLRS | |
| | HFWGSPFTFGQGTKLEIK | EDTAVYYCARLLKYRRFRYYAID | |
| | (SEQ ID NO: 15) | YWGQGTTVTVSS (SEQ ID NO: | |
| | | 40) | |
| 4B12-18 | DIQMTQSPSSLSASVGDRVTITC | QVQLVQSGAEVKKPGASVKVSC | 3.8 |
| | RASEPVYSNVAWYQQKPGKAP | KASGYTFTNYDIIWVRQAPGQG | |
| | KLLIYAASNLQSGVPSRFSGSG | LEWMGYINPYAGRREYNEKFKG | |
| | SGTDYTFTISSLQPEDIATYYCQ | RVTMTRDTSTSTVYMELSSLRS | |
| | HFWGSPFTFGQGTKLEIK | EDTAVYYCARLLKYRRFRYYAID | |
| | (SEQ ID NO: 15) | YWGQGTTVTVSS (SEQ ID NO: | |
| | | 41) | |
| 4B12-19 | DIQMTQSPSSLSASVGDRVTITC | QVQLVQSGAEVKKPGASVKVSC | 13.1 |
| | RASEPVYSNVAWYQQKPGKAP | KASGYTFTNYDIIWVRQAPGQG | |
| | KLLIYAASNLQSGVPSRFSGSG | LEWMGYINPYNGRREANEKFKG | |
| | SGTDYTFTISSLQPEDIATYYCQ | RVTMTRDTSTSTVYMELSSLRS | |
| | HFWGSPFTFGQGTKLEIK | EDTAVYYCARLLKYRRFRYYAID | |
| | (SEQ ID NO: 15) | YWGQGTTVTVSS (SEQ ID NO: | |
| | | 42) | |
| 4B12-20 | DIQMTQSPSSLSASVGDRVTITC | QVQLVQSGAEVKKPGASVKVSC | 12.2 |
| | RASEPVYSNVAWYQQKPGKAP | KASGYTFTNYDIIWVRQAPGQG | |
| | KLLIYAASNLQSGVPSRFSGSG | LEWMGYINPYNGRREYAEKFKG | |
| | SGTDYTFTISSLQPEDIATYYCQ | RVTMTRDTSTSTVYMELSSLRS | |
| | HFWGSPFTFGQGTKLEIK | EDTAVYYCARLLKYRRFRYYAID | |
| | (SEQ ID NO: 15) | YWGQGTTVTVSS (SEQ ID NO: | |
| | | 43) | |
| 4B12-21 | DIQMTQSPSSLSASVGDRVTITC | QVQLVQSGAEVKKPGASVKVSC | .3 |
| | RASEPVYSNVAWYQQKPGKAP | KASGYTFTNYDIIWVRQAPGQG | |
| | KLLIYAASNLQSGVPSRFSGSG | LEWMGYINPYNGRREYNEKFKG | |
| | SGTDYTFTISSLQPEDIATYYCQ | RVTMTRDTSTSTVYMELSSLRS | |
| | HFWGSPFTFGQGTKLEIK | EDTAVYYCARLLAYRRFRYYAID | |
| | (SEQ ID NO: 15) | YWGQGTTVTVSS (SEQ ID NO: | |
| | | 44) | |
| 4B12-22 | DIQMTQSPSSLSASVGDRVTITC | QVQLVQSGAEVKKPGASVKVSC | 3.5 |
| | RASEPVYSNVAWYQQKPGKAP | KASGYTFTNYDIIWVRQAPGQG | |
| | KLLIYAASNLQSGVPSRFSGSG | LEWMGYINPYNGRREYNEKFKG | |
| | SGTDYTFTISSLQPEDIATYYCQ | RVTMTRDTSTSTVYMELSSLRS | |
| | HFWGSPFTFGQGTKLEIK | EDTAVYYCARLLKYARFRYYAID | |
| | (SEQ ID NO: 15) | YWGQGTTVTVSS (SEQ ID NO: | |
| | | 45) | |
| 4B12-23 | DIQMTQSPSSLSASVGDRVTITC | QVQLVQSGAEVKKPGASVKVSC | 10.8 |
| | RASEPVYSNVAWYQQKPGKAP | KASGYTFTNYDIIWVRQAPGQG | |
| | KLLIYAASNLQSGVPSRFSGSG | LEWMGYINPYNGRREYNEKFKG | |
| | SGTDYTFTISSLQPEDIATYYCQ | RVTMTRDTSTSTVYMELSSLRS | |
| | HFWGSPFTFGQGTKLEIK | EDTAVYYCARLLKYRAFRYYAID | |
| | (SEQ ID NO: 15) | YWGQGTTVTVSS (SEQ ID NO: | |
| | | 46) | |
| 4B12-24 | DIQMTQSPSSLSASVGDRVTITC | QVQLVQSGAEVKKPGASVKVSC | 6 |
| | RASEPVYSNVAWYQQKPGKAP | KASGYTFTNYDIIWVRQAPGQG | |
| | KLLIYAASNLQSGVPSRFSGSG | LEWMGYINPYNGRREYNEKFKG | |
| | SGTDYTFTISSLQPEDIATYYCQ | RVTMTRDTSTSTVYMELSSLRS | |
| | HFWGSPFTFGQGTKLEIK | EDTAVYYCARLLKYRRFSYYAID | |
| | (SEQ ID NO: 15) | YWGQGTTVTVSS (SEQ ID NO: | |
| | | 47) | |
| 4B12-25 | DIQMTQSPSSLSASVGDRVTITC | QVQLVQSGAEVKKPGASVKVSC | 1.1 |
| | RASEPVYSNVAWYQQKPGKAP | KASGYTFTNYDIIWVRQAPGQG | |
| | KLLIYAASNLQSGVPSRFSGSG | LEWMGYINPYNGRREYNEKFKG | |
| | SGTDYTFTISSLQPEDIATYYCQ | RVTMTRDTSTSTVYMELSSLRS | |
| | HFWGSPFTFGQGTKLEIK | EDTAVYYCARLLRYRRFRYYAID | |
| | (SEQ ID NO: 15) | YWGQGTTVTVSS (SEQ ID NO: | |
| | | 48) | |
| 4B12-26 | DIQMTQSPSSLSASVGDRVTITC | QVQLVQSGAEVKKPGASVKVSC | 8.1 |
| | RASEPVYSNVAWYQQKPGKAP | KASGYTFTNYDIIWVRQAPGQG | |
| | KLLIYAASNLQSGVPSRFSGSG | LEWMGYINPYNGRREYNEKFKG | |
| | SGTDYTFTISSLQPEDIATYYCQ | RVTMTRDTSTSTVYMELSSLRS | |
| | HFWGSPFTFGQGTKLEIK | EDTAVYYCARLLKYRRFAYYAID | |
| | (SEQ ID NO: 15) | YWGQGTTVTVSS (SEQ ID NO: | |
| | | 49) | |
| 4B12-27 | DIQMTQSPSSLSASVGDRVTITC | QVQLVQSGAEVKKPGASVKVSC | 0.5 |
| | RASEPVYSNVAWYQQKPGKAP | KASGYTFTNYDIIWVRQAPGQG | |
| | KLLIYAASNLQSGVPSRFSGSG | LEWMGYINPYNGRREYNEKFKG | |
| | SGTDYTFTISSLQPEDIATYYCQ | RVTMTRDTSTSTVYMELSSLRS | |
| | HFWGSPFTFGQGTKLEIK | EDTAVYYCARLLKARRFRYYAI | |
| | (SEQ ID NO: 15) | DYWGQGTTVTVSS (SEQ ID NO: | |
| | | 50) | |
| 4B12-28 | DIQMTQSPSSLSASVGDRVTITC | QVQLVQSGAEVKKPGASVKVSC | 5.4 |
| | RASEPVYSNVAWYQQKPGKAP | KASGYTFTNYDIIWVRQAPGQG | |
| | KLLIYAASNLQSGVPSRFSGSG | LEWMGYINPYNGRREYNEKFKG | |
| | SGTDYTFTISSLQPEDIATYYCQ | RVTMTRDTSTSTVYMELSSLRS | |
| | HFWGSPFTFGQGTKLEIK | EDTAVYYCARLLKYRRERYYAI | |
| | (SEQ ID NO: 15) | DYWGQGTTVTVSS (SEQ ID NO: | |
| | | 51) | |
| 4B12-29 | DIQMTQSPSSLSASVGDRVTITC | QVQLVQSGAEVKKPGASVKVSC | 5.6 |
| | RASEPVYSNVAWYQQKPGKAP | KASGYTFTNYDIIWVRQAPGQG | |
| | KLLIYAASNLQSGVPSRFSGSG | LEWMGYINPYNGRREYNEKFKG | |
| | SGTDYTFTISSLQPEDIATYYCQ | RVTMTRDTSTSTVYMELSSLRS | |
| | HFWGSPFTFGQGTKLEIK | EDTAVYYCARLLKYRRARYYAI | |
| | (SEQ ID NO: 15) | DYWGQGTTVTVSS (SEQ ID NO: | |
| | | 52) | |
| 4B12-30 | DIQMTQSPSSLSASVGDRVTITC | QVQLVQSGAEVKKPGASVKVSC | ND |
| | RASEPVYSNVAWYQQKPGKAP | KASGYTFTNYDIIWVRQAPGQG | |
| | KLLIYAASNLQSGVPSRFSGSG | LEWMGYINPYNGRREYNEKFKG | |
| | SGTDYTFTISSLQPEDIATYYCQ | RVTMTRDTSTSTVYMELSSLRS | |
| | HFWGSPFTFGQGTKLEIK | EDTAVYYCARLLKYRRFRAYAI | |
| | (SEQ ID NO: 15) | DYWGQGTTVTVSS (SEQ ID NO: | |
| | | 53) | |
| 4B12-31 | DIQMTQSPSSLSASVGDRVTITC | QVQLVQSGAEVKKPGASVKVSC | ND |
| | RASEPVYSNVAWYQQKPGKAP | KASGYTFTNYDIIWVRQAPGQG | |
| | KLLIYAASNLQSGVPSRFSGSG | LEWMGYINPYNGRREYNEKFKG | |
| | SGTDYTFTISSLQPEDIATYYCQ | RVTMTRDTSTSTVYMELSSLRS | |
| | HFWGSPFTFGQGTKLEIK | EDTAVYYCARLLKYRRFRYAAI | |
| | (SEQ ID NO: 15) | DYWGQGTTVTVSS (SEQ ID NO: | |
| | | 54) | |
| A6-9 | DIQMTQSPSSLSASVGDRVTITC | QVQLVQSGAEVKKPGASVKVSC | 11.8 |
| | HASENVYSNLAWYQQKPGKAP | KASGYTFTNYDIIWVRQAPGQG | |
| | KLLIYAASNLQSGVPSRFSGSG | LEWMGYINPYNDRRRYNEKFKG | |
| | SGTDYTFTISSLQPEDIATYYCQ | RVTMTRDTSTSTVYMELSSLRS | |
| | HFWGSPFTFGQGTKLEIK | EDTAVYYCARLLKYRRFRYYAID | |
| | (SEQ ID NO: 13) | YWGQGTTVTVSS (SEQ ID NO: | |
| | | 55) | |
| A6-10 | DIQMTQSPSSLSASVGDRVTITC | QVQLVQSGAEVKKPGASVKVSC | 4.0 |
| | HASENVYSNLAWYQQKPGKAP | KASGYTFTNYDIIWVRQAPGQG | |
| | KLLIYAASNLQSGVPSRFSGSG | LEWMGYINPYNRGREYNEKFKG | |
| | SGTDYTFTISSLQPEDIATYYCQ | RVTMTRDTSTSTVYMELSSLRS | |
| | HFWGSPFTFGQGTKLEIK | EDTAVYYCARLLKYRRFRYYAID | |
| | (SEQ ID NO: 13) | YWGQGTTVTVSS (SEQ ID NO: | |
| | | 56) | |
| A6-11 | DIQMTQSPSSLSASVGDRVTITC | QVQLVQSGAEVKKPGASVKVSC | 9.9 |
| | HASENVYSNLAWYQQKPGKAP | KASGYTFTNYDIIWVRQAPGQG | |
| | KLLIYAASNLQSGVPSRFSGSG | LEWMGYINPYNRRTEYNEKFKG | |
| | SGTDYTFTISSLQPEDIATYYCQ | RVTMTRDTSTSTVYMELSSLRS | |
| | HFWGSPFTFGQGTKLEIK | EDTAVYYCARLLKYRRFRYYAID | |
| | (SEQ ID NO: 13) | YWGQGTTVTVSS (SEQ ID NO: | |
| | | 57) | |
| 4A6-12 | DIQMTQSPSSLSASVGDRVTITC | QVQLVQSGAEVKKPGASVKVSC | 74.2 |
| | HASENVYSNLAWYQQKPGKAP | KASGYTFTNYDIIWVRQAPGQG | |
| | KLLIYAASNLQSGVPSRFSGSG | LEWMGYINPYNRRRKYNEKFKG | |
| | SGTDYTFTISSLQPEDIATYYCQ | RVTMTRDTSTSTVYMELSSLRS | |
| | HFWGSPFTFGQGTKLEIK | EDTAVYYCARLLKYRRFRYYAID | |
| | (SEQ ID NO: 13) | YWGQGTTVTVSS (SEQ ID NO: | |
| | | 58) | |
| 1-A11 | DIQMTQSPSSLSASVGDRVTITC | QVQLVQSGAEVKKPGASVKVSC | 1.08 |
| | RASENVYSNLAWYQQKPGKAP | KASGYTFTNYVILWVRQAPGQG | |
| | KLLIYAASNLQSGVPSRFSGSG | LEWMGYINPYNDGTKYNEKFKG | |
| | SGTDFTFTISSLQPEDIATYYCQ | RVTMTRDTSTSTVYMELSSLRS | |
| | HFWYSPFTFGQGTKLEIK (SEQ | EDTAVYYCARLLKYRRFSYYAID | |
| | ID NO: 59) | YWGQGTTVTVSS (SEQ ID NO: | |
| | | 60) | |
| 1-A6 | DIQMTQSPSSLSASVGDRVTITC | QVQLVQSGAEVKKPGASVKVSC | 0.42 |
| | RASENVYSNLAWYQQKPGKAP | KASGYTFTNYVILWVRQAPGQG | |
| | KLLIYAASNLQSGVPSRFSGSG | LEWMGYINPYNDGTKYNEKFKG | |
| | SGTDFTFTISSLQPEDIATYYCQ | RVTMTRDTSTSTVYMELSSLRS | |
| | HFWKSPFTFGQGTKLEIK (SEQ | EDTAVYYCARLLKYRRHSYYAI | |
| | ID NO: 61) | DYWGQGTTVTVSS (SEQ ID NO: | |
| | | 62) | |
| 1-B10 | DIQMTQSPSSLSASVGDRVTITC | QVQLVQSGAEVKKPGASVKVSC | 0.55 |
| | RASENVYSNLAWYQQKPGKAP | KASGYTFTNYVILWVRQAPGQG | |
| | KLLIYAASNLQSGVPSRFSGSG | LEWMGYINPYNDGTKYNEKFKG | |
| | SGTDFTFTISSLQPEDIATYYCQ | RVTMTRDTSTSTVYMELSSLRS | |
| | GFWYSPFTFGQGTKLEIK (SEQ | EDTAVYYCARLLKYRRFSFYAID | |
| | ID NO: 63) | YWGQGTTVTVSS (SEQ ID NO: | |
| | | 64) | |
| 1-B11 | DIQMTQSPSSLSASVGDRVTITC | QVQLVQSGAEVKKPGASVKVSC | 0.72 |
| | RASENVYSNLAWYQQKPGKAP | KASGYTFTNYVILWVRQAPGQG | |
| | KLLIYAASNLQSGVPSRFSGSG | LEWMGYINPYNDGTKYNEKFKG | |
| | SGTDFTFTISSLQPEDIATYYCQ | RVTMTRDTSTSTVYMELSSLRS | |
| | NFWYSPFTFGQGTKLEIK (SEQ | EDTAVYYCARLLKYRRFSYYAID | |
| | ID NO: 65) | YWGQGTTVTVSS (SEQ ID NO: | |
| | | 60) | |
| 1-D3 | DIQMTQSPSSLSASVGDRVTITC | QVQLVQSGAEVKKPGASVKVSC | 0.92 |
| | RASENVYSNLAWYQQKPGKAP | KASGYTFTNYVILWVRQAPGQG | |
| | KLLIYAASNLQSGVPSRFSGSG | LEWMGYINPYNDGTKYNEKFKG | |
| | SGTDFTFTISSLQPEDIATYYCQ | RVTMTRDTSTSTVYMELSSLRS | |
| | HFWYSPFTFGQGTKLEIK (SEQ | EDTAVYYCARLLKYRRFSYYAID | |
| | ID NO: 59) | EWGQGTTVTVSS (SEQ ID NO: | |
| | | 66) | |
| 1-E9 | DIQMTQSPSSLSASVGDRVTITC | QVQLVQSGAEVKKPGASVKVSC | 1.04 |
| | RASENVYSNLAWYQQKPGKAP | KASGYTFTNYVILWVRQAPGQG | |
| | KLLIYAASNLQSGVPSRFSGSG | LEWMGYINPYNDGTKYNEKFKG | |
| | SGTDFTFTISSLQPEDIATYYCQ | RVTMTRDTSTSTVYMELSSLRS | |
| | HFWYSPFTFGQGTKLEIK (SEQ | EDTAVYYCARLLKYRRMSYYAI | |
| | ID NO: 59) | DYWGQGTTVTVSS (SEQ ID NO: | |
| | | 67) | |
| 1-F1 | DIQMTQSPSSLSASVGDRVTITC | QVQLVQSGAEVKKPGASVKVSC | 0.66 |
| | RASENVYSNLAWYQQKPGKAP | KASGYTFTNYVILWVRQAPGQG | |
| | KLLIYAASNLQSGVPSRFSGSG | LEWMGYINPYNDGTKYNEKFKG | |
| | SGTDFTFTISSLQPEDIATYYCQ | RVTMTRDTSTSTVYMELSSLRS | |
| | HFWVSPFTFGQGTKLEIK (SEQ | EDTAVYYCARLLKYRRFKYYAID | |
| | ID NO: 68) | YWGQGTTVTVSS (SEQ ID NO: | |
| | | 69) | |
| 1-G12 | DIQMTQSPSSLSASVGDRVTITC | QVQLVQSGAEVKKPGASVKVSC | 0.4 |
| | RASENVYSNLAWYQQKPGKAP | KASGYTFTNYVILWVRQAPGQG | |
| | KLLIYAASNLQSGVPSRFSGSG | LEWMGYINPYNDGTKYNEKFKG | |
| | SGTDFTFTISSLQPEDIATYYCQ | RVTMTRDTSTSTVYMELSSLRS | |
| | HFWYSPFGFGQGTKLEIK (SEQ | EDTAVYYCARLLKYRRFSYYAID | |
| | ID NO: 70) | YWGQGTTVTVSS (SEQ ID NO: | |
| | | 60) | |
| 1-H6 | DIQMTQSPSSLSASVGDRVTITC | QVQLVQSGAEVKKPGASVKVSC | 0.62 |
| | RASENVYSNLAWYQQKPGKAP | KASGYTFTNYVILWVRQAPGQG | |
| | KLLIYAASNLQSGVPSRFSGSG | LEWMGYINPYNDGTKYNEKFKG | |
| | SGTDFTFTISSLQPEDIATYYCQ | RVTMTRDTSTSTVYMELSSLRS | |
| | HFWWSPFTFGQGTKLEIK (SEQ | EDTAVYYCARLLKYRRFSYYAID | |
| | ID NO: 71) | YWGQGTTVTVSS (SEQ ID NO: | |
| | | 60) | |
| 2-A2 | DIQMTQSPSSLSASVGDRVTITC | QVQLVQSGAEVKKPGASVKVSC | 0.57 |
| | RASENVYSNLAWYQQKPGKAP | KASGYTFTNYVILWVRQAPGQG | |
| | KLLIYAASNLQSGVPSRFSGSG | LEWMGYINPYNDGTKYNEKFKG | |
| | SGTDFTFTISSLQPEDIATYYCQ | RVTMTRDTSTSTVYMELSSLRS | |
| | HFWYSPFTFGQGTKLEIK (SEQ | EDTAVYYCARLLKYRRLSYYAID | |
| | ID NO: 59) | YWGQGTTVTVSS (SEQ ID NO: | |
| | | 72) | |
| 2-B1 | DIQMTQSPSSLSASVGDRVTITC | QVQLVQSGAEVKKPGASVKVSC | 0.32 |
| | RASENVYSNLAWYQQKPGKAP | KASGYTFTNYVILWVRQAPGQG | |
| | KLLIYAASNLQSGVPSRFSGSG | LEWMGYINPYNDGTKYNEKFKG | |
| | SGTDFTFTISSLQPEDIATYYCQ | RVTMTRDTSTSTVYMELSSLRS | |
| | NFWYSPFTFGQGTKLEIK (SEQ | EDTAVYYCARLLKYRRFSYYAID | |
| | ID NO: 73) | YWGQGTTVTVSS (SEQ ID NO: | |
| | | 60) | |
| 2-F6 | DIQMTQSPSSLSASVGDRVTITC | QVQLVQSGAEVKKPGASVKVSC | 0.23 |
| | RASENVYSNLAWYQQKPGKAP | KASGYTFTNYVILWVRQAPGQG | |
| | KLLIYAASNLQSGVPSRFSGSG | LEWMGYINPYNDGTKYNEKFKG | |
| | SGTDFTFTISSLQPEDIATYYCQ | RVTMTRDTSTSTVYMELSSLRS | |
| | HFWYSPFTFGQGTKLEIK (SEQ | EDTAVYYCARLLKYRRFRYYAID | |
| | ID NO: 59) | YWGQGTTVTVSS (SEQ ID NO: | |
| | | 74) | |
| 2-F10 | DIQMTQSPSSLSASVGDRVTITC | QVQLVQSGAEVKKPGASVKVSC | 0.48 |
| | RASENVYSNLAWYQQKPGKAP | KASGYTFTNYVILWVRQAPGQG | |
| | KLLIYAASNLQSGVPSRFSGSG | LEWMGYINPYNDGTKYNEKFKG | |
| | SGTDFTFTISSLQPEDIATYYCQ | RVTMTRDTSTSTVYMELSSLRS | |
| | VFWYSPFTFGQGTKLEIK (SEQ | EDTAVYYCARLLKYRRFSYYAID | |
| | ID NO: 75) | YWGQGTTVTVSS (SEQ ID NO: | |
| | | 60) | |
| 2-G12 | DIQMTQSPSSLSASVGDRVTITC | QVQLVQSGAEVKKPGASVKVSC | 0.3 |
| | RASENVYSNLAWYQQKPGKAP | KASGYTFTNYVILWVRQAPGQG | |
| | KLLIYAASNLQSGVPSRFSGSG | LEWMGYINPYNDGTKYNEKFKG | |
| | SGTDFTFTISSLQPEDIATYYCQ | RVTMTRDTSTSTVYMELSSLRS | |
| | HFWYRPFTFGQGTKLEIK (SEQ | EDTAVYYCARLLKYRRFRYYAID | |
| | ID NO: 76) | YWGQGTTVTVSS (SEQ ID NO: | |
| | | 74) | |
| 3-B11 | DIQMTQSPSSLSASVGDRVTITC | QVQLVQSGAEVKKPGASVKVSC | 0.87 |
| | RASENVYSNLAWYQQKPGKAP | KASGYTFTNYVILWVRQAPGQG | |
| | KLLIYAASNLQSGVPSRFSGSG | LEWMGYINPYNDGTKYNEKFKG | |
| | SGTDFTFTISSLQPEDIATYYCQ | RVTMTRDTSTSTVYMELSSLRS | |
| | HFWYSGFTFGQGTKLEIK (SEQ | EDTAVYYCARLLKYRRFRYYAID | |
| | ID NO: 77) | YWGQGTTVTVSS (SEQ ID NO: | |
| | | 74) | |
| 3-C12 | DIQMTQSPSSLSASVGDRVTITC | QVQLVQSGAEVKKPGASVKVSC | 0.84 |
| | RASENVYSNLAWYQQKPGKAP | KASGYTFTNYVILWVRQAPGQG | |
| | KLLIYAASNLQSGVPSRFSGSG | LEWMGYINPYNDGTKYNEKFKG | |
| | SGTDFTFTISSLQPEDIATYYCQ | RVTMTRDTSTSTVYMELSSLRS | |
| | HFWGSPFTFGQGTKLEIK (SEQ | EDTAVYYCARLLKYRRFSYYAID | |
| | ID NO: 7) | YWGQGTTVTVSS (SEQ ID NO: | |
| | | 60) | |
| 3-F2 | DIQMTQSPSSLSASVGDRVTITC | QVQLVQSGAEVKKPGASVKVSC | 1.38 |
| | RASENVYSNLAWYQQKPGKAP | KASGYTFTNYVILWVRQAPGQG | |
| | KLLIYAASNLQSGVPSRFSGSG | LEWMGYINPYNDGTKYNEKFKG | |
| | SGTDFTFTISSLQPEDIATYYCQ | RVTMTRDTSTSTVYMELSSLRS | |
| | HFWYSPFIFGQGTKLEIK (SEQ | EDTAVYYCARLLKYRRFSYYAID | |
| | ID NO: 79) | YWGQGTTVTVSS (SEQ ID NO: | |
| | | 60) | |
| 3-F7 | DIQMTQSPSSLSASVGDRVTITC | QVQLVQSGAEVKKPGASVKVSC | 1.01 |
| | RASENVYSNLAWYQQKPGKAP | KASGYTFTNYVILWVRQAPGQG | |
| | KLLIYAASNLQSGVPSRFSGSG | LEWMGYINPYNDGTKYNEKFKG | |
| | SGTDFTFTISSLQPEDIATYYCQ | RVTMTRDTSTSTVYMELSSLRS | |
| | HFWKSPFTFGQGTKLEIK (SEQ | EDTAVYYCARLLKYRRFRYYAID | |
| | ID NO: 61) | YWGQGTTVTVSS (SEQ ID NO: | |
| | | 74) | |
| 3-F10 | DIQMTQSPSSLSASVGDRVTITC | QVQLVQSGAEVKKPGASVKVSC | 0.79 |
| | RASENVYSNLAWYQQKPGKAP | KASGYTFTNYVILWVRQAPGQG | |
| | KLLIYAASNLQSGVPSRFSGSG | LEWMGYINPYNDGTKYNEKFKG | |
| | SGTDFTFTISSLQPEDIATYYCQ | RVTMTRDTSTSTVYMELSSLRS | |
| | HFWYSPFKFGQGTKLEIK (SEQ | EDTAVYYCARLLKYRRFSYYAID | |
| | ID NO: 81) | YWGQGTTVTVSS (SEQ ID NO: | |
| | | 60) | |
| 3-F11 | DIQMTQSPSSLSASVGDRVTITC | QVQLVQSGAEVKKPGASVKVSC | 1.16 |
| | RASENVYSNLAWYQQKPGKAP | KASGYTFTNYVILWVRQAPGQG | |
| | KLLIYAASNLQSGVPSRFSGSG | LEWMGYINPYNDGTKYNEKFKG | |
| | SGTDFTFTISSLQPEDIATYYCQ | RVTMTRDTSTSTVYMELSSLRS | |
| | HFWYSPFTFGQGTKLEIK (SEQ | EDTAVYYCARLLKYRRFRYYAID | |
| | ID NO: 59) | YWGQGTTVTVSS (SEQ ID NO: | |
| | | 74) | |
| 3-G5 | DIQMTQSPSSLSASVGDRVTITC | QVQLVQSGAEVKKPGASVKVSC | 1.21 |
| | RASENVYSNLAWYQQKPGKAP | KASGYTFTNYVILWVRQAPGQG | |
| | KLLIYAASNLQSGVPSRFSGSG | LEWMGYINPYNDGTKYNEKFKG | |
| | SGTDFTFTISSLQPEDIATYYCQ | RVTMTRDTSTSTVYMELSSLRS | |
| | NFWYSPFTFGQGTKLEIK (SEQ | EDTAVYYCARLLKYRRFSYYAID | |
| | ID NO: 73) | YWGQGTTVTVSS (SEQ ID NO: | |
| | | 60) | |
| 3-G7 | DIQMTQSPSSLSASVGDRVTITC | QVQLVQSGAEVKKPGASVKVSC | 0.92 |
| | RASENVYSNLAWYQQKPGKAP | KASGYTFTNYVILWVRQAPGQG | |
| | KLLIYAASNLQSGVPSRFSGSG | LEWMGYINPYNDGTKYNEKFKG | |
| | SGTDFTFTISSLQPEDIATYYCQ | RVTMTRDTSTSTVYMELSSLRS | |
| | NFWYSPFTFGQGTKLEIK (SEQ | EDTAVYYCARLLKYRRFSYYAE | |
| | ID NO: 73) | DYWGQGTTVTVSS (SEQ ID NO: | |
| | | 82) | |
| 3-H8 | DIQMTQSPSSLSASVGDRVTITC | QVQLVQSGAEVKKPGASVKVSC | 0.81 |
| | RASENVYSNLAWYQQKPGKAP | KASGYTFTNYVILWVRQAPGQG | |
| | KLLIYAASNLQSGVPSRFSGSG | LEWMGYINPYNDGTKYNEKFKG | |
| | SGTDFTFTISSLQPEDIATYYCQ | RVTMTRDTSTSTVYMELSSLRS | |
| | HFWYSPFVFGQGTKLEIK (SEQ | EDTAVYYCARLLKYRRFRYYAID | |
| | ID NO: 83) | YWGQGTTVTVSS (SEQ ID NO: | |
| | | 74) | |
| 4-A1 | DIQMTQSPSSLSASVGDRVTITC | QVQLVQSGAEVKKPGASVKVSC | 0.93 |
| | RASENVYSNLAWYQQKPGKAP | KASGYTFTNYVILWVRQAPGQG | |
| | KLLIYAASNLQSGVPSRFSGSG | LEWMGYINPYNDGTKYNEKFKG | |
| | SGTDFTFTISSLQPEDIATYYCQ | RVTMTRDTSTSTVYMELSSLRS | |
| | HFYYSPFTFGQGTKLEIK (SEQ | EDTAVYYCARLLKYRRFSYYAID | |
| | ID NO: 84) | YWGQGTTVTVSS (SEQ ID NO: | |
| | | 60) | |
| 4-B2 | DIQMTQSPSSLSASVGDRVTITC | QVQLVQSGAEVKKPGASVKVSC | 1.33 |
| | RASENVYSNLAWYQQKPGKAP | KASGYTFTNYVILWVRQAPGQG | |
| | KLLIYAASNLQSGVPSRFSGSG | LEWMGYINPYNDGTKYNEKFKG | |
| | SGTDFTFTISSLQPEDIATYYCQ | RVTMTRDTSTSTVYMELSSLRS | |
| | HFWGSPFTFGQGTKLEIK (SEQ | EDTAVYYCARLLKYRRFRYYAID | |
| | ID NO: 7) | YWGQGTTVTVSS (SEQ ID NO: | |
| | | 74) | |
| 4-C7 | DIQMTQSPSSLSASVGDRVTITC | QVQLVQSGAEVKKPGASVKVSC | 1.4 |
| | RASENVYSNLAWYQQKPGKAP | KASGYTFTNYVILWVRQAPGQG | |
| | KLLIYAASNLQSGVPSRFSGSG | LEWMGYINPYNDGTKYNEKFKG | |
| | SGTDFTFTISSLQPEDIATYYCQ | RVTMTRDTSTSTVYMELSSLRS | |
| | HFWGSPFTFGQGTKLEIK (SEQ | EDTAVYYCARLLKYRRFSYYAID | |
| | ID NO: 7) | YWGQGTTVTVSS (SEQ ID NO: | |
| | | 60) | |
| 4-D4 | DIQMTQSPSSLSASVGDRVTITC | QVQLVQSGAEVKKPGASVKVSC | 1.24 |
| | RASENVYSNLAWYQQKPGKAP | KASGYTFTNYVILWVRQAPGQG | |
| | KLLIYAASNLQSGVPSRFSGSG | LEWMGYINPYNDGTKYNEKFKG | |
| | SGTDFTFTISSLQPEDIATYYCQ | RVTMTRDTSTSTVYMELSSLRS | |
| | HFWYSPFTFGQGTKLEIK (SEQ | EDTAVYYCARLLKYRRFRYYAID | |
| | ID NO: 59) | YWGQGTTVTVSS (SEQ ID NO: | |
| | | 74) | |
| 4-E1 | DIQMTQSPSSLSASVGDRVTITC | QVQLVQSGAEVKKPGASVKVSC | 0.71 |
| | RASENVYSNLAWYQQKPGKAP | KASGYTFTNYVILWVRQAPGQG | |
| | KLLIYAASNLQSGVPSRFSGSG | LEWMGYINPYNDGTKYNEKFKG | |
| | SGTDFTFTISSLQPEDIATYYCQ | RVTMTRDTSTSTVYMELSSLRS | |
| | HFWGSPFTFGQGTKLEIK (SEQ | EDTAVYYCARLLKYRAFSYYAID | |
| | ID NO: 7) | YWGQGTTVTVSS (SEQ ID NO: | |
| | | 68) | |
| 4-F10 | DIQMTQSPSSLSASVGDRVTITC | QVQLVQSGAEVKKPGASVKVSC | 1.03 |
| | RASENVYSNLAWYQQKPGKAP | KASGYTFTNYVILWVRQAPGQG | |
| | KLLIYAASNLQSGVPSRFSGSG | LEWMGYINPYNDGTKYNEKFKG | |
| | SGTDFTFTISSLQPEDIATYYCQ | RVTMTRDTSTSTVYMELSSLRS | |
| | QFWYSPFTFGQGTKLEIK (SEQ | EDTAVYYCARLLKYRRFSYYAID | |
| | ID NO: 86) | YWGQGTTVTVSS (SEQ ID NO: | |
| | | 60) | |
| 5-B1 | DIQMTQSPSSLSASVGDRVTITC | QVQLVQSGAEVKKPGASVKVSC | 1.36 |
| | RASENVYSNLAWYQQKPGKAP | KASGYTFTNYVILWVRQAPGQG | |
| | KLLIYAASNLQSGVPSRFSGSG | LEWMGYINPYNDGTKYNEKFKG | |
| | SGTDFTFTISSLQPEDIATYYCQ | RVTMTRDTSTSTVYMELSSLRS | |
| | HFWYSPFTFGQGTKLEIK (SEQ | EDTAVYYCARLLKYRRFTYYAIH | |
| | ID NO: 59) | YWGQGTTVTVSS (SEQ ID NO: | |
| | | 87) | |
| 5-B2 | DIQMTQSPSSLSASVGDRVTITC | QVQLVQSGAEVKKPGASVKVSC | 1.55 |
| | RASENVYSNLAWYQQKPGKAP | KASGYTFTNYVILWVRQAPGQG | |
| | KLLIYAASNLQSGVPSRFSGSG | LEWMGYINPYNDGTKYNEKFKG | |
| | SGTDFTFTISSLQPEDIATYYCQ | RVTMTRDTSTSTVYMELSSLRS | |
| | HFDYSPFTFGQGTKLEIK (SEQ | EDTAVYYCARLLKYRRFSYYAID | |
| | ID NO: 88) | YWGQGTTVTVSS (SEQ ID NO: | |
| | | 60) | |
| 5-B8 | DIQMTQSPSSLSASVGDRVTITC | QVQLVQSGAEVKKPGASVKVSC | 1.82 |
| | RASENVYSNLAWYQQKPGKAP | KASGYTFTNYVILWVRQAPGQG | |
| | KLLIYAASNLQSGVPSRFSGSG | LEWMGYINPYNDGTKYNEKFKG | |
| | SGTDFTFTISSLQPEDIATYYCQ | RVTMTRDTSTSTVYMELSSLRS | |
| | QFWYSPFTFGQGTKLEIK (SEQ | EDTAVYYCARLLKYRRFRYYAID | |
| | ID NO: 86) | YWGQGTTVTVSS (SEQ ID NO: | |
| | | 74) | |
| 5-D1 | DIQMTQSPSSLSASVGDRVTITC | QVQLVQSGAEVKKPGASVKVSC | 1.61 |
| | RASENVYSNLAWYQQKPGKAP | KASGYTFTNYVILWVRQAPGQG | |
| | KLLIYAASNLQSGVPSRFSGSG | LEWMGYINPYNDGTKYNEKFKG | |
| | SGTDFTFTISSLQPEDIATYYCQ | RVTMTRDTSTSTVYMELSSLRS | |
| | NFWYSPFTFGQGTKLEIK (SEQ | EDTAVYYCARLLKYRRFRYYAID | |
| | ID NO: 73) | VWGQGTTVTVSS (SEQ ID NO: | |
| | | 90) | |
| 5-E2 | DIQMTQSPSSLSASVGDRVTITC | QVQLVQSGAEVKKPGASVKVSC | 1.66 |
| | RASENVYSNLAWYQQKPGKAP | KASGYTFTNYVILWVRQAPGQG | |
| | KLLIYAASNLQSGVPSRFSGSG | LEWMGYINPYNDGTKYNEKFKG | |
| | SGTDFTFTISSLQPEDIATYYCQ | RVTMTRDTSTSTVYMELSSLRS | |
| | HFWYSPFTFGQGTKLEIK (SEQ | EDTAVYYCARLLKYRRFKYYAID | |
| | ID NO: 59) | YWGQGTTVTVSS (SEQ ID NO: | |
| | | 68) | |
| 5-F1 | DIQMTQSPSSLSASVGDRVTITC | QVQLVQSGAEVKKPGASVKVSC | 1.6 |
| | RASENVYSNLAWYQQKPGKAP | KASGYTFTNYVILWVRQAPGQG | |
| | KLLIYAASNLQSGVPSRFSGSG | LEWMGYINPYNDGTKYNEKFKG | |
| | SGTDFTFTISSLQPEDIATYYCQ | RVTMTRDTSTSTVYMELSSLRS | |
| | HFWYSPFLFGQGTKLEIK (SEQ | EDTAVYYCARLLKYRRFKYYAID | |
| | ID NO: 91) | YWGQGTTVTVSS (SEQ ID NO: | |
| | | 68) | |
| 5-G4 | DIQMTQSPSSLSASVGDRVTITC | QVQLVQSGAEVKKPGASVKVSC | 1.35 |
| | RASENVYSNLAWYQQKPGKAP | KASGYTFTNYVILWVRQAPGQG | |
| | KLLIYAASNLQSGVPSRFSGSG | LEWMGYINPYNDGTKYNEKFKG | |
| | SGTDFTFTISSLQPEDIATYYCQ | RVTMTRDTSTSTVYMELSSLRS | |
| | HFWGSPFTFGQGTKLEIK (SEQ | EDTAVYYCARLLKYRRFSYYAID | |
| | ID NO: 7) | YWGQGTTVTVSS (SEQ ID NO: | |
| | | 60) | |
| 6-A6 | DIQMTQSPSSLSASVGDRVTITC | QVQLVQSGAEVKKPGASVKVSC | 1.59 |
| | RASENVYSNLAWYQQKPGKAP | KASGYTFTNYVILWVRQAPGQG | |
| | KLLIYAASNLQSGVPSRFSGSG | LEWMGYINPYNDGTKYNEKFKG | |
| | SGTDFTFTISSLQPEDIATYYCQ | RVTMTRDTSTSTVYMELSSLRS | |
| | HFWYSPFVFGQGTKLEIK (SEQ | EDTAVYYCARLLKYRRFKYYAID | |
| | ID NO: 83) | YWGQGTTVTVSS (SEQ ID NO: | |
| | | 68) | |
| 6-B4 | DIQMTQSPSSLSASVGDRVTITC | QVQLVQSGAEVKKPGASVKVSC | 1.55 |
| | RASENVYSNLAWYQQKPGKAP | KASGYTFTNYVILWVRQAPGQG | |
| | KLLIYAASNLQSGVPSRFSGSG | LEWMGYINPYNDGTKYNEKFKG | |
| | SGTDFTFTISSLQPEDIATYYCQ | RVTMTRDTSTSTVYMELSSLRS | |
| | HFWYSPFTFGQGTKLEIK (SEQ | EDTAVYYCARLLKYRRFQYYAI | |
| | ID NO: 59) | DYWGQGTTVTVSS (SEQ ID NO: | |
| | | 92) | |
| 6-B11 | DIQMTQSPSSLSASVGDRVTITC | QVQLVQSGAEVKKPGASVKVSC | 1.44 |
| | RASENVYSNLAWYQQKPGKAP | KASGYTFTNYVILWVRQAPGQG | |
| | KLLIYAASNLQSGVPSRFSGSG | LEWMGYINPYNDGTKYNEKFKG | |
| | SGTDFTFTISSLQPEDIATYYCQ | RVTMTRDTSTSTVYMELSSLRS | |
| | HFWQSPFTFGQGTKLEIK (SEQ | EDTAVYYCARLLKYRRFSYYAID | |
| | ID NO: 93) | YWGQGTTVTVSS (SEQ ID NO: | |
| | | 60) | |
| 6-C10 | DIQMTQSPSSLSASVGDRVTITC | QVQLVQSGAEVKKPGASVKVSC | 1.55 |
| | RASENVYSNLAWYQQKPGKAP | KASGYTFTNYVILWVRQAPGQG | |
| | KLLIYAASNLQSGVPSRFSGSG | LEWMGYINPYNDGTKYNEKFKG | |
| | SGTDFTFTISSLQPEDIATYYCQ | RVTMTRDTSTSTVYMELSSLRS | |
| | HFWYSWFTFGQGTKLEIK (SEQ | EDTAVYYCARLLKYRRFRYYAID | |
| | ID NO: 94) | YWGQGTTVTVSS (SEQ ID NO: | |
| | | 74) | |
| 6-D12 | DIQMTQSPSSLSASVGDRVTITC | QVQLVQSGAEVKKPGASVKVSC | 1.7 |
| | RASENVYSNLAWYQQKPGKAP | KASGYTFTNYVILWVRQAPGQG | |
| | KLLIYAASNLQSGVPSRFSGSG | LEWMGYINPYNDGTKYNEKFKG | |
| | SGTDFTFTISSLQPEDIATYYCQ | RVTMTRDTSTSTVYMELSSLRS | |
| | HFWASPFTFGQGTKLEIK (SEQ | EDTAVYYCARLLKYRRFRYYAID | |
| | ID NO: 95) | YWGQGTTVTVSS (SEQ ID NO: | |
| | | 74) | |
| 6-E10 | DIQMTQSPSSLSASVGDRVTITC | QVQLVQSGAEVKKPGASVKVSC | 1.3 |
| | RASENVYSNLAWYQQKPGKAP | KASGYTFTNYVILWVRQAPGQG | |
| | KLLIYAASNLQSGVPSRFSGSG | LEWMGYINPYNDGTKYNEKFKG | |
| | SGTDFTFTISSLQPEDIATYYCQ | RVTMTRDTSTSTVYMELSSLRS | |
| | HFSLSPFTFGQGTKLEIK (SEQ | EDTAVYYCARLLKYRPFSYYAID | |
| | ID NO: 96) | YWGQGTTVTVSS (SEQ ID NO: | |
| | | 97) | |
| 6-H12 | DIQMTQSPSSLSASVGDRVTITC | QVQLVQSGAEVKKPGASVKVSC | 1.54 |
| | RASENVYSNLAWYQQKPGKAP | KASGYTFTNYVILWVRQAPGQG | |
| | KLLIYAASNLQSGVPSRFSGSG | LEWMGYINPYNDGTKYNEKFKG | |
| | SGTDFTFTISSLQPEDIATYYCQ | RVTMTRDTSTSTVYMELSSLRS | |
| | HFWHSPFTFGQGTKLEIK (SEQ | EDTAVYYCARLLKYRRFSYYAID | |
| | ID NO: 98) | YWGQGTTVTVSS (SEQ ID NO: | |
| | | 60) | |
| 7-B1 | DIQMTQSPSSLSASVGDRVTITC | QVQLVQSGAEVKKPGASVKVSC | 0.81 |
| | RASENVYSNLAWYQQKPGKAP | KASGYTFTNYVILWVRQAPGQG | |
| | KLLIYAASNLQSGVPSRFSGSG | LEWMGYINPYNDGTKYNEKFKG | |
| | SGTDFTFTISSLQPEDIATYYCQ | RVTMTRDTSTSTVYMELSSLRS | |
| | HFWYSPFKFGQGTKLEIK (SEQ | EDTAVYYCARLLKYRRFRYYAID | |
| | ID NO: 81) | YWGQGTTVTVSS (SEQ ID NO: | |
| | | 74) | |
| 7-B8 | DIQMTQSPSSLSASVGDRVTITC | QVQLVQSGAEVKKPGASVKVSC | 1.18 |
| | RASENVYSNLAWYQQKPGKAP | KASGYTFTNYVILWVRQAPGQG | |
| | KLLIYAASNLQSGVPSRFSGSG | LEWMGYINPYNDGTKYNEKFKG | |
| | SGTDFTFTISSLQPEDIATYYCQ | RVTMTRDTSTSTVYMELSSLRS | |
| | HFWYSPCTFGQGTKLEIK (SEQ | EDTAVYYCARLLKYRRFRYYAID | |
| | ID NO: 99) | YWGQGTTVTVSS (SEQ ID NO: | |
| | | 74) | |
| 7-B9 | DIQMTQSPSSLSASVGDRVTITC | QVQLVQSGAEVKKPGASVKVSC | 1.79 |
| | RASENVYSNLAWYQQKPGKAP | KASGYTFTNYVILWVRQAPGQG | |
| | KLLIYAASNLQSGVPSRFSGSG | LEWMGYINPYNDGTKYNEKFKG | |
| | SGTDFTFTISSLQPEDIATYYCQ | RVTMTRDTSTSTVYMELSSLRS | |
| | HFWYSPWTFGQGTKLEIK (SEQ | EDTAVYYCARLLKYRRFSYYAID | |
| | ID NO: 100) | VWGQGTTVTVSS (SEQ ID NO: | |
| | | 101) | |
| 7-C11 | DIQMTQSPSSLSASVGDRVTITC | QVQLVQSGAEVKKPGASVKVSC | 1.07 |
| | RASENVYSNLAWYQQKPGKAP | KASGYTFTNYVILWVRQAPGQG | |
| | KLLIYAASNLQSGVPSRFSGSG | LEWMGYINPYNDGTKYNEKFKG | |
| | SGTDFTFTISSLQPEDIATYYCQ | RVTMTRDTSTSTVYMELSSLRS | |
| | HFKYSPFTFGQGTKLEIK (SEQ | EDTAVYYCARLLKYRRFSYYAID | |
| | ID NO: 102) | YWGQGTTVTVSS (SEQ ID NO: | |
| | | 60) | |
| 7-D1 | DIQMTQSPSSLSASVGDRVTITC | QVQLVQSGAEVKKPGASVKVSC | 0.7 |
| | RASENVYSNLAWYQQKPGKAP | KASGYTFTNYVILWVRQAPGQG | |
| | KLLIYAASNLQSGVPSRFSGSG | LEWMGYINPYNDGTKYNEKFKG | |
| | SGTDFTFTISSLQPEDIATYYCQ | RVTMTRDTSTSTVYMELSSLRS | |
| | HFWYLPFTFGQGTKLEIK (SEQ | EDTAVYYCARLLKYRRFSYYAID | |
| | ID NO: 103) | VWGQGTTVTVSS (SEQ ID NO: | |
| | | 101) | |
| 7-F1 | DIQMTQSPSSLSASVGDRVTITC | QVQLVQSGAEVKKPGASVKVSC | 0.54 |
| | RASENVYSNLAWYQQKPGKAP | KASGYTFTNYVILWVRQAPGQG | |
| | KLLIYAASNLQSGVPSRFSGSG | LEWMGYINPYNDGTKYNEKFKG | |
| | SGTDFTFTISSLQPEDIATYYCQ | RVTMTRDTSTSTVYMELSSLRS | |
| | NFWYSPFTFGQGTKLEIK (SEQ | EDTAVYYCARLLKYRRFRYYAID | |
| | ID NO: 73) | YWGQGTTVTVSS (SEQ ID NO: | |
| | | 74) | |
| 8-B2 | DIQMTQSPSSLSASVGDRVTITC | QVQLVQSGAEVKKPGASVKVSC | 1.46 |
| | RASENVYSNLAWYQQKPGKAP | KASGYTFTNYVILWVRQAPGQG | |
| | KLLIYAASNLQSGVPSRFSGSG | LEWMGYINPYNDGTKYNEKFKG | |
| | SGTDFTFTISSLQPEDIATYYCQ | RVTMTRDTSTSTVYMELSSLRS | |
| | HFYYSPFTFGQGTKLEIK (SEQ | EDTAVYYCARLLKYRRFRYYAID | |
| | ID NO: 84) | YWGQGTTVTVSS (SEQ ID NO: | |
| | | 74) | |
| 8-C8 | DIQMTQSPSSLSASVGDRVTITC | QVQLVQSGAEVKKPGASVKVSC | 1.78 |
| | RASENVYSNLAWYQQKPGKAP | KASGYTFTNYVILWVRQAPGQG | |
| | KLLIYAASNLQSGVPSRFSGSG | LEWMGYINPYNDGTKYNEKFKG | |
| | SGTDFTFTISSLQPEDIATYYCQ | RVTMTRDTSTSTVYMELSSLRS | |
| | HFWYSPFGFGQGTKLEIK (SEQ | EDTAVYYCARLLKYRRFRYYAID | |
| | ID NO: 70) | YWGQGTTVTVSS (SEQ ID NO: | |
| | | 74) | |
| 8-D2 | DIQMTQSPSSLSASVGDRVTITC | QVQLVQSGAEVKKPGASVKVSC | 1.81 |
| | RASENVYSNLAWYQQKPGKAP | KASGYTFTNYVILWVRQAPGQG | |
| | KLLIYAASNLQSGVPSRFSGSG | LEWMGYINPYNDGTKYNEKFKG | |
| | SGTDFTFTISSLQPEDIATYYCQ | RVTMTRDTSTSTVYMELSSLRS | |
| | NFWYSPFTFGQGTKLEIK (SEQ | EDTAVYYCARLLKYRRFSYYAA | |
| | ID NO: 73) | DYWGQGTTVTVSS (SEQ ID NO: | |
| | | 105) | |
| 8-G2 | DIQMTQSPSSLSASVGDRVTITC | QVQLVQSGAEVKKPGASVKVSC | 1.88 |
| | RASENVYSNLAWYQQKPGKAP | KASGYTFTNYVILWVRQAPGQG | |
| | KLLIYAASNLQSGVPSRFSGSG | LEWMGYINPYNDGTKYNEKFKG | |
| | SGTDFTFTISSLQPEDIATYYCQ | RVTMTRDTSTSTVYMELSSLRS | |
| | HFGYSPFIFGQGTKLEIK (SEQ | EDTAVYYCARLLKYRRFRFYAID | |
| | ID NO: 106) | YWGQGTTVTVSS (SEQ ID NO: | |
| | | 107) | |
| 8-H7 | DIQMTQSPSSLSASVGDRVTITC | QVQLVQSGAEVKKPGASVKVSC | 1.18 |
| | RASENVYSNLAWYQQKPGKAP | KASGYTFTNYVILWVRQAPGQG | |
| | KLLIYAASNLQSGVPSRFSGSG | LEWMGYINPYNDGTKYNEKFKG | |
| | SGTDFTFTISSLQPEDIATYYCQ | RVTMTRDTSTSTVYMELSSLRS | |
| | HFVYSPFTFGQGTKLEIK (SEQ | EDTAVYYCARLLKYRRFSFYAID | |
| | ID NO: 108) | YWGQGTTVTVSS (SEQ ID NO: | |
| | | 64) | |
| 9-D6 | DIQMTQSPSSLSASVGDRVTITC | QVQLVQSGAEVKKPGASVKVSC | 1.45 |
| | RASENVYSNLAWYQQKPGKAP | KASGYTFTNYVILWVRQAPGQG | |
| | KLLIYAASNLQSGVPSRFSGSG | LEWMGYINPYNDGTKYNEKFKG | |
| | SGTDFTFTISSLQPEDIATYYCQ | RVTMTRDTSTSTVYMELSSLRS | |
| | HFSYSPFTFGQGTKLEIK (SEQ | EDTAVYYCARLLKYRRFSYYAID | |
| | ID NO: 78) | VWGQGTTVTVSS (SEQ ID NO: | |
| | | 101) | |
| 9-D9 | DIQMTQSPSSLSASVGDRVTITC | QVQLVQSGAEVKKPGASVKVSC | 1.54 |
| | RASENVYSNLAWYQQKPGKAP | KASGYTFTNYVILWVRQAPGQG | |
| | KLLIYAASNLQSGVPSRFSGSG | LEWMGYINPYNDGTKYNEKFKG | |
| | SGTDFTFTISSLQPEDIATYYCQ | RVTMTRDTSTSTVYMELSSLRS | |
| | HFWYSPFAFGQGTKLEIK (SEQ | EDTAVYYCARLLKYRRFRYYAID | |
| | ID NO: 109) | YWGQGTTVTVSS (SEQ ID NO: | |
| | | 74) | |
| 9-D11 | DIQMTQSPSSLSASVGDRVTITC | QVQLVQSGAEVKKPGASVKVSC | 1.55 |
| | RASENVYSNLAWYQQKPGKAP | KASGYTFTNYVILWVRQAPGQG | |
| | KLLIYAASNLQSGVPSRFSGSG | LEWMGYINPYNDGTKYNEKFKG | |
| | SGTDFTFTISSLQPEDIATYYCQ | RVTMTRDTSTSTVYMELSSLRS | |
| | HFWMSPFTFGQGTKLEIK (SEQ | EDTAVYYCARLLKYRRFKYYAID | |
| | ID NO: 110) | YWGQGTTVTVSS (SEQ ID NO: | |
| | | 68) | |
| 9-G8 | DIQMTQSPSSLSASVGDRVTITC | QVQLVQSGAEVKKPGASVKVSC | 1.13 |
| | RASENVYSNLAWYQQKPGKAP | KASGYTFTNYVILWVRQAPGQG | |
| | KLLIYAASNLQSGVPSRFSGSG | LEWMGYINPYNDGTKYNEKFKG | |
| | SGTDFTFTISSLQPEDIATYYCQ | RVTMTRDTSTSTVYMELSSLRS | |
| | HFWYSPFWFGQGTKLEIK (SEQ | EDTAVYYCARLLKYRRFSYYAID | |
| | ID NO: 111) | YWGQGTTVTVSS (SEQ ID NO: | |
| 10-A5 | DIQMTQSPSSLSASVGDRVTITC | QVQLVQSGAEVKKPGASVKVSC | 1 |
| | RASETVYSNLAWYQQKPGKAP | KASGYTFTNYDIIWVRQAPGQG | |
| | KLLIYAASNLQSGVPSRFSGSG | LEWMGYINPYNDGTKYNEKFKG | |
| | SGTDFTFTISSLQPEDIATYYCQ | RVTMTRDTSTSTVYMELSSLRS | |
| | HFWGSPFTFGQGTKLEIK (SEQ | EDTAVYYCARLLKYRRFRYYAID | |
| | ID NO: 112) | YWGQGTTVTVSS (SEQ ID NO: | |
| | | 113) | |
| 10-A7 | DIQMTQSPSSLSASVGDRVTITC | QVQLVQSGAEVKKPGASVKVSC | 3 |
| | RASENVYSHLAWYQQKPGKAP | KASGYTFTNYDIIWVRQAPGQG | |
| | KLLIYAASNLQSGVPSRFSGSG | LEWMGYINPYNGRREYNEKFKG | |
| | SGTDFTFTISSLQPEDIATYYCQ | RVTMTRDTSTSTVYMELSSLRS | |
| | HFWYSPFTFGQGTKLEIK (SEQ | EDTAVYYCARLLKYRRFRYYAID | |
| | ID NO: 114) | YWGQGTTVTVSS (SEQ ID NO: | |
| | | 16) | |
| 10-B9 | DIQMTQSPSSLSASVGDRVTITC | QVQLVQSGAEVKKPGASVKVSC | 4 |
| | RASEPVYSNLAWYQQKPGKAP | KASGYTFTNYDIIWVRQAPGQG | |
| | KLLIYAASNLQSGVPSRFSGSG | LEWMGYINPYNDGRKENEKFKG | |
| | SGTDFTFTISSLQPEDIATYYCQ | RVTMTRDTSTSTVYMELSSLRS | |
| | HFWGSPFTFGQGTKLEIK (SEQ | EDTAVYYCARLLKYRRFRYYAID | |
| | ID NO: 115) | YWGQGTTVTVSS (SEQ ID NO: | |
| | | 116) | |
| 10-C7 | DIQMTQSPSSLSASVGDRVTITC | QVQLVQSGAEVKKPGASVKVSC | 4.1 |
| | RASESVYSNLAWYQQKPGKAP | KASGYTFTNYDIIWVRQAPGQG | |
| | KLLIYAASNLQSGVPSRFSGSG | LEWMGHINPYNGRREYNEKFKG | |
| | SGTDFTFTISSLQPEDIATYYCQ | RVTMTRDTSTSTVYMELSSLRS | |
| | HFVYSPFTFGQGTKLEIK (SEQ | EDTAVYYCARLLKYRRFRYYAID | |
| | ID NO: 117) | YWGQGTTVTVSS (SEQ ID NO: | |
| | | 118) | |
| 10-C10 | DIQMTQSPSSLSASVGDRVTITC | QVQLVQSGAEVKKPGASVKVSC | 4.3 |
| | RASENVYRNLAWYQQKPGKAP | KASGYTFTNYDIIWVRQAPGQG | |
| | KLLIYAASNLQSGVPSRFSGSG | LEWMGYINPYNGRREYNEKFKG | |
| | SGTDFTFTISSLQPEDIATYYCQ | RVTMTRDTSTSTVYMELSSLRS | |
| | HFVYSPFTFGQGTKLEIK (SEQ | EDTAVYYCARLLKYRRFRYYAID | |
| | ID NO: 119) | YWGQGTTVTVSS (SEQ ID NO: | |
| | | 16) | |
| 10-D11 | DIQMTQSPSSLSASVGDRVTITC | QVQLVQSGAEVKKPGASVKVSC | 4.2 |
| | RASENVYRNLAWYQQKPGKAP | KASGYTFTNYDIIWVRQAPGQG | |
| | KLLIYAASNLQSGVPSRFSGSG | LEWMGYINPYNGRREYNEKFKG | |
| | SGTDFTFTISSLQPEDIATYYCQ | RVTMTRDTSTSTVYMELSSLRS | |
| | HFWYSPFTFGQGTKLEIK (SEQ | EDTAVYYCARLLKYRRFRYYAID | |
| | ID NO: 120) | YWGQGTTVTVSS (SEQ ID NO: | |
| | | 16) | |
| 10-E10 | DIQMTQSPSSLSASVGDRVTITC | QVQLVQSGAEVKKPGASVKVSC | 2.7 |
| | RASENVYSNLAWYQQKPGKAP | KASGYTFTNYDIIWVRQAPGQG | |
| | KLLIYAASNLQSGVPSRFSGSG | LEWMGYINPYNDGRKENEKFKG | |
| | SGTDFTFTISSLQPEDIATYYCQ | RVTMTRDTSTSTVYMELSSLRS | |
| | HFWGSPFTFGQGTKLEIK (SEQ | EDTAVYYCARLLKYRRFRYYAID | |
| | ID NO: 7) | YWGQGTTVTVSS (SEQ ID NO: | |
| | | 116) | |
| 10-F7 | DIQMTQSPSSLSASVGDRVTITC | QVQLVQSGAEVKKPGASVKVSC | 4.9 |
| | RASENVYSHLAWYQQKPGKAP | KASGYTFTNYDIIWVRQAPGQG | |
| | KLLIYAASNLQSGVPSRFSGSG | LEWMGYINPYNGRREYNEKFKG | |
| | SGTDFTFTISSLQPEDIATYYCQ | RVTMTRDTSTSTVYMELSSLRS | |
| | HFVYSPFTFGQGTKLEIK (SEQ | EDTAVYYCARLLKYRRFRYYAID | |
| | ID NO: 121) | YWGQGTTVTVSS (SEQ ID NO: | |
| | | 16) | |
| 10-F10 | DIQMTQSPSSLSASVGDRVTITC | QVQLVQSGAEVKKPGASVKVSC | 4.1 |
| | RASENVYLNLAWYQQKPGKAP | KASGYTFTNYDIIWVRQAPGQG | |
| | KLLIYAASNLQSGVPSRFSGSG | LEWMGYINPYNDGRKENEKFKG | |
| | SGTDFTFTISSLQPEDIATYYCQ | RVTMTRDTSTSTVYMELSSLRS | |
| | HFWGSPFTFGQGTKLEIK (SEQ | EDTAVYYCARLLKYRRFRYYAID | |
| | ID NO: 122) | YWGQGTTVTVSS (SEQ ID NO: | |
| | | 116) | |
| 10-F12 | DIQMTQSPSSLSASVGDRVTITC | QVQLVQSGAEVKKPGASVKVSC | 5.7 |
| | RASENVYRNLAWYQQKPGKAP | KASGYTFTNYDIIWVRQAPGQG | |
| | KLLIYAASNLQSGVPSRFSGSG | LEWMGYINPYNGRREYNEKFKG | |
| | SGTDFTFTISSLQPEDIATYYCQ | RVTMTRDTSTSTVYMELSSLRS | |
| | HFWGSPFTFGQGTKLEIK (SEQ | EDTAVYYCARLLKYRRFRYYAID | |
| | ID NO: 123) | YWGQGTTVTVSS (SEQ ID NO: | |
| | | 16) | |
| 10-G2 | DIQMTQSPSSLSASVGDRVTITC | QVQLVQSGAEVKKPGASVKVSC | 5.2 |
| | RASEAVYSNLAWYQQKPGKAP | KASGYTFTNYDIIWVRQAPGQG | |
| | KLLIYAASNLQSGVPSRFSGSG | LEWMGYINPYNGRREYNEKFKG | |
| | SGTDFTFTISSLQPEDIATYYCQ | RVTMTRDTSTSTVYMELSSLRS | |
| | HFWGSPFTFGQGTKLEIK (SEQ | EDTAVYYCARLLKYRRFRYYAID | |
| | ID NO: 124) | YWGQGTTVTVSS (SEQ ID NO: | |
| | | 16) | |
| 10-G3 | DIQMTQSPSSLSASVGDRVTITC | QVQLVQSGAEVKKPGASVKVSC | 4.8 |
| | RASENVYRNLAWYQQKPGKAP | KASGYTFTNYDIIWVRQAPGQG | |
| | KLLIYIASNLQSGVPSRFSGSGS | LEWMGYINPYNERTEYNEKFKG | |
| | GTDFTFTISSLQPEDIATYYCQH | RVTMTRDTSTSTVYMELSSLRS | |
| | FVYSPFTFGQGTKLEIK (SEQ ID | EDTAVYYCARLLKYRRFRYYAID | |
| | NO: 125) | YWGQGTTVTVSS (SEQ ID NO: | |
| | | 126) | |
| 11-B9 | DIQMTQSPSSLSASVGDRVTITC | QVQLVQSGAEVKKPGASVKVSC | 4.9 |
| | RASTNVYRNLAWYQQKPGKAP | KASGYTFTNYDIIWVRQAPGQG | |
| | KLLIYAASNLQSGVPSRFSGSG | LEWMGYINPYNGRREYNEKFKG | |
| | SGTDFTFTISSLQPEDIATYYCQ | RVTMTRDTSTSTVYMELSSLRS | |
| | HFWGSPFTFGQGTKLEIK (SEQ | EDTAVYYCARLLKYRRFRYYAID | |
| | ID NO: 127) | YWGQGTTVTVSS (SEQ ID NO: | |
| | | 16) | |
| 11-B11 | DIQMTQSPSSLSASVGDRVTITC | QVQLVQSGAEVKKPGASVKVSC | 5.5 |
| | RASEPVYSNLAWYQQKPGKAP | KASGYTFTNYDIIWVRQAPGQG | |
| | KLLIYAASNLQSGVPSRFSGSG | LEWMGYINPYNGRREYNEKFKG | |
| | SGTDFTFTISSLQPEDIATYYCQ | RVTMTRDTSTSTVYMELSSLRS | |
| | HFWGSPFTFGQGTKLEIK (SEQ | EDTAVYYCARLLKYRRFRYYAID | |
| | ID NO: 115) | YWGQGTTVTVSS (SEQ ID NO: | |
| | | 16) | |
| 11-C6 | DIQMTQSPSSLSASVGDRVTITC | QVQLVQSGAEVKKPGASVKVSC | 10.8 |
| | RASSNVYSNLAWYQQKPGKAP | KASGYTFTNYDIIWVRQAPGQG | |
| | KLLIYAASNLQSGVPSRFSGSG | LEWMGYINPYNGRREYNEKFKG | |
| | SGTDYTFTISSLQPEDIATYYCQ | RVTMTRDTSTSTVYMELSSLRS | |
| | HFWGSPFTFGQGTKLEIK (SEQ | EDTAVYYCARLLKYRRFRYYAID | |
| | ID NO: 128) | YWGQGTTVTVSS (SEQ ID NO: | |
| | | 16) | |
| 11-C7 | DIQMTQSPSSLSASVGDRVTITC | QVQLVQSGAEVKKPGASVKVSC | 4.9 |
| | RASENVYSHLAWYQQKPGKAP | KASGYTFTNYDIIWVRQAPGQG | |
| | KLLIYAASNLQSGVPSRFSGSG | LEWMGYINPYNRRIEYNEKFKG | |
| | SGTDFTFTISSLQPEDIATYYCQ | RVTMTRDTSTSTVYMELSSLRS | |
| | HFWGSPFTFGQGTKLEIK (SEQ | EDTAVYYCARLLKYRRFRYYAID | |
| | ID NO: 129) | YWGQGTTVTVSS (SEQ ID NO: | |
| | | 130) | |
| 11-D7 | DIQMTQSPSSLSASVGDRVTITC | QVQLVQSGAEVKKPGASVKVSC | 4.1 |
| | RASENVYRNLAWYQQKPGKAP | KASGYTFTNYDIIWVRQAPGQG | |
| | KLLIYAASNLQSGVPSRFSGSG | LEWMGYINPYNGRREYNEKFKG | |
| | SGTDFTFTISSLQPEDIATYYCQ | RVTMTRDTSTSTVYMELSSLRS | |
| | HFWGSPFTFGQGTKLEIK (SEQ | EDTAVYYCARLLKYRRFRYYAID | |
| | ID NO: 123) | YWGQGTTVTVSS (SEQ ID NO: | |
| | | 16) | |
| 11-D8 | DIQMTQSPSSLSASVGDRVTITC | QVQLVQSGAEVKKPGASVKVSC | 2.5 |
| | RASTNVYSNLAWYQQKPGKAP | KASGYTFTNYDIIWVRQAPGQG | |
| | KLLIYAASNLQSGVPSRFSGSG | LEWMGYINPYNGRREYNEKFKG | |
| | SGTDFTFTISSLQPEDIATYYCQ | RVTMTRDTSTSTVYMELSSLRS | |
| | HFWGSPFTFGQGTKLEIK (SEQ | EDTAVYYCARLLKYRRFRYYAID | |
| | ID NO: 131) | YWGQGTTVTVSS (SEQ ID NO: | |
| | | 16) | |
| 11-F9 | DIQMTQSPSSLSASVGDRVTITC | QVQLVQSGAEVKKPGASVKVSC | 6.2 |
| | RASENVYRNLAWYQQKPGKAP | KASGYTFTNYDIIWVRQAPGQG | |
| | KLLIYAASNLQSGVPSRFSGSG | LEWMGYINPYNDGRKENEKFKG | |
| | SGTDFTFTISSLQPEDIATYYCQ | RVTMTRDTSTSTVYMELSSLRS | |
| | HFWYSPFTFGQGTKLEIK (SEQ | EDTAVYYCARLLKYRRFRYYAID | |
| | ID NO: 120) | YWGQGTTVTVSS (SEQ ID NO: | |
| | | 116) | |
| 11-G2 | DIQMTQSPSSLSASVGDRVTITC | QVQLVQSGAEVKKPGASVKVSC | 1.5 |
| | RASENVYSNLAWYQQKPGKAP | KASGYTFTNYDIIWVRQAPGQG | |
| | KLLIYAASNLQSGVPSRFSGSG | LEWMGYINPYNDGRKENEKFKG | |
| | SGTDFTFTISSLQPEDIATYYCQ | RVTMTRDTSTSTVYMELSSLRS | |
| | HFWDSPFTFGQGTKLEIK (SEQ | EDTAVYYCARLLKYRRFRYYAID | |
| | ID NO: 132) | YWGQGTTVTVSS (SEQ ID NO: | |
| | | 116) | |
| 11-G5 | DIQMTQSPSSLSASVGDRVTITC | QVQLVQSGAEVKKPGASVKVSC | 5.9 |
| | RASENVYYNLAWYQQKPGKAP | KASGYTFTNYDIIWVRQAPGQG | |
| | KLLIYAASNLQSGVPSRFSGSG | LEWMGYINPYNGRREYNEKFKG | |
| | SGTDFTFTISSLQPEDIATYYCQ | RVTMTRDTSTSTVYMELSSLRS | |
| | HFWYSPFTFGQGTKLEIK (SEQ | EDTAVYYCARLLKYRRFRYYAID | |
| | ID NO: 59) | YWGQGTTVTVSS (SEQ ID NO: | |
| | | 16) | |
| 11-G10 | DIQMTQSPSSLSASVGDRVTITC | QVQLVQSGAEVKKPGASVKVSC | 3.0 |
| | RASENVYSNLAWYQQKPGKAP | KASGYTFTNYDIIWVRQAPGQG | |
| | KLLIYAASNLQSGVPSRFSGSG | LEWMGYINPYNGRREYNEKFKG | |
| | SGTDFTFTISSLQPEDIATYYCQ | RVTMTRDTSTSTVYMELSSLRS | |
| | HFWGSPFTFGQGTKLEIK (SEQ | EDTAVYYCARLLKYRRFRYYAID | |
| | ID NO: 7) | YWGQGTTVTVSS (SEQ ID NO: | |
| | | 16) | |
| 12-A8 | DIQMTQSPSSLSASVGDRVTITC | QVQLVQSGAEVKKPGASVKVSC | 3.3 |
| | RASENVYSNLAWYQQKPGKAP | KASGYTFTNYDIIWVRQAPGQG | |
| | KLLIYAASNLQSGVPSRFSGSG | LEWMGYINPYNDGRKENEKFKG | |
| | SGTDFTFTISSLQPEDIATYYCQ | RVTMTRDTSTSTVYMELSSLRS | |
| | HFWGSPFTFGQGTKLEIK (SEQ | EDTAVYYCARLLKYRRFRYYAID | |
| | ID NO: 7) | YWGQGTTVTVSS (SEQ ID NO: | |
| | | 116) | |
| 12-B5 | DIQMTQSPSSLSASVGDRVTITC | QVQLVQSGAEVKKPGASVKVSC | 10 |
| | RASENVYSHLAWYQQKPGKAP | KASGYTFTNYDIIWVRQAPGQG | |
| | KLLIYAASNLQSGVPSRFSGSG | LEWMGYINPYNRRIEYNEKFKG | |
| | SGTDYTFTISSLQPEDIATYYCQ | RVTMTRDTSTSTVYMELSSLRS | |
| | HFWGSPFTFGQGTKLEIK (SEQ | EDTAVYYCARLLKYRRFRYYAID | |
| | ID NO: 89) | YWGQGTTVTVSS (SEQ ID NO: | |
| | | 130) | |
| 12-C8 | DIQMTQSPSSLSASVGDRVTITC | QVQLVQSGAEVKKPGASVKVSC | 8 |
| | RASKNVYSNLAWYQQKPGKAP | KASGYTFTNYDIIWVRQAPGQG | |
| | KLLIYAASNLQSGVPSRFSGSG | LEWMGYINPYNGRREYNEKFKG | |
| | SGTDFTFTISSLQPEDIATYYCQ | RVTMTRDTSTSTVYMELSSLRS | |
| | HFWGSPFTFGQGTKLEIK (SEQ | EDTAVYYCARLLKYRRFRYYAID | |
| | ID NO: 133) | YWGQGTTVTVSS (SEQ ID NO: | |
| | | 16) | |
| 12-C9 | DIQMTQSPSSLSASVGDRVTITC | QVQLVQSGAEVKKPGASVKVSC | 0.8 |
| | RASKNVYSNLAWYQQKPGKAP | KASGYTFTNYDIIWVRQAPGQG | |
| | KLLIYAASNLQSGVPSRFSGSG | LEWMGYINPYNGRTEYNEKFKG | |
| | SGTDFTFTISSLQPEDIATYYCQ | RVTMTRDTSTSTVYMELSSLRS | |
| | QFWYSPFTFGQGTKLEIK (SEQ | EDTAVYYCARLLKYRRFRYYAID | |
| | ID NO: 134) | YWGQGTTVTVSS (SEQ ID NO: | |
| | | 16) | |
| 12-D8 | DIQMTQSPSSLSASVGDRVTITC | QVQLVQSGAEVKKPGASVKVSC | 10 |
| | RASENVYSNTAWYQQKPGKAP | KASGYTFTNYDIIWVRQAPGQG | |
| | KLLIYAASNLQSGVPSRFSGSG | LEWMGYINPYNGRREYNEKFKG | |
| | SGTDYTFTISSLQPEDIATYYCQ | RVTMTRDTSTSTVYMELSSLRS | |
| | HFWGSPFTFGQGTKLEIK (SEQ | EDTAVYYCARLLKYRRFRYYAID | |
| | ID NO: 135) | YWGQGTTVTVSS (SEQ ID NO: | |
| | | 16) | |
| 12-E5 | DIQMTQSPSSLSASVGDRVTITC | QVQLVQSGAEVKKPGASVKVSC | 7.5 |
| | RASEPVYSNLAWYQQKPGKAP | KASGYTFTNYDIIWVRQAPGQG | |
| | KLLIYAASNLQSGVPSRFSGSG | LEWMGYINPYNGRREYNEKFKG | |
| | SGTDFTFTISSLQPEDIATYYCQ | RVTMTRDTSTSTVYMELSSLRS | |
| | HFWGSPFTFGQGTKLEIK (SEQ | EDTAVYYCARLLKYRRFRYYAID | |
| | ID NO: 115) | YWGQGTTVTVSS (SEQ ID NO: | |
| | | 16) | |
| 12-E9 | DIQMTQSPSSLSASVGDRVTITC | QVQLVQSGAEVKKPGASVKVSC | 3.5 |
| | RASESVYNNLAWYQQKPGKAP | KASGYTFTNYDIIWVRQAPGQG | |
| | KLLIYAASNLQSGVPSRFSGSG | LEWMGYINPYNDGTKYNEKFKY | |
| | SGTDFTFTISSLQPEDIATYYCQ | RVTMTRDTSTSTVYMELSSLRS | |
| | HFWGSPFTFGQGTKLEIK (SEQ | EDTAVYYCARLLKYRRFRYYAID | |
| | ID NO: 136) | YWGQGTTVTVSS (SEQ ID NO: | |
| | | 137) | |
| 12-E11 | DIQMTQSPSSLSASVGDRVTITC | QVQLVQSGAEVKKPGASVKVSC | 11 |
| | RASEPVYNNLAWYQQKPGKAP | KASGYTFTNYDIIWVRQAPGQG | |
| | KLLIYAASNLQSGVPSRFSGSG | LEWMGYINPYNGRREYNEKFKG | |
| | SGTDYTFTISSLQPEDIATYYCQ | RVTMTRDTSTSTVYMELSSLRS | |
| | HFWYSPFTFGQGTKLEIK (SEQ | EDTAVYYCARLLKYRRFRYYAID | |
| | ID NO: 138) | YWGQGTTVTVSS (SEQ ID NO: | |
| | | 16) | |
| 12-H6 | DIQMTQSPSSLSASVGDRVTITC | QVQLVQSGAEVKKPGASVKVSC | 12 |
| | RASENVYRNLAWYQQKPGKAP | KASGYTFTNYDIIWVRQAPGQG | |
| | KLLIYAASNLQSGVPSRFSGSG | LEWMGYINPYNRRREYNEKFKG | |
| | SGTDYTFTISSLQPEDIATYYCQ | RVTMTRDTSTSTVYMELSSLRS | |
| | HFWGSPFTFGQGTKLEIK (SEQ | EDTAVYYCARLLKYRRFRYYAID | |
| | ID NO: 104) | YWGQGTTVTVSS (SEQ ID NO: | |
| | | 8) | |
| 13-A12 | DIQMTQSPSSLSASVGDRVTITC | QVQLVQSGAEVKKPGASVKVSC | 13 |
| | RASENVYRNLAWYQQKPGKAP | KASGYTFTNYDIIWVRQAPGQG | |
| | KLLIYAASNLQSGVPSRFSGSG | LEWMGYINPYNDGRKENEKFKG | |
| | SGTDYTFTISSLQPEDIATYYCQ | RVTMTRDTSTSTVYMELSSLRS | |
| | HFWYSPFTF GQGTKLEIK (SEQ | EDTAVYYCARLLKYRRFRYYAID | |
| | ID NO: 85) | YWGQGTTVTVSS (SEQ ID NO: | |
| | | 116) | |
| 13-B2 | DIQMTQSPSSLSASVGDRVTITC | QVQLVQSGAEVKKPGASVKVSC | 13 |
| | RASKPVYSNLAWYQQKPGKAP | KASGYTFTNYDIIWVRQAPGQG | |
| | KLLIYAASNLQSGVPSRFSGSG | LEWMGYINPYNGRREYNEKFKG | |
| | SGTDYTFTISSLQPEDIATYYCQ | RVTMTRDTSTSTVYMELSSLRS | |
| | HFWGSPFTF GQGTKLEIK (SEQ | EDTAVYYCARLLKYRRFRYYAID | |
| | ID NO: 139) | YWGQGTTVTVSS (SEQ ID NO: | |
| | | 16) | |
| 13-B3 | DIQMTQSPSSLSASVGDRVTITC | QVQLVQSGAEVKKPGASVKVSC | 13 |
| | RASENVYNNLAWYQQKPGKAP | KASGYTFTNYDIIWVRQAPGQG | |
| | KLLIYAASNLQSGVPSRFSGSG | LEWMGYINPYNGRREYNEKFKG | |
| | SGTDYTFTISSLQPEDIATYYCQ | RVTMTRDTSTSTVYMELSSLRS | |
| | HFWGSPFTF GQGTKLEIK (SEQ | EDTAVYYCARLLKYRRFRYYAID | |
| | ID NO: 140) | YWGQGTTVTVSS (SEQ ID NO: | |
| | | 16) | |
| 13-B4 | DIQMTQSPSSLSASVGDRVTITC | QVQLVQSGAEVKKPGASVKVSC | 13 |
| | RASENVYMNLAWYQQKPGKAP | KASGYTFTNYDIIWVRQAPGQG | |
| | KLLIYAASNLQSGVPSRFSGSG | LEWMGYINPYNDGRKENEKFKG | |
| | SGTDYTFTISSLQPEDIATYYCQ | RVTMTRDTSTSTVYMELSSLRS | |
| | HFWYSPFTF GQGTKLEIK (SEQ | EDTAVYYCARLLKYRRFRYYAID | |
| | ID NO: 141) | YWGQGTTVTVSS (SEQ ID NO: | |
| | | 116) | |
| 13-C5 | DIQMTQSPSSLSASVGDRVTITC | QVQLVQSGAEVKKPGASVKVSC | 13 |
| | RASEPVYSNLAWYQQKPGKAP | KASGYTFTNYDIIWVRQAPGQG | |
| | KLLIYAASNLQSGVPSRFSGSG | LEWMGYINPYNGRREYNEKFKG | |
| | SGTDYTFTISSLQPEDIATYYCQ | RVTMTRDTSTSTVYMELSSLRS | |
| | HFWGSPFTF GQGTKLEIK (SEQ | EDTAVYYCARLLKYRRFRYYAID | |
| | ID NO: 80) | YWGQGTTVTVSS (SEQ ID NO: | |
| | | 16) | |
| 13-C6 | DIQMTQSPSSLSASVGDRVTITC | QVQLVQSGAEVKKPGASVKVSC | 4.8 |
| | RASEPVYSNVAWYQQKPGKAP | KASGYTFTNYDIIWVRQAPGQG | |
| | KLLIYAASNLQSGVPSRFSGSG | LEWMGYINPYNGRREYNEKFKG | |
| | SGTDFTFTISSLQPEDIATYYCQ | RVTMTRDTSTSTVYMELSSLRS | |
| | HFWGSPFTF GQGTKLEIK (SEQ | EDTAVYYCARLLKYRRFRYYAID | |
| | ID NO: 142) | YWGQGTTVTVSS (SEQ ID NO: | |
| | | 16) | |
| 13-C7 | DIQMTQSPSSLSASVGDRVTITC | QVQLVQSGAEVKKPGASVKVSC | 13 |
| | RASEPVYSNLAWYQQKPGKAP | KASGYTFTNYDIIWVRQAPGQG | |
| | KLLIYAASNLQSGVPSRFSGSG | LEWMGYINPYNGRREYNEKFKG | |
| | SGTDYTFTISSLQPEDIATYYCQ | RVTMTRDTSTSTVYMELSSLRS | |
| | HFWYSPFTF GQGTKLEIK (SEQ | EDTAVYYCARLLKYRRFRYYAID | |
| | ID NO: 143) | YWGQGTTVTVSS (SEQ ID NO: | |
| | | 16) | |
| 13-C11 | DIQMTQSPSSLSASVGDRVTITC | QVQLVQSGAEVKKPGASVKVSC | 13 |
| | RASENVYSHLAWYQQKPGKAP | KASGYTFTNYDIIWVRQAPGQG | |
| | KLLIYAASNLQSGVPSRFSGSG | LEWMGYINPYNGRREYNEKFKG | |
| | SGTDYTFTISSLQPEDIATYYCQ | RVTMTRDTSTSTVYMELSSLRS | |
| | HFWGSPFTF GQGTKLEIK (SEQ | EDTAVYYCARLLKYRRFRYYAID | |
| | ID NO: 89) | YWGQGTTVTVSS (SEQ ID NO: | |
| | | 16) | |
| 13-C12 | DIQMTQSPSSLSASVGDRVTITC | QVQLVQSGAEVKKPGASVKVSC | 13 |
| | RASENVYRNLAWYQQKPGKAP | KASGYTFTNYDIIWVRQAPGQG | |
| | KLLIYAASNLQSGVPSRFSGSG | LEWMGYINPYNGRREYNEKFKG | |
| | SGTDYTFTISSLQPEDIATYYCQ | RVTMTRDTSTSTVYMELSSLRS | |
| | HFVYSPFTF GQGTKLEIK (SEQ | EDTAVYYCARLLKYRRFRYYAID | |
| | ID NO: 157) | YWGQGTTVTVSS (SEQ ID NO: | |
| | | 16) | |
| 13-D5 | DIQMTQSPSSLSASVGDRVTITC | QVQLVQSGAEVKKPGASVKVSC | 13 |
| | RASENVTSNLAWYQQKPGKAP | KASGYTFTNYDIIWVRQAPGQG | |
| | KLLIYAASNLQSGVPSRFSGSG | LEWMGYINPYNGRREYNEKFKG | |
| | SGTDYTFTISSLQPEDIATYYCQ | RVTMTRDTSTSTVYMELSSLRS | |
| | HFWGSPFTF GQGTKLEIK (SEQ | EDTAVYYCARLLKYRRFRYYAID | |
| | ID NO: 158) | YWGQGTTVTVSS (SEQ ID NO: | |
| | | 16) | |
| 13-F12 | DIQMTQSPSSLSASVGDRVTITC | QVQLVQSGAEVKKPGASVKVSC | 13 |
| | RASENVYRNLAWYQQKPGKAP | KASGYTFTNYDIIWVRQAPGQG | |
| | KLLIYAASNLQSGVPSRFSGSG | LEWMGYINPYNGRREYNEKFKG | |
| | SGTDYTFTISSLQPEDIATYYCQ | RVTMTRDTSTSTVYMELSSLRS | |
| | HFWGSPFTF GQGTKLEIK (SEQ | EDTAVYYCARLLKYRRFRYYAID | |
| | ID NO: 104) | YWGQGTTVTVSS (SEQ ID NO: | |
| | | 16) | |
| 13-H3 | DIQMTQSPSSLSASVGDRVTITC | QVQLVQSGAEVKKPGASVKVSC | 13 |
| | RASENVYSNLAWYQQKPGKAP | KASGYTFTNYDIIWVRQAPGQG | |
| | KLLIYAASNLQSGVPSRFSGSG | LEWMGYINPYNDGRKENEKFKG | |
| | SGTDYTFTISSLQPEDIATYYCQ | RVTMTRDTSTSTVYMELSSLRS | |
| | HFWGSPFTF GQGTKLEIK (SEQ | EDTAVYYCARLLKYRRFRYYAID | |
| | ID NO: 14) | YWGQGTTVTVSS (SEQ ID NO: | |
| | | 116) | |
| 13-H6 | DIQMTQSPSSLSASVGDRVTITC | QVQLVQSGAEVKKPGASVKVSC | 13 |
| | RASENVYSHLAWYQQKPGKAP | KASGYTFTNYDIIWVRQAPGQG | |
| | KLLIYAASNLQSGVPSRFSGSG | LEWMGYINPYNDGRKENEKFKG | |
| | SGTDYTFTISSLQPEDIATYYCQ | RVTMTRDTSTSTVYMELSSLRS | |
| | HFVYSPFTF GQGTKLEIK (SEQ | EDTAVYYCARLLKYRRFRYYAID | |
| | ID NO: 146) | YWGQGTTVTVSS (SEQ ID NO: | |
| | | 116) | |
| 14-A6 | DIQMTQSPSSLSASVGDRVTITC | QVQLVQSGAEVKKPGASVKVSC | 2.8 |
| | HASENVYSNLAWYQQKPGKAP | KASGYTFTNYDIIWVRQAPGQG | |
| | KLLIYAASNLQSGVPSRFSGSG | LEWMGYINPYNRRREYNEKFKG | |
| | SGTDFTFTISSLQPEDIATYYCQ | RVTMTRDTSTSTVYMELSSLRS | |
| | HFWGSPFTFGQGTKLEIK | EDTAVYYCARLLKYRRFRYYAID | |
| | (SEQ ID NO: 145) | YWGQGTTVTVSS (SEQ ID NO: | |
| | | 8) | |
| 14-B1 | DIQMTQSPSSLSASVGDRVTITC | QVQLVQSGAEVKKPGASVKVSC | 13 |
| | RASEPVYSNLAWYQQKPGKAP | KASGYTFTNYDIIWVRQAPGQG | |
| | KLLIYAASNLQSGVPSRFSGSG | LEWMGYINPYNDGRKENEKFKG | |
| | SGTDYTFTISSLQPEDIATYYCQ | RVTMTRDTSTSTVYMELSSLRS | |
| | HFWGSPFTFGQGTKLEIK (SEQ | EDTAVYYCARLLKYRRFRYYAID | |
| | ID NO: 80) | YWGQGTTVTVSS (SEQ ID NO: | |
| | | 116) | |
| 15-H1 | DIQMTQSPSSLSASVGDRVTITC | QVQLVQSGAEVKKPGASVKVSC | 2.1 |
| | RASENVYSNLAWYQQKPGKAP | KASGYTFTNYDIIWVRQAPGQG | |
| | KLLIYAASNLQSGVPSRFSGSG | LEWMGYINPYNDGTKYNEKFKG | |
| | SGTDFTFTISSLQPEDIATYYCQ | RVTMTRDTSTSTVYMELSSLRS | |
| | HFWESPFTFGQGTKLEIK (SEQ | EDTAVYYCARLLKYACFREEAID | |
| | ID NO: 147) | YWGQGTTVTVSS (SEQ ID NO: | |
| | | 148) | |
| 15-G6 | DIQMTQSPSSLSASVGDRVTITC | QVQLVQSGAEVKKPGASVKVSC | 1.8 |
| | RTSENVYSNLAWYQQKPGKAP | KASGYTFTNYDIIWVRQAPGQG | |
| | KLLIYAASNLQSGVPSRFSGSG | LEWMGYINPYNDGTKYNEKFKG | |
| | SGTDFTFTISSLQPEDIATYYCQ | RVTMTRDTSTSTVYMELSSLRS | |
| | QFWYSPFTFGQGTKLEIK (SEQ | EDTAVYYCARLLKYRRFKYYAID | |
| | ID NO: 149) | YWGQGTTVTVSS (SEQ ID NO: | |
| | | 150) | |
| 16-E4 | DIQMTQSPSSLSASVGDRVTITC | QVQLVQSGAEVKKPGASVKVSC | 7 |
| | RASENVYSNLAWYQQKPGKAP | KASGYTFTNYDIIWVRQAPGQG | |
| | KLLIYAASNLQSGVPSRFSGSG | LEWMGYINPYNDGTKYNEKFKG | |
| | SGTDFTFTISSLQPEDIATYYCQ | RVTMTRDTSTSTVYMELSSLRS | |
| | HFWYSPWTFGQGTKLEIK (SEQ | EDTAVYYCARLLKYRRFRYYAID | |
| | ID NO: 100) | YWGQGTTVTVSS (SEQ ID NO: | |
| | | 12) | |
| 16-C1 | DIQMTQSPSSLSASVGDRVTITC | QVQLVQSGAEVKKPGASVKVSC | 6 |
| | RASENVYSNLAWYQQKPGKAP | KASGYTFTNYDIIWVRQAPGQG | |
| | KLLIYAASNLQSGVPSRFSGSG | LEWMGYINPYNDGTKYNEKFKG | |
| | SGTDFTFTISSLQPEDIATYYCQ | RVTMTRDTSTSTVYMELSSLRS | |
| | HFWYSPWTFGQGTKLEIK (SEQ | EDTAVYYCARLLKYRRFKYYAID | |
| | ID NO: 100) | YWGQGTTVTVSS (SEQ ID NO: | |
| | | 150) | |
| 16-B2 | DIQMTQSPSSLSASVGDRVTITC | QVQLVQSGAEVKKPGASVKVSC | 5 |
| | RASENVYSNLAWYQQKPGKAP | KASGYTFTNYDIIWVRQAPGQG | |
| | KLLIYAASNLQSGVPSRFSGSG | LEWMGYINPYNDGTKYNEKFKG | |
| | SGTDFTFTISSLQPEDIATYYCQ | RVTMTRDTSTSTVYMELSSLRS | |
| | HFWGSPFTFGQGTKLEIK | EDTAVYYCARLLKYRRFRYYAID | |
| | (SEQ ID NO: 7) | YWGQGTTVTVSS (SEQ ID NO: | |
| | | 12) | |
| 16-B7 | DIQMTQSPSSLSASVGDRVTITC | QVQLVQSGAEVKKPGASVKVSC | 5 |
| | RASENVYSNLAWYQQKPGKAP | KASGYTFTNYDIIWVRQAPGQG | |
| | KLLIYAASNLADGVPSRFSGSG | LEWMGYINPYNDGTKYNEKFKG | |
| | SGTDFTFTISSLQPEDIATYYCQ | RVTMTRDTSTSTVYMELSSLRS | |
| | HFWASPFTFGQGTKLEIK | EDTAVYYCARLLKYRRFRYYAID | |
| | (SEQ ID NO: 151) | YWGQGTTVTVSS (SEQ ID NO: | |
| | | 12) | |
| 16-H4 | DIQMTQSPSSLSASVGDRVTITC | QVQLVQSGAEVKKPGASVKVSC | 6 |
| | RTSESVYSNLAWYQQKPGKAP | KASGYTFTNYDIIWVRQAPGQG | |
| | KLLIYAASNLQSGVPSRFSGSG | LEWMGYINPYNDGTKYNEKFKG | |
| | SGTDFTFTISSLQPEDIATYYCQ | RVTMTRDTSTSTVYMELSSLRS | |
| | HFWASPFTFGQGTKLEIK | EDTAVYYCARLLKYRRFRYYAID | |
| | (SEQ ID NO: 152) | YWGQGTTVTVSS (SEQ ID NO: | |
| | | 12) | |
| 16-A10 | DIQMTQSPSSLSASVGDRVTITC | QVQLVQSGAEVKKPGASVKVSC | 6 |
| | RTSENVYSNLAWYQQKPGKAP | KASGYTFTNYDIIWVRQAPGQG | |
| | KLLIYAASNLQSGVPSRFSGSG | LEWMGYINPYNDGTKYNEKFKG | |
| | SGTDFTFTISSLQPEDIATYYCQ | RVTMTRDTSTSTVYMELSSLRS | |
| | HFWYSPWTFGQGTKLEIK (SEQ | EDTAVYYCARLLKYRRFRYYAID | |
| | ID NO: 153) | YWGQGTTVTVSS (SEQ ID NO: | |
| | | 12) | |
| 16-F4 | DIQMTQSPSSLSASVGDRVTITC | QVQLVQSGAEVKKPGASVKVSC | 5 |
| | RASENVYSNLAWYQQKPGKAP | KASGYTFTNYDIIWVRQAPGQG | |
| | KLLIYAASNLADGVPSRFSGSG | LEWMGYINPYNDGTKYNEKFKG | |
| | SGTDFTFTISSLQPEDIATYYCQ | RVTMTRDTSTSTVYMELSSLRS | |
| | HFWTSPFTFGQGTKLEIK | EDTAVYYCARLLKYRRFRYYAID | |
| | (SEQ ID NO: 154) | YWGQGTTVTVSS (SEQ ID NO: | |
| | | 12) | |
| 16-G9 | DIQMTQSPSSLSASVGDRVTITC | QVQLVQSGAEVKKPGASVKVSC | 4 |
| | RASENVYSNLAWYQQKPGKAP | KASGYTFTNYDIIWVRQAPGQG | |
| | KLLIYAASNLQSGVPSRFSGSG | LEWMGYINPYNDGTKYNEKFKG | |
| | SGTDFTFTISSLQPEDIATYYCQ | RVTMTRDTSTSTVYMELSSLRS | |
| | HFWYSPFGFGQGTKLEIK (SEQ | EDTAVYYCARLLKYRRFRYYAID | |
| | ID NO: 70) | YWGQGTTVTVSS (SEQ ID NO: | |
| | | 12) | |
| 16-D6 | DIQMTQSPSSLSASVGDRVTITC | QVQLVQSGAEVKKPGASVKVSC | 1 |
| | RASENVYSNLAWYQQKPGKAP | KASGYTFTNYDIIWVRQAPGQG | |
| | KLLIYAASNLADGVPSRFSGSG | LEWMGYINPYNDGTKYNEKFKG | |
| | SGTDFTFTISSLQPEDIATYYCQ | RVTMTRDTSTSTVYMELSSLRS | |
| | DFWYSPFTFGQGTKLEIK (SEQ | EDTAVYYCARLLKYRRFKYYAID | |
| | ID NO: 155) | YWGQGTTVTVSS (SEQ ID NO: | |
| | | 150) | |
| 16-A6 | DIQMTQSPSSLSASVGDRVTITC | QVQLVQSGAEVKKPGASVKVSC | 1 |
| | RTSENVYSNLAWYQQKPGKAP | KASGYTFTNYDIIWVRQAPGQG | |
| | KLLIYAASNLADGVPSRFSGSG | LEWMGYINPYNDGTKYNEKFKG | |
| | SGTDFTFTISSLQPEDIATYYCQ | RVTMTRDTSTSTVYMELSSLRS | |
| | HFWASPFTFGQGTKLEIK | EDTAVYYCARLLKYRRFRYYAID | |
| | (SEQ ID NO: 156) | YWGQGTTVTVSS (SEQ ID NO: | |
| | | 12) | |
| 16-C3 | DIQMTQSPSSLSASVGDRVTITC | QVQLVQSGAEVKKPGASVKVSC | 1 |
| | RASENVYSNLAWYQQKPGKAP | KASGYTFTNYDIIWVRQAPGQG | |
| | KLLIYAASNLQSGVPSRFSGSG | LEWMGYINPYNDGTKYNEKFKG | |
| | SGTDFTFTISSLQPEDIATYYCQ | RVTMTRDTSTSTVYMELSSLRS | |
| | QFWYSPFTFGQGTKLEIK (SEQ | EDTAVYYCARLLKYRRFRYYAID | |
| | ID NO: 86) | YWGQGTTVTVSS (SEQ ID NO: | |
| | | 12) | |
| consensus A | DIQMTQSPSSLSASVGDRVTITC | QVQLVQSGAEVKKPGASVKVSC | |
| | X¹X²SX³X⁴VX⁵X⁶X⁷X⁸AWYQQKP | KASGYTFTNYX¹IX²WVRQAPGQ | |
| | GKAPKLLIYX⁹ASNLX¹⁰X¹¹GVPS | GLEWMGX³IX⁴X⁵X⁶X⁷X⁸X⁹X¹⁰X¹¹ | |
| | RFSGSGSGTDX¹²TFTISSLQPE | X¹²X¹³X¹⁴KFKX¹⁵RVTMTRDTSTS | |
| | DIATYYCQX¹³FX¹⁴X¹⁵X¹⁶X¹⁷X¹⁸ | TVYMELSSLRSEDTAVYYCARL | |
| | X¹⁹FGQGTKLEIK (SEQ ID NO: | LX¹⁶X¹⁷X¹⁸X¹⁹X²⁰X²¹X²²X²³AX²⁴ | |
| | 20), wherein | X²⁵X²⁶WGQGTTVTVSS (SEQ ID | |
| | X¹ is R or H; | NO: 21), wherein | |
| | X² is A or T; | X¹ is D or V; | |
| | X³ is E, T, S or K; | X² is I or L; | |
| | X⁴ is N, P, T, S or A; | X³ is Y or H; | |
| | X⁵ is Y or T; | X⁴ is N, S or A; | |
| | X⁶ is S, R, L, Y, N or M; | X⁵ is P or A; | |
| | X⁷ is N or H; | X⁶ is Y or A; | |
| | X⁸ is L, V or T; | X⁷ is N, Q, V or A; | |
| | X⁹ is A or I; | X⁸ is G, R, D, A or E; | |
| | X¹⁰ is Q or A; | X⁹ is R or G; | |
| | X¹¹ is S or D; | X¹⁰ is R, T, K or I; | |
| | X¹² is f or Y; | X¹¹ is E or K; | |
| | X¹³ is H, G, N, V, D or Q; | X¹² is Y, E or A; | |
| | X¹⁴ is W, Y, D, S, K, G or V; | X¹³ is N or A; | |
| | X¹⁵ is Y, K, V, W, G, Q, A, L, H, | X¹⁴ is E or A; | |
| | M, E, T or D; | X¹⁵ is G or Y; | |
| | X¹⁶ is S, R or L; | X¹⁶ is K, A or R; | |
| | X¹⁷ is P, G or W; | X¹⁷ is Y or A; | |
| | X¹⁸ is F, C or W; and | X¹⁸ is R or A; | |
| | X¹⁹ is T, G, I, K,V, L, A or W | X¹⁹ is R, C, A or P; | |
| | | X²⁰ is F, E, A, H, m or L; | |
| | | X²¹ is R, S, A, K, T or Q; | |
| | | X²² is Y, E, A or F; | |
| | | X²³ is Y, E or A; | |
| | | X²⁴ is I, E or A; | |
| | | X²⁵ is D or H; and | |
| | | X²⁶ is Y, E or V | |
| consensus B | DIQMTQSPSSLSASVGDRVTITC | QVQLVQSGAEVKKPGASVKVSC | |
| | X¹X²SEX³VYSNX⁴AWYQQKPGK | KASGYTFTNYX¹IX²WVRQAPGQ | |
| | APKLLIYAASNLX⁵X⁶GVPSRFSG | GLEWMGYINPYNX³X⁴X⁵X⁶YNEK | |
| | SGSGTDX⁷TFTISSLQPEDIATYY | FKGRVTMTRDTSTSTVYMELSS | |
| | CQHFWX⁸SPFTFGQGTKLEIK | LRSEDTAVYYCARLLKYRRFX⁷Y | |
| | (SEQ ID NO: 170), wherein | YAIDYWGQGTTVTVSS (SEQ ID | |
| | X¹ is R or H; | NO: 171), wherein | |
| | X² is A or T; | X¹ is D or V; | |
| | X³ is N or P; | X² is I or L; | |
| | X⁴ is L or V; | X³ is G; R or D; | |
| | X⁵ is Q or A; | X⁴ is R or G; | |
| | X⁶ is S or D; | X⁵ is R or T; | |
| | X⁷ is F or Y; and | X⁶ is E or K; and | |
| | X⁸ is Y or G | X⁷ is R or S | |

*K_{off} measured at 37° C.

In Table 2, Chothia CDR regions are shown in bold, and Kabat CDR regions are underlined. The T_{1/2} for each antibody for TrkB is shown in minutes. The T_{1/2} was calculated by Log 2 divided by k_{off} (T_{1/2}=Log 2/K_{off}). K_{off} of each antibody (measured as Fab) was measured at 25° C. unless otherwise indicated.

The invention also provides CDR portions of antibodies to TrkB (including Chothia and Kabat CDRs). Determination of CDR regions is well within the skill of the art. It is understood that in some embodiments, CDRs can be a combination of the Kabat and Chothia CDR (also termed “combined CDRs” or “extended CDRs”). In some embodiments, the CDRs are the Kabat CDRs. In other embodiments, the CDRs are the Chothia CDRs. In other words, in embodiments with more than one CDR, the CDRs may be any of Kabat, Chothia, combination CDRs, or combinations thereof.

The binding affinity (K_{D}) of a TrkB agonist antibody to TrkB can be about 0.002 to about 200 nM. In some embodiments, the binding affinity is any of about 200 nM, 100 nM, about 50 nM, about 10 nM, about 1 nM, about 500 pM, about 100 pM, about 60 pM, about 50 pM, about 20 pM, about 15 pM, about 10 pM, about 5 pM, or about 2 pM. In some embodiments, the binding affinity is less than any of about 250 nM, about 200 nM, about 100 nM, about 50 nM, about 10 nM, about 1 nM, about 500 pM, about 100 pM, or about 50 pM.

The invention also provides methods of making any of these antibodies or polypeptides. The antibodies of this invention can be made by procedures known in the art. The polypeptides can be produced by proteolytic or other degradation of the antibodies, by recombinant methods (i.e., single or fusion polypeptides) as described above or by chemical synthesis. Polypeptides of the antibodies, especially shorter polypeptides up to about 50 amino acids, are conveniently made by chemical synthesis. Methods of chemical synthesis are known in the art and are commercially available. For example, an antibody could be produced by an automated polypeptide synthesizer employing the solid phase method. See also, U.S. Pat. Nos. 5,807,715; 4,816,567; and 6,331,415.

In another alternative, the antibodies can be made recombinantly using procedures that are well known in the art. In some embodiments, a polynucleotide comprises a sequence encoding the heavy chain and/or the light chain variable regions of antibody C2. See, Table 2. In other embodiments, a polynucleotide comprises a sequence encoding the heavy chain and/or the light chain variable regions of antibody RN1026A. The sequence encoding the antibody of interest may be maintained in a vector in a host cell and the host cell can then be expanded and frozen for future use. Vectors (including expression vectors) and host cells are further described herein.

The invention also encompasses scFv of antibodies of this invention. Single chain variable region fragments are made by linking light and/or heavy chain variable regions by using a short linking peptide. Bird et al. (1988) Science 242:423-426. An example of a linking peptide is (GGGGS)₃ (SEQ ID NO: 172), which bridges approximately 3.5 nm between the carboxy terminus of one variable region and the amino terminus of the other variable region. Linkers of other sequences have been designed and used. Bird et al. (1988), supra. Linkers should be short, flexible polypeptides and preferably comprised of less than about 20 amino acid residues. Linkers can in turn be modified for additional functions, such as attachment of drugs or attachment to solid supports. The single chain variants can be produced either recombinantly or synthetically. For synthetic production of scFv, an automated synthesizer can be used. For recombinant production of scFv, a suitable plasmid containing polynucleotide that encodes the scFv can be introduced into a suitable host cell, either eukaryotic, such as yeast, plant, insect or mammalian cells, or prokaryotic, such as E. coli. Polynucleotides encoding the scFv of interest can be made by routine manipulations such as ligation of polynucleotides. The resultant scFv can be isolated using standard protein purification techniques known in the art.

Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al., 1993, Proc. Natl. Acad. Sci. USA 90:6444-6448; Poljak, R. J., et al., 1994, Structure 2:1121-1123).

For example, bispecific antibodies, monoclonal antibodies that have binding specificities for at least two different antigens, can be prepared using the antibodies disclosed herein. Methods for making bispecific antibodies are known in the art (see, e.g., Suresh et al., 1986, Methods in Enzymology 121:210). Traditionally, the recombinant production of bispecific antibodies was based on the coexpression of two immunoglobulin heavy chain-light chain pairs, with the two heavy chains having different specificities (Millstein and Cuello, 1983, Nature 305, 537-539).

According to one approach to making bispecific antibodies, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2 and CH3 regions. It is preferred to have the first heavy chain constant region (CH1), containing the site necessary for light chain binding, present in at least one of the fusions. DNAs encoding the immunoglobulin heavy chain fusions and, if desired, the immunoglobulin light chain, are inserted into separate expression vectors, and are cotransfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In one approach, the bispecific antibodies are composed of a hybrid immunoglobulin heavy chain with a first binding specificity in one arm, and a hybrid immunoglobulin heavy chain-light chain pair (providing a second binding specificity) in the other arm. This asymmetric structure, with an immunoglobulin light chain in only one half of the bispecific molecule, facilitates the separation of the desired bispecific compound from unwanted immunoglobulin chain combinations. This approach is described in PCT Publication No. WO 94/04690.

Heteroconjugate antibodies, comprising two covalently joined antibodies, are also within the scope of the invention. Such antibodies have been used to target immune system cells to unwanted cells (U.S. Pat. No. 4,676,980), and for treatment of HIV infection (PCT Publication Nos. WO 91/00360 and WO 92/200373; EP 03089). Heteroconjugate antibodies may be made using any convenient cross-linking methods. Suitable cross-linking agents and techniques are well known in the art, and are described in U.S. Pat. No. 4,676,980.

Chimeric or hybrid antibodies also may be prepared in vitro using known methods of synthetic protein chemistry, including those involving cross-linking agents. For example, immunotoxins may be constructed using a disulfide exchange reaction or by forming a thioether bond. Examples of suitable reagents for this purpose include iminothiolate and methyl-4-mercaptobutyrimidate.

Humanized antibodies comprising one or more CDRs of any antibody of Table 2, one or more CDRs derived from any antibody of Table 2 or CDR of Table 3, can be made using any methods know in the art. For example, four general steps may be used to humanize a monoclonal antibody. These are: (1) determining the nucleotide and predicted amino acid sequence of the starting antibody light and heavy variable domains (2) designing the humanized antibody, i.e., deciding which antibody framework region to use during the humanizing process (3) the actual humanizing methodologies/techniques and (4) the transfection and expression of the humanized antibody. See, for example, U.S. Pat. Nos. 4,816,567; 5,807,715; 5,866,692; 6,331,415; 5,530,101; 5,693,761; 5,693,762; 5,585,089; and 6,180,370.

TABLE 3
Heavy Chain

| mAb | CDRH1 | CDRH2 | CDRH3 |
|---|---|---|---|
| RN1026A | GYTFTNYDII (SEQ ID | YINPYNRRREYNEKF | LLKYRRFRYYAIDY |
| | NO: 1) | KG (SEQ ID NO: 2) | (SEQ ID NO: 3) |
| T1-HuC1 | NYDII (SEQ ID NO: 24) | PYNDGT (SEQ ID NO: | LLKYRRFSYYAIDY |
| | | 25) | (SEQ ID NO: 26) |
| C2 | NYDII (SEQ ID NO: 24) | PYNDGT (SEQ ID NO: | LLKYRRFRYYAIDY |
| | | 25) | (SEQ ID NO: 3) |
| A2 | NYDII (SEQ ID NO: 24) | PYNDGT (SEQ ID NO: | LLKYRRFRYYAIDY |
| | | 25) | (SEQ ID NO: 3) |
| 4A6 | NYDII (SEQ ID NO: 24) | PYNRRR (SEQ ID NO: | LLKYRRFRYYAIDY |
| | | 27) | (SEQ ID NO: 3) |
| 4B12 | NYDII (SEQ ID NO: 24) | PYNGRR (SEQ ID NO: | LLKYRRFRYYAIDY |
| | | 28) | (SEQ ID NO: 3) |
| Heavy | GYTFTNYX¹IX² (SEQ | X¹IX²X³X⁴X⁵X⁶X⁷X⁸X⁹X¹⁰ | LLX¹X²X³X⁴X⁵X⁶X⁷X⁸ |
| chain | ID NO: 159), wherein | X¹¹X¹²KFKX¹³ (SEQ | AX⁹X¹⁰X¹¹ (SEQ ID |
| CDR | X¹ is D or V; and X² is I | ID NO: 165), wherein | NO: 166), wherein |
| consensus A | or L | X¹ is Y or H; X² is N; S | X¹ is K, A or R; X² is |
| | | or A; X³ is P or A; X⁴ is | Y or A; X³ is R or A; |
| | | Y or A; X⁵ is N, Q, V or | X⁴ is R, C, A or P; |
| | | A; X⁶ is G, R, D, A or E; | X⁵ is F, E, A, H, M or |
| | | X⁷ is R or G; X⁸ is R, T, | L; X⁶ is R, S, A, K, T |
| | | K or I; X⁹ is E or K; X¹⁰ | or Q; X⁷ is Y, E, A or |
| | | is Y, E or A; X¹¹ is N or | F; X⁸ is Y, E or A; X⁹ |
| | | A; X¹² is E or A; and | is I, E or A; X¹⁰ is D |
| | | X¹³ is G or Y | or H, and X¹¹ is Y, E |
| | | | or V |
| Heavy | GYTFTNYX¹IX² (SEQ | YINPYNX¹X²X³X⁴YNE | LLKYRRFXYYAIDY |
| chain | ID NO: 159), wherein | KFKG (SEQ ID NO: | (SEQ ID NO: 161), |
| CDR | X¹ is D or V; and X² is I | 160), wherein X¹ is G, | wherein X is R or S |
| consensus B | or L | R or D; X² is R or G; X³ | |
| | | is R or T; and X⁴ is E or K | |

Light Chain

| mAb | CDRL1 | CDRL2 | CDRL3 |
|---|---|---|---|
| RN1026A | RASENVYSNLA | AASNLQS (SEQ ID | QHFWGSPFT (SEQ |
| | (SEQ ID NO: 4) | NO: 5) | ID NO: 6) |
| T1-HuC1 | RASENVYSNLA | AASNLAD (SEQ ID | QHFWYSPFT (SEQ |
| | (SEQ ID NO: 4) | NO: 17) | ID NO: 18) |
| C2 | RTSENVYSNLA | AASNLQS (SEQ ID | QHFWGSPFT (SEQ |
| | (SEQ ID NO: 19) | NO: 5) | ID NO: 6) |
| A2 | RASENVYSNLA | AASNLQS (SEQ ID | QHFWYSPWT |
| | (SEQ ID NO: 4) | NO: 5) | (SEQ ID NO: 177) |
| 4A6 | HASENVYSNLA | AASNLQS (SEQ ID | QHFWGSPFT (SEQ |
| | (SEQ ID NO: 22) | NO: 5) | ID NO: 6) |
| 4B12 | RASEPVYSNVA | AASNLQS (SEQ ID | QHFWGSPFT (SEQ |
| | (SEQ ID NO: 23) | NO: 5) | ID NO: 6) |
| Light | X¹X²SX³X⁴VX⁵X⁶X⁷X⁸A | X¹ASNLX²X³ (SEQ ID | QX¹FX²X³X⁴X⁵X⁶X⁷ |
| chain | (SEQ ID NO: 167), | NO: 168), wherein X¹ is | (SEQ ID NO: 169), |
| CDR | wherein X¹ is R or H; | A or I; X² is Q or A; and | wherein X¹ is H, G, |
| consensus A | X² is A or T; X³ is E, T, | X³ is S or D | N, V, D or Q; X² is |
| | S or K; X⁴ is N, P, T, S | | W, Y, D, S, K, G or |
| | or A; X⁵ is Y or T; X⁶ is | | V; X³ is Y, K, V, W, |
| | S, R, L, Y, N or M; X⁷ is | | G, Q, A, L, H, M, E, |
| | N or H; and X⁸ is L, V | | T or D; X⁴ is S, R or |
| | or T | | L; X⁵ is P, G or W; |
| | | | X⁶ is F, C or W; and |
| | | | X⁷ is T, G, I, K, V, L, |
| | | | A or W |
| Light | X¹X²SEX³VYSNX⁴A | AASNLX¹X² (SEQ ID | QHFWXSPFT (SEQ |
| chain | (SEQ ID NO: 162), | NO: 163), wherein X¹ is | ID NO: 164), |
| CDR | wherein X¹ is R or H; | Q or A; and X² is S or D | wherein X is Y or G |
| consensus B | X² is A or T; X³ is N or | | |
| | P; and X⁴ is L or V | | |

In the recombinant humanized antibodies, the Fcγ portion can be modified to avoid interaction with Fcγ receptor and the complement and immune systems. The techniques for preparation of such antibodies are described in WO 99/58572. For example, the constant region may be engineered to more resemble human constant regions to avoid immune response if the antibody is used in clinical trials and treatments in humans. See, for example, U.S. Pat. Nos. 5,997,867 and 5,866,692.

Humanized antibody comprising the light or heavy chain variable regions of an antibody or its variants shown in Table 2, or one or more CDRs derived from the antibody or its variants shown in Table 3 can be made using any methods known in the art. In some embodiments, a light chain variable region framework can have an F to Y mutation at position 71 (F71Y).

Humanized antibodies may be made by any method known in the art.

The invention encompasses modifications to the antibodies shown in Tables 2 and 3, including functionally equivalent antibodies which do not significantly affect their properties and variants which have enhanced or decreased activity and/or affinity. For example, the amino acid sequence may be mutated to obtain an antibody with the desired binding affinity to TrkB. Modification of polypeptides is routine practice in the art and need not be described in detail herein. Examples of modified polypeptides include polypeptides with conservative substitutions of amino acid residues, one or more deletions or additions of amino acids which do not significantly deleteriously change the functional activity, or which mature (enhance) the affinity of the polypeptide for its ligand, or use of chemical analogs.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue or the antibody fused to an epitope tag. Other insertional variants of the antibody molecule include the fusion to the N- or C-terminus of the antibody of an enzyme or a polypeptide which increases the half-life of the antibody in the blood circulation.

Substitution variants have at least one amino acid residue in the antibody molecule removed and a different residue inserted in its place. The sites of greatest interest for substitutional mutagenesis include the hypervariable regions, but FR alterations are also contemplated. Conservative substitutions are shown in Table 4 under the heading of “conservative substitutions.” If such substitutions result in a change in biological activity, then more substantial changes, denominated “exemplary substitutions” in Table 4, or as further described below in reference to amino acid classes, may be introduced and the products screened.

TABLE 4
Amino Acid Substitutions

| | Conservative | |
|---|---|---|
| Original Residue | Substitutions | Exemplary Substitutions |
| Ala (A) | Val | Val; Leu; Ile |
| Arg (R) | Lys | Lys; Gln; Asn |
| Asn (N) | Gln | Gln; His; Asp, Lys; Arg |
| Asp (D) | Glu | Glu; Asn |
| Cys (C) | Ser | Ser; Ala |
| Gln (Q) | Asn | Asn; Glu |
| Glu (E) | Asp | Asp; Gln |
| Gly (G) | Ala | Ala |
| His (H) | Arg | Asn; Gln; Lys; Arg |
| Ile (I) | Leu | Leu; Val; Met; Ala; Phe; |
| | | Norleucine |
| Leu (L) | Ile | Norleucine; Ile; Val; Met; |
| | | Ala; Phe |
| Lys (K) | Arg | Arg; Gln; Asn |
| Met (M) | Leu | Leu; Phe; Ile |
| Phe (F) | Tyr | Leu; Val; Ile; Ala; Tyr |
| Pro (P) | Ala | Ala |
| Ser (S) | Thr | Thr |
| Thr (T) | Ser | Ser |
| Trp (W) | Tyr | Tyr; Phe |
| Tyr (Y) | Phe | Trp; Phe; Thr; Ser |
| Val (V) | Leu | Ile; Leu; Met; Phe; Ala; |
| | | Norleucine |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:
(1) Non-polar: Norleucine, Met, Ala, Val, Leu, Ile;
(2) Polar without charge: Cys, Ser, Thr, Asn, Gln;
(3) Acidic (negatively charged): Asp, Glu;
(4) Basic (positively charged): Lys, Arg;
(5) Residues that influence chain orientation: Gly, Pro; and
(6) Aromatic: Trp, Tyr, Phe, His.

Non-conservative substitutions are made by exchanging a member of one of these classes for another class.

Any cysteine residue not involved in maintaining the proper conformation of the antibody also may be substituted, generally with serine, to improve the oxidative stability of the molecule and prevent aberrant cross-linking. Conversely, cysteine bond(s) may be added to the antibody to improve its stability, particularly where the antibody is an antibody fragment such as an Fv fragment.

Amino acid modifications can range from changing or modifying one or more amino acids to complete redesign of a region, such as the variable region. Changes in the variable region can alter binding affinity and/or specificity. In some embodiments, no more than one to five conservative amino acid substitutions are made within a CDR domain. In other embodiments, no more than one to three conservative amino acid substitutions are made within a CDR domain. In still other embodiments, the CDR domain is CDR H3 and/or CDR L3.

Modifications also include glycosylated and nonglycosylated polypeptides, as well as polypeptides with other post-translational modifications, such as, for example, glycosylation with different sugars, acetylation, and phosphorylation. Antibodies are glycosylated at conserved positions in their constant regions (Jefferis and Lund, 1997, Chem. Immunol. 65:111-128; Wright and Morrison, 1997, TibTECH 15:26-32). The oligosaccharide side chains of the immunoglobulins affect the protein's function (Boyd et al., 1996, Mol. Immunol. 32:1311-1318; Wittwe and Howard, 1990, Biochem. 29:4175-4180) and the intramolecular interaction between portions of the glycoprotein, which can affect the conformation and presented three-dimensional surface of the glycoprotein (Jefferis and Lund, supra; Wyss and Wagner, 1996, Current Opin. Biotech. 7:409-416). Oligosaccharides may also serve to target a given glycoprotein to certain molecules based upon specific recognition structures. Glycosylation of antibodies has also been reported to affect antibody-dependent cellular cytotoxicity (ADCC). In particular, CHO cells with tetracycline-regulated expression of β(1,4)-N-acetylglucosaminyltransferase III (GnTIII), a glycosyltransferase catalyzing formation of bisecting GlcNAc, was reported to have improved ADCC activity (Umana et al., 1999, Mature Biotech. 17:176-180).

Glycosylation of antibodies is typically either N-linked or O-linked. N-linked refers to the attachment of the carbohydrate moiety to the side chain of an asparagine residue. The tripeptide sequences asparagine-X-serine, asparagine-X-threonine, and asparagine-X-cysteine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

The glycosylation pattern of antibodies may also be altered without altering the underlying nucleotide sequence. Glycosylation largely depends on the host cell used to express the antibody. Since the cell type used for expression of recombinant glycoproteins, e.g. antibodies, as potential therapeutics is rarely the native cell, variations in the glycosylation pattern of the antibodies can be expected (see, e.g. Hse et al., 1997, J. Biol. Chem. 272:9062-9070).

In addition to the choice of host cells, factors that affect glycosylation during recombinant production of antibodies include growth mode, media formulation, culture density, oxygenation, pH, purification schemes and the like. Various methods have been proposed to alter the glycosylation pattern achieved in a particular host organism including introducing or overexpressing certain enzymes involved in oligosaccharide production (U.S. Pat. Nos. 5,047,335; 5,510,261 and 5,278,299). Glycosylation, or certain types of glycosylation, can be enzymatically removed from the glycoprotein, for example, using endoglycosidase H (Endo H), N-glycosidase F, endoglycosidase F1, endoglycosidase F2, endoglycosidase F3. In addition, the recombinant host cell can be genetically engineered to be defective in processing certain types of polysaccharides. These and similar techniques are well known in the art.

Other methods of modification include using coupling techniques known in the art, including, but not limited to, enzymatic means, oxidative substitution and chelation. Modifications can be used, for example, for attachment of labels for immunoassay. Modified polypeptides are made using established procedures in the art and can be screened using standard assays known in the art, some of which are described below and in the Examples.

In some embodiments of the invention, the antibody comprises a modified constant region, such as a constant region that is immunologically inert or partially inert, e.g., does not trigger complement mediated lysis, does not stimulate ADCC, or does not activate microglia; or have reduced activities (compared to the unmodified antibody) in any one or more of the following: triggering complement mediated lysis, stimulating ADCC, or activating microglia. Different modifications of the constant region may be used to achieve optimal level and/or combination of effector functions. See, for example, Morgan et al., Immunology 86:319-324, 1995; Lund et al., J. Immunology 157:4963-9 157:4963-4969, 1996; Idusogie et al., J. Immunology 164:4178-4184, 2000; Tao et al., J. Immunology 143: 2595-2601, 1989; and Jefferis et al., Immunological Reviews 163:59-76, 1998. In some embodiments, the constant region is modified as described in Eur. J. Immunol., 1999, 29:2613-2624; PCT Application No. PCT/GB99/01441; and/or UK Patent Application No. 9809951.8. In other embodiments, the antibody comprises a human heavy chain IgG2 constant region comprising the following mutations: A330P331 to S330S331 (amino acid numbering with reference to the wild type IgG2 sequence). Eur. J. Immunol., 1999, 29:2613-2624. In still other embodiments, the constant region is aglycosylated for N-linked glycosylation. In some embodiments, the constant region is aglycosylated for N-linked glycosylation by mutating the glycosylated amino acid residue or flanking residues that are part of the N-glycosylation recognition sequence in the constant region. For example, N-glycosylation site N297 may be mutated to A, Q, K, or H. See, Tao et al., J. Immunology 143: 2595-2601, 1989; and Jefferis et al., Immunological Reviews 163:59-76, 1998. In some embodiments, the constant region is aglycosylated for N-linked glycosylation. The constant region may be aglycosylated for N-linked glycosylation enzymatically (such as removing carbohydrate by enzyme PNGase), or by expression in a glycosylation deficient host cell.

Other antibody modifications include antibodies that have been modified as described in PCT Publication No. WO 99/58572. These antibodies comprise, in addition to a binding domain directed at the target molecule, an effector domain having an amino acid sequence substantially homologous to all or part of a constant domain of a human immunoglobulin heavy chain. These antibodies are capable of binding the target molecule without triggering significant complement dependent lysis, or cell-mediated destruction of the target. In some embodiments, the effector domain is capable of specifically binding FcRn and/or FcγRIIb. These are typically based on chimeric domains derived from two or more human immunoglobulin heavy chain C_{H}2 domains. Antibodies modified in this manner are particularly suitable for use in chronic antibody therapy, to avoid inflammatory and other adverse reactions to conventional antibody therapy.

The invention includes affinity matured embodiments. For example, affinity matured antibodies can be produced by procedures known in the art (Marks et al., 1992, Bio/Technology, 10:779-783; Barbas et al., 1994, Proc Nat. Acad. Sci, USA 91:3809-3813; Schier et al., 1995, Gene, 169:147-155; Yelton et al., 1995, J. Immunol., 155:1994-2004; Jackson et al., 1995, J. Immunol., 154(7):3310-9; Hawkins et al., 1992, J. Mol. Biol., 226:889-896; and PCT Publication No. WO2004/058184).

The following methods may be used for adjusting the affinity of an antibody and for characterizing a CDR. One way of characterizing a CDR of an antibody and/or altering (such as improving) the binding affinity of a polypeptide, such as an antibody, termed “library scanning mutagenesis”. Generally, library scanning mutagenesis works as follows. One or more amino acid positions in the CDR are replaced with two or more (such as 3, 4, 5, 6, 7, 8, 9, 10, 11, 12, 13, 14, 15, 16, 17, 18, 19, or 20) amino acids using art recognized methods. This generates small libraries of clones (in some embodiments, one for every amino acid position that is analyzed), each with a complexity of two or more members (if two or more amino acids are substituted at every position). Generally, the library also includes a clone comprising the native (unsubstituted) amino acid. A small number of clones, e.g., about 20-80 clones (depending on the complexity of the library), from each library are screened for binding affinity to the target polypeptide (or other binding target), and candidates with increased, the same, decreased, or no binding are identified. Methods for determining binding affinity are well-known in the art. Binding affinity may be determined using Biacore surface plasmon resonance analysis, which detects differences in binding affinity of about 2-fold or greater. Biacore is particularly useful when the starting antibody already binds with a relatively high affinity, for example a K_{D} of about 10 nM or lower. Screening using Biacore surface plasmon resonance is described in the Examples, herein.

Binding affinity may be determined using Kinexa Biocensor, scintillation proximity assays, ELISA, ORIGEN immunoassay (IGEN), fluorescence quenching, fluorescence transfer, and/or yeast display. Binding affinity may also be screened using a suitable bioassay.

In some embodiments, every amino acid position in a CDR is replaced (in some embodiments, one at a time) with all 20 natural amino acids using art recognized mutagenesis methods (some of which are described herein). This generates small libraries of clones (in some embodiments, one for every amino acid position that is analyzed), each with a complexity of 20 members (if all 20 amino acids are substituted at every position).

In some embodiments, the library to be screened comprises substitutions in two or more positions, which may be in the same CDR or in two or more CDRs. Thus, the library may comprise substitutions in two or more positions in one CDR. The library may comprise substitution in two or more positions in two or more CDRs. The library may comprise substitution in 3, 4, 5, or more positions, said positions found in two, three, four, five or six CDRs. The substitution may be prepared using low redundancy codons. See, e.g., Table 2 of Balint et al., 1993, Gene 137(1):109-18).

The CDR may be CDRH3 and/or CDRL3. The CDR may be one or more of CDRL1, CDRL2, CDRL3, CDRH1, CDRH2, and/or CDRH3. The CDR may be a Kabat CDR, a Chothia CDR, or an extended CDR.

Candidates with improved binding may be sequenced, thereby identifying a CDR substitution mutant which results in improved affinity (also termed an “improved” substitution). Candidates that bind may also be sequenced, thereby identifying a CDR substitution which retains binding.

Multiple rounds of screening may be conducted. For example, candidates (each comprising an amino acid substitution at one or more position of one or more CDR) with improved binding are also useful for the design of a second library containing at least the original and substituted amino acid at each improved CDR position (i.e., amino acid position in the CDR at which a substitution mutant showed improved binding).

Preparation, and screening or selection of this library is discussed further below.

Library scanning mutagenesis also provides a means for characterizing a CDR, in so far as the frequency of clones with improved binding, the same binding, decreased binding or no binding also provide information relating to the importance of each amino acid position for the stability of the antibody-antigen complex. For example, if a position of the CDR retains binding when changed to all 20 amino acids, that position is identified as a position that is unlikely to be required for antigen binding. Conversely, if a position of CDR retains binding in only a small percentage of substitutions, that position is identified as a position that is important to CDR function. Thus, the library scanning mutagenesis methods generate information regarding positions in the CDRs that can be changed to many different amino acids (including all 20 amino acids), and positions in the CDRs which cannot be changed or which can only be changed to a few amino acids.

Candidates with improved affinity may be combined in a second library, which includes the improved amino acid, the original amino acid at that position, and may further include additional substitutions at that position, depending on the complexity of the library that is desired, or permitted using the desired screening or selection method. In addition, if desired, adjacent amino acid position can be randomized to at least two or more amino acids. Randomization of adjacent amino acids may permit additional conformational flexibility in the mutant CDR, which may, in turn, permit or facilitate the introduction of a larger number of improving mutations. The library may also comprise substitutions at positions that did not show improved affinity in the first round of screening.

The second library is screened or selected for library members with improved and/or altered binding affinity using any method known in the art, including screening using Biacore surface plasmon resonance analysis, and selection using any method known in the art for selection, including phage display, yeast display, and ribosome display.

The invention also encompasses fusion proteins comprising one or more fragments or regions from the antibodies or polypeptides of this invention. In some embodiments, a fusion polypeptide is provided that comprises amino acids of the variable light chain region shown in SEQ ID NO: 20 and/or the amino acids of the variable heavy chain region shown in SEQ ID NO: 21. In some embodiments, a fusion polypeptide is provided that comprises at least 10 contiguous amino acids of the variable light chain region shown in SEQ ID NO: 7 and/or at least 10 amino acids of the variable heavy chain region shown in SEQ ID NO: 8. In some embodiments, a fusion polypeptide is provided that comprises at least 10 contiguous amino acids of the variable light chain region shown in SEQ ID NO: 173 and/or at least 10 amino acids of the variable heavy chain region shown in SEQ ID NO: 12. In some embodiments, a fusion polypeptide is provided that comprises at least 10 contiguous amino acids of the variable light chain region shown in SEQ ID NO: 176 and/or at least 10 amino acids of the variable heavy chain region shown in SEQ ID NO: 12. In other embodiments, a fusion polypeptide is provided that comprises at least about 10, at least about 15, at least about 20, at least about 25, or at least about 30 contiguous amino acids of the variable light chain region and/or at least about 10, at least about 15, at least about 20, at least about 25, or at least about 30 contiguous amino acids of the variable heavy chain region. In another embodiment, the fusion polypeptide comprises a light chain variable region and/or a heavy chain variable region, as shown in any of the sequence pairs selected from among SEQ ID NOs: 7, 8, 9, 10, 11, 12, 13, 14, 15 and 16. In another embodiment, the fusion polypeptide comprises one or more CDR(s). In still other embodiments, the fusion polypeptide comprises CDR H3 (VH CDR3) and/or CDR L3 (VL CDR3). For purposes of this invention, a fusion protein contains one or more antibodies and another amino acid sequence to which it is not attached in the native molecule, for example, a heterologous sequence or a homologous sequence from another region. Exemplary heterologous sequences include, but are not limited to a “tag” such as a FLAG tag or a 6H is tag. Tags are well known in the art.

A fusion polypeptide can be created by methods known in the art, for example, synthetically or recombinantly. Typically, the fusion proteins of this invention are made by preparing and expressing a polynucleotide encoding them using recombinant methods described herein, although they may also be prepared by other means known in the art, including, for example, chemical synthesis.

This invention also provides compositions comprising antibodies or polypeptides conjugated (for example, linked) to an agent that facilitates coupling to a solid support (such as biotin or avidin). For simplicity, reference will be made generally to antibodies with the understanding that these methods apply to any of the TrkB binding and/or agonist embodiments described herein. Conjugation generally refers to linking these components as described herein. The linking (which is generally fixing these components in proximate association at least for administration) can be achieved in any number of ways. For example, a direct reaction between an agent and an antibody is possible when each possesses a substituent capable of reacting with the other. For example, a nucleophilic group, such as an amino or sulfhydryl group, on one may be capable of reacting with a carbonyl-containing group, such as an anhydride or an acid halide, or with an alkyl group containing a good leaving group (e.g., a halide) on the other.

An antibody or polypeptide of this invention may be linked to a labeling agent such as a fluorescent molecule, a radioactive molecule or any others labels known in the art. Labels are known in the art which generally provide (either directly or indirectly) a signal.

The invention also provides compositions (including pharmaceutical compositions) and kits comprising, as this disclosure makes clear, any or all of the antibodies and/or polypeptides described herein.

The invention also provides isolated polynucleotides encoding the antibodies of the invention, and vectors and host cells comprising the polynucleotides.

Accordingly, the invention provides polynucleotides (or compositions, including pharmaceutical compositions), comprising polynucleotides encoding any of the following: the antibody C2 or any fragment or part thereof having the ability to agonize TrkB. Also provided are polynucleotides (or compositions, including pharmaceutical compositions), comprising polynucleotides encoding any of the following: the antibody A2 or any fragment or part thereof having the ability to agonize TrkB.

In another aspect, the invention provides polynucleotides encoding any of the antibodies (including antibody fragments) and polypeptides described herein, such as antibodies and polypeptides having impaired effector function. Polynucleotides can be made and expressed by procedures known in the art.

In another aspect, the invention provides compositions (such as a pharmaceutical compositions) comprising any of the polynucleotides of the invention. In some embodiments, the composition comprises an expression vector comprising a polynucleotide encoding the antibody as described herein. In other embodiment, the composition comprises an expression vector comprising a polynucleotide encoding any of the antibodies or polypeptides described herein. In other embodiments, the composition comprises either or both of the polynucleotides encoding the amino acid sequences shown in SEQ ID NO: 20 and SEQ ID NO: 21. In other embodiments, the composition comprises either or both of the polynucleotides encoding the amino acid sequences shown in SEQ ID NO: 173 and SEQ ID NO: 12. In other embodiments, the composition comprises either or both of the polynucleotides encoding the amino acid sequences shown in SEQ ID NO: 176 and SEQ ID NO: 12. In still other embodiments, the composition comprises either or both of the polynucleotides encoding the amino acid sequences shown in SEQ ID NO: 7 and SEQ ID NO: 8. Expression vectors and administration of polynucleotide compositions are further described herein.

In another aspect, the invention provides a method of making any of the polynucleotides described herein.

Polynucleotides complementary to any such sequences are also encompassed by the present invention. Polynucleotides may be single-stranded (coding or antisense) or double-stranded, and may be DNA (genomic, cDNA or synthetic) or RNA molecules. RNA molecules include HnRNA molecules, which contain introns and correspond to a DNA molecule in a one-to-one manner, and mRNA molecules, which do not contain introns. Additional coding or non-coding sequences may, but need not, be present within a polynucleotide of the present invention, and a polynucleotide may, but need not, be linked to other molecules and/or support materials.

Polynucleotides may comprise a native sequence (i.e., an endogenous sequence that encodes an antibody or a portion thereof) or may comprise a variant of such a sequence. Polynucleotide variants contain one or more substitutions, additions, deletions and/or insertions such that the immunoreactivity of the encoded polypeptide is not diminished, relative to a native immunoreactive molecule. The effect on the immunoreactivity of the encoded polypeptide may generally be assessed as described herein. Variants preferably exhibit at least about 70% identity, more preferably, at least about 80% identity, yet more preferably, at least about 90% identity, and most preferably, at least about 95% identity to a polynucleotide sequence that encodes a native antibody or a portion thereof.

Two polynucleotide or polypeptide sequences are said to be “identical” if the sequence of nucleotides or amino acids in the two sequences is the same when aligned for maximum correspondence as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, or 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O., 1978, A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J., 1990, Unified Approach to Alignment and Phylogenes pp. 626-645 Methods in Enzymology vol. 183, Academic Press, Inc., San Diego, Calif.; Higgins, D. G. and Sharp, P. M., 1989, CABIOS 5:151-153; Myers, E. W. and Muller W., 1988, CABIOS 4:11-17; Robinson, E. D., 1971, Comb. Theor. 11:105; Santou, N., Nes, M., 1987, Mol. Biol. Evol. 4:406-425; Sneath, P. H. A. and Sokal, R. R., 1973, Numerical Taxonomy the Principles and Practice of Numerical Taxonomy, Freeman Press, San Francisco, Calif.; Wilbur, W. J. and Lipman, D. J., 1983, Proc. Natl. Acad. Sci. USA 80:726-730.

Preferably, the “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residue occurs in both sequences to yield the number of matched positions, dividing the number of matched positions by the total number of positions in the reference sequence (i.e. the window size) and multiplying the results by 100 to yield the percentage of sequence identity.

Variants may also, or alternatively, be substantially homologous to a native gene, or a portion or complement thereof. Such polynucleotide variants are capable of hybridizing under moderately stringent conditions to a naturally occurring DNA sequence encoding a native antibody (or a complementary sequence).

Suitable “moderately stringent conditions” include prewashing in a solution of 5×SSC, 0.5% SDS, 1.0 mM EDTA (pH 8.0); hybridizing at 50° C.-65° C., 5×SSC, overnight; followed by washing twice at 65° C. for 20 minutes with each of 2×, 0.5× and 0.2×SSC containing 0.1% SDS.

As used herein, “highly stringent conditions” or “high stringency conditions” are those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C. The skilled artisan will recognize how to adjust the temperature, ionic strength, etc., as necessary to accommodate factors such as probe length and the like.

It will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the present invention. Further, alleles of the genes comprising the polynucleotide sequences provided herein are within the scope of the present invention. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

The polynucleotides of this invention can be obtained using chemical synthesis, recombinant methods, or PCR. Methods of chemical polynucleotide synthesis are well known in the art and need not be described in detail herein. One of skill in the art can use the sequences provided herein and a commercial DNA synthesizer to produce a desired DNA sequence.

For preparing polynucleotides using recombinant methods, a polynucleotide comprising a desired sequence can be inserted into a suitable vector, and the vector in turn can be introduced into a suitable host cell for replication and amplification, as further discussed herein. Polynucleotides may be inserted into host cells by any means known in the art. Cells are transformed by introducing an exogenous polynucleotide by direct uptake, endocytosis, transfection, F-mating or electroporation. Once introduced, the exogenous polynucleotide can be maintained within the cell as a non-integrated vector (such as a plasmid) or integrated into the host cell genome. The polynucleotide so amplified can be isolated from the host cell by methods well known within the art. See, e.g., Sambrook et al., 1989, supra.

Alternatively, PCR allows reproduction of DNA sequences. PCR technology is well known in the art and is described in U.S. Pat. Nos. 4,683,195, 4,800,159, 4,754,065 and 4,683,202, as well as PCR: The Polymerase Chain Reaction, Mullis et al. eds., Birkauswer Press, Boston, 1994.

RNA can be obtained by using the isolated DNA in an appropriate vector and inserting it into a suitable host cell. When the cell replicates and the DNA is transcribed into RNA, the RNA can then be isolated using methods well known to those of skill in the art, as set forth in Sambrook et al., 1989, supra, for example.

Suitable cloning vectors may be constructed according to standard techniques, or may be selected from a large number of cloning vectors available in the art. While the cloning vector selected may vary according to the host cell intended to be used, useful cloning vectors will generally have the ability to self-replicate, may possess a single target for a particular restriction endonuclease, and/or may carry genes for a marker that can be used in selecting clones containing the vector. Suitable examples include plasmids and bacterial viruses, e.g., pUC18, pUC19, Bluescript (e.g., pBS SK+) and its derivatives, mp 18, mp 19, pBR322, pMB9, ColE1, pCR1, RP4, phage DNAs, and shuttle vectors such as pSA3 and pAT28. These and many other cloning vectors are available from commercial vendors such as BioRad, Strategene, and Invitrogen.

Expression vectors generally are replicable polynucleotide constructs that contain a polynucleotide according to the invention. It is implied that an expression vector must be replicable in the host cells either as episomes or as an integral part of the chromosomal DNA. Suitable expression vectors include but are not limited to plasmids, viral vectors, including adenoviruses, adeno-associated viruses, retroviruses, cosmids, and expression vector(s), as disclosed, e.g., in PCT Publication No. WO 87/04462. Vector components may generally include, but are not limited to, one or more of the following: a signal sequence; an origin of replication; one or more marker genes; suitable transcriptional controlling elements (such as promoters, enhancers and terminator). For expression (i.e., translation), one or more translational controlling elements are also usually required, such as ribosome binding sites, translation initiation sites, and stop codons.

The vectors containing the polynucleotides of interest can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides will often depend on features of the host cell.

The invention also provides host cells comprising any of the polynucleotides described herein. Any host cells capable of over-expressing heterologous DNAs can be used for the purpose of isolating the genes encoding the antibody, polypeptide or protein of interest. Non-limiting examples of mammalian host cells include but not limited to COS, HeLa, and CHO cells. See also PCT Publication No. WO 87/04462. Suitable non-mammalian host cells include prokaryotes (such as E. coli or B. subtillis) and yeast (such as S. cerevisae, S. pombe; or K. lactis). Preferably, the host cells express the cDNAs at a level of about 5 fold higher, more preferably, 10 fold higher, even more preferably, 20 fold higher than that of the corresponding endogenous antibody or protein of interest, if present, in the host cells. Screening the host cells for a specific binding to TrkB or a TrkB domain is effected by an immunoassay or FACS. A cell overexpressing the antibody or protein of interest can be identified.

### C. Compositions

The compositions used in the methods of the invention comprise an effective amount of a TrkB agonist antibody, a TrkB agonist antibody derived polypeptide, or other TrkB agonists described herein. Examples of such compositions, as well as how to formulate, are also described in an earlier section and below. In some embodiments, the composition further comprises a TrkB agonist. In another embodiment, the composition comprises one or more TrkB agonist antibodies. In other embodiments, the TrkB agonist antibody recognizes human TrkB. In still other embodiments, the TrkB agonist antibody is humanized. In yet other embodiments, the TrkB agonist antibody comprises a constant region that does not trigger an unwanted or undesirable immune response, such as antibody-mediated lysis or ADCC. In other embodiments, the TrkB agonist antibody comprises one or more CDR(s) of the antibody (such as one, two, three, four, five, or, in some embodiments, all six CDRs). In some embodiments, the TrkB agonist antibody is a human antibody.

It is understood that the compositions can comprise more than one TrkB agonist antibody (e.g., a mixture of TrkB agonist antibodies that recognize different epitopes of TrkB). Other exemplary compositions comprise more than one TrkB agonist antibodies that recognize the same epitope(s), or different species of TrkB agonist antibodies that bind to different epitopes of TrkB.

The composition used in the present invention can further comprise pharmaceutically acceptable carriers, excipients, or stabilizers (Remington: The Science and practice of Pharmacy 20th Ed., 2000, Lippincott Williams and Wilkins, Ed. K. E. Hoover), in the form of lyophilized formulations or aqueous solutions. Acceptable carriers, excipients, or stabilizers are nontoxic to recipients at the dosages and concentrations, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). Pharmaceutically acceptable excipients are further described herein.

The TrkB agonist antibody and compositions thereof can also be used in conjunction with other agents that serve to enhance and/or complement the effectiveness of the agents.

### D. Kits

The invention also provides kits for use in the instant methods. Kits of the invention include one or more containers comprising a TrkB agonist antibody (such as a humanized antibody) or peptide described herein and instructions for use in accordance with any of the methods of the invention described herein. Generally, these instructions comprise a description of administration of the TrkB agonist antibody for the above described therapeutic treatments.

In some embodiments, the antibody is a humanized antibody. In some embodiments, the antibody is human. In other embodiments, the antibody is a monoclonal antibody. The instructions relating to the use of a TrkB agonist antibody generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example, the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is a TrkB agonist antibody. The container may further comprise a second pharmaceutically active agent.

Kits may optionally provide additional components such as buffers and interpretive information. Normally, the kit comprises a container and a label or package insert(s) on or associated with the container.

### Mutations and Modifications

To express the TrkB antibodies of the present invention, DNA fragments encoding V_{H} (heavy chain variable region) and V_{L} (light chain variable region) can first be obtained using any of the methods described above. Various modifications, e.g. mutations, deletions, and/or additions can also be introduced into the DNA sequences using standard methods known to those of skill in the art. For example, mutagenesis can be carried out using standard methods, such as PCR-mediated mutagenesis, in which the mutated nucleotides are incorporated into the PCR primers such that the PCR product contains the desired mutations or site-directed mutagenesis.

One type of substitution, for example, that may be made is to change one or more cysteines in the antibody, which may be chemically reactive, to another residue, such as, without limitation, alanine or serine. For example, there can be a substitution of a non-canonical cysteine. The substitution can be made in a CDR or framework region of a variable domain or in the constant domain of an antibody. In some embodiments, the cysteine is canonical.

The antibodies may also be modified, e.g., in the variable domains of the heavy and/or light chains, e.g., to alter a binding property of the antibody. For example, a mutation may be made in one or more of the CDR regions to increase or decrease the K_{D} of the antibody for TrkB, to increase or decrease k_{off}, or to alter the binding specificity of the antibody. Techniques in site-directed mutagenesis are well-known in the art. See, e.g., Sambrook et al. and Ausubel et al., supra.

A modification or mutation may also be made in a framework region or constant domain to increase the half-life of a TrkB antibody. See, e.g., PCT Publication No. WO 00/09560. A mutation in a framework region or constant domain can also be made to alter the immunogenicity of the antibody, to provide a site for covalent or non-covalent binding to another molecule, or to alter such properties as complement fixation, FcR binding and antibody-dependent cell-mediated cytotoxicity. According to the invention, a single antibody may have mutations in any one or more of the CDRs or framework regions of the variable domain or in the constant domain.

In a process known as “germlining”, certain amino acids in the V_{H} and V_{L} sequences can be mutated to match those found naturally in germline V_{H} and V_{L} sequences. In particular, the amino acid sequences of the framework regions in the V_{H} and V_{L} sequences can be mutated to match the germline sequences to reduce the risk of immunogenicity when the antibody is administered. Germline DNA sequences for human V_{H} and V_{L} genes are known in the art (see e.g., the “Vbase” human germline sequence database; see also Kabat, E. A., et al., 1991, Sequences of Proteins of Immunological Interest, Fifth Edition, U.S. Department of Health and Human Services, NIH Publication No. 91-3242; Tomlinson et al., 1992, J. Mol. Biol. 227:776-798; and Cox et al., 1994, Eur. J. Immunol. 24:827-836).

Another type of amino acid substitution that may be made is to remove potential proteolytic sites in the antibody. Such sites may occur in a CDR or framework region of a variable domain or in the constant domain of an antibody. Substitution of cysteine residues and removal of proteolytic sites may decrease the risk of heterogeneity in the antibody product and thus increase its homogeneity. Another type of amino acid substitution is to eliminate asparagine-glycine pairs, which form potential deamidation sites, by altering one or both of the residues. In another example, the C-terminal lysine of the heavy chain of a TrkB antibody of the invention can be cleaved. In various embodiments of the invention, the heavy and light chains of the TrkB antibodies may optionally include a signal sequence.

Once DNA fragments encoding the V_{H} and V_{L} segments of the present invention are obtained, these DNA fragments can be further manipulated by standard recombinant DNA techniques, for example, to convert the variable region genes to full-length antibody chain genes, to Fab fragment genes, or to a scFv gene. In these manipulations, a V_{L}- or V_{H}-encoding DNA fragment is operatively linked to another DNA fragment encoding another protein, such as an antibody constant region or a flexible linker. The term “operatively linked”, as used in this context, is intended to mean that the two DNA fragments are joined such that the amino acid sequences encoded by the two DNA fragments remain in-frame.

The isolated DNA encoding the V_{H} region can be converted to a full-length heavy chain gene by operatively linking the V_{H}-encoding DNA to another DNA molecule encoding heavy chain constant regions (CH1, CH2 and CH3). The sequences of human heavy chain constant region genes are known in the art (see e.g., Kabat, E. A., et al., 1991, Sequences of Proteins of Immunological Interest, Fifth Edition, U.S. Department of Health and Human Services, NIH Publication No. 91-3242) and DNA fragments encompassing these regions can be obtained by standard PCR amplification. The heavy chain constant region can be an IgG1, IgG2, IgG3, IgG4, IgA, IgE, IgM or IgD constant region, but most preferably is an IgG1 or IgG2 constant region. The IgG constant region sequence can be any of the various alleles or allotypes known to occur among different individuals, such as Gm(1), Gm(2), Gm(3), and Gm(17). These allotypes represent naturally occurring amino acid substitution in the IgG1 constant regions. For a Fab fragment heavy chain gene, the V_{H}-encoding DNA can be operatively linked to another DNA molecule encoding only the heavy chain CH1 constant region. The CH1 heavy chain constant region may be derived from any of the heavy chain genes.

The isolated DNA encoding the V_{L} region can be converted to a full-length light chain gene (as well as a Fab light chain gene) by operatively linking the V_{L}-encoding DNA to another DNA molecule encoding the light chain constant region, C_{L}. The sequences of human light chain constant region genes are known in the art (see e.g., Kabat, E. A., et al., 1991, Sequences of Proteins of Immunological Interest, Fifth Edition, U.S. Department of Health and Human Services, NIH Publication No. 91-3242) and DNA fragments encompassing these regions can be obtained by standard PCR amplification. The light chain constant region can be a kappa or lambda constant region. The kappa constant region may be any of the various alleles known to occur among different individuals, such as Inv(1), Inv(2), and Inv(3). The lambda constant region may be derived from any of the three lambda genes.

To create a scFv gene, the V_{H}- and V_{L}-encoding DNA fragments are operatively linked to another fragment encoding a flexible linker, e.g., encoding the amino acid sequence (Gly₄-Ser)₃, such that the V_{H} and V_{L} sequences can be expressed as a contiguous single-chain protein, with the V_{L} and V_{H} regions joined by the flexible linker (See e.g., Bird et al., 1988, Science 242:423-426; Huston et al., 1988, Proc. Natl. Acad. Sci. USA 85:5879-5883; McCafferty et al., 1990, Nature 348:552-554.) The single chain antibody may be monovalent, if only a single V_{H} and V_{L} are used, bivalent, if two V_{H} and V_{L} are used, or polyvalent, if more than two V_{H} and V_{L} are used. Bispecific or polyvalent antibodies may be generated that bind specifically to TrkB and to another molecule.

In another embodiment, a fusion antibody or immunoadhesin may be made that comprises all or a portion of a TrkB antibody of the invention linked to another polypeptide. In another embodiment, only the variable domains of the TrkB antibody are linked to the polypeptide. In another embodiment, the V_{H} domain of a TrkB antibody is linked to a first polypeptide, while the V_{L} domain of a TrkB antibody is linked to a second polypeptide that associates with the first polypeptide in a manner such that the V_{H} and V_{L} domains can interact with one another to form an antigen binding site. In another preferred embodiment, the V_{H} domain is separated from the V_{L} domain by a linker such that the V_{H} and V_{L} domains can interact with one another. The V_{H}-linker-V_{L} antibody is then linked to the polypeptide of interest. In addition, fusion antibodies can be created in which two (or more) single-chain antibodies are linked to one another. This is useful if one wants to create a divalent or polyvalent antibody on a single polypeptide chain, or if one wants to create a bispecific antibody.

In other embodiments, other modified antibodies may be prepared using TrkB antibody encoding nucleic acid molecules. For instance, “Kappa bodies” (III et al., 1997, Protein Eng. 10:949-57), “Minibodies” (Martin et al., 1994, EMBO J. 13:5303-9), “Diabodies” (Holliger et al., 1993, Proc. Natl. Acad. Sci. USA 90:6444-6448), or “Janusins” (Traunecker et al., 1991, EMBO J. 10:3655-3659 and Traunecker et al., 1992, Int. J. Cancer (Suppl.) 7:51-52) may be prepared using standard molecular biological techniques following the teachings of the specification.

Bispecific antibodies or antigen-binding fragments can be produced by a variety of methods including fusion of hybridomas or linking of Fab′ fragments. See, e.g., Songsivilai & Lachmann, 1990, Clin. Exp. Immunol. 79:315-321, Kostelny et al., 1992, J. Immunol. 148:1547-1553. In addition, bispecific antibodies may be formed as “diabodies” or “Janusins.” In some embodiments, the bispecific antibody binds to two different epitopes of TrkB. In some embodiments, the modified antibodies described above are prepared using one or more of the variable domains or CDR regions from a human TrkB antibody provided herein.

Representative materials of the present invention were deposited in the American Type Culture Collection (ATCC) on Aug. 13, 2009, and were assigned the accession numbers in Table 5:

TABLE 5

| | ATCC | |
|---|---|---|
| Material | Accession No. | Date of Deposit |
| RN1026a-HC (RN1026A heavy chain) | PTA-10271 | Aug. 13, 2009 |
| RN1026a-LC (RN1026A light chain) | PTA-10272 | Aug. 13, 2009 |
| 4A6-HC (4A6 heavy chain) | PTA-10273 | Aug. 13, 2009 |
| 4A6-LC (4A6 light chain) | PTA-10274 | Aug. 13, 2009 |
| 4A6-12-HC (4A6-12 heavy chain) | PTA-10275 | Aug. 13, 2009 |
| C2-HC (C2 heavy chain) | PTA-10276 | Aug. 13, 2009 |
| C2-LC (C2 light chain) | PTA-10277 | Aug. 13, 2009 |
| A2-LC (A2 light chain) | PTA-10496 | Nov. 24, 2009 |

Vector RN1026a-HC is a polynucleotide encoding the RN1026A heavy chain variable region, and vector RN1026a-LC is a polynucleotide encoding the RNA1026A light chain variable region. Vector 4A6-HC is a polynucleotide encoding the 4A6 heavy chain variable region, and vector 4A6-LC is a polynucleotide encoding the 4A6 light chain variable region. Vector 4A6-12-HC is a polynucleotide encoding the 4A6-12 heavy chain variable region. Vector C2-HC is a polynucleotide encoding the C2 heavy chain variable region, and vector C2-LC is a polynucleotide encoding the C2 light chain variable region. Vector A2-LC is a polynucleotide encoding the A2 light chain variable region. The polynucleotide encoding the A2 heavy chain variable region is the same as the polynucleotide encoding the C2 heavy chain variable region.

The deposit was made under the provisions of the Budapest Treaty on the International Recognition of the Deposit of Microorganisms for the Purpose of Patent Procedure and Regulations thereunder (Budapest Treaty). This assures maintenance of a viable curlture of the deposit for 30 years from the date of deposit. The deposit will be made available by ATCC under the terms of the Budapest Treaty, and subject of an agreement between Pfizer, Inc. and ATCC, which assures permanent and unrestricted availability of the progeny of the culture of the deposit to the public upon issuance of the pertinent U.S. patent or upon laying open to the public of any U.S. or foreign patent application, whichever comes first, and assures availability of the progeny to one determined by the U.S. Commissioner of Patents and Trademarks to be entitled thereto according to 35 U.S.C. Section 122 and the Commissioner's rules pursuant thereto (including 37 C.F.R. Section 1.14 with particular reference to 8860G 638).

The assignee of the present application has agreed that if a culture of the materials on deposit should die or be lost or destroyed when cultivated under suitable conditions, the materials will be promptly replaced on notification with another of the same. Availability of the deposited material is not to be construed as a license to practice the invention in contravention of the rights granted under the authority of any government in accordance with its patent laws.

The following examples are offered for illustrative purposes only, and are not intended to limit the scope of the present invention in any way. Indeed, various modifications of the invention in addition to those shown and described herein will become apparent to those skilled in the art from the foregoing description and fall within the scope of the appended claims.

### EXAMPLES

### Example 1

### Humanized Anti-TrkB Antibodies

The murine monoclonal antibody 38B8 was humanized and affinity matured to provide the RN1026A, C2 and A2 antibodies. RN1026A has a K_{D} for human TrkB of 1.27 nM, a K_{D} for mouse TrkB of 2.27 nM, and a K_{D} for rat TrkB of 4.0 nM when measured by Biacore at 37° C. T_{1/2} for human TrkB is 4.53 min at 37° C. C2 has a K_{D} for human TrkB of 2.54 nM, a K_{D} for mouse TrkB of 1.44 nM, and a K_{D} for rat TrkB of 2.29 nM when measured by Biacore at 25° C. T_{1/2} for human TrkB is 2.9 min at 25° C. RN1026A and C2 show significantly greater efficacy than 38B8 in promoting mouse neuron survival (see, Examples below). C2 was tested for its ability to block BDNF using on an Octet® System. The results demonstrated that C2 blocks BDNF binding to TrkB.

The amino acid sequence of RN1026A fully humanized light chain variable region (SEQ ID NO: 7) is shown below:

(SEQ ID NO: 7)

DIQMTQSPSSLSASVGDRVTITCRASENVYSNLAWYQQKPGKAPKLLIYA
ASNLQSGVPSRFSGSGSGTDFTFTISSLQPEDIATYYCQHFWGSPFTFGQ
GTKLEIK

The amino acid sequence of RN1026A fully humanized heavy chain variable region (SEQ ID NO: 8) is shown below:

(SEQ ID NO: 8)

QVQLVQSGAEVKKPGASVKVSCKASGYTFTNYDIIWVRQAPGQGLEWMGY
INPYNRRREYNEKFKGRVTMTRDTSTSTVYMELSSLRSEDTAVYYCARLL
KYRRFRYYAIDYWGQGTTVTVSS

The amino acid sequence of C2 fully humanized light chain (SEQ ID NO: 174) is shown below:

(SEQ ID NO: 174)

DIQMTQSPSSLSASVGDRVTITCRTSENVYSNLAWYQQKPGKAPKLLIYA
ASNLQSGVPSRFSGSGSGTDYTFTISSLQPEDIATYYCQHFWGSPFTFGQ
GTKLEIKTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC

The amino acid sequence of C2 fully humanized heavy chain (SEQ ID NO: 175) is shown below:

(SEQ ID NO: 175)

QVQLVQSGAEVKKPGASVKVSCKASGYTFTNYDIIWVRQAPGQGLEWMGY
INPYNDGTKYNEKFKGRVTMTRDTSTSTVYMELSSLRSEDTAVYYCARLL
KYRRFRYYAIDYWGQGTTVTVSSASTKGPSVFPLAPCSRSTSESTAALGC
LVKDYFPEPVTVSWNSGALTSGVHTFPAVLQSSGLYSLSSVVTVPSSNFG
TQTYTCNVDHKPSNTKVDKTVAPPVAGPSVFLFPPKPKDTLMISRTPEVT
CVVVDVSHEDPEVQFNWYVDGVEVHNAKTKPREEQFNSTFRVVSVLTVVH
QDWLNGKEYKCKVSNKGLPSSIEKTISKTKGQPREPQVYTLPPSREEMTK
NQVSLTCLVKGFYPSDIAVEWESNGQPENNYKTTPPMLDSDGSFFLYSKL
TVDKSRWQQGNVFSCSVMHEALHNHYTQKSLSLSPGK

The amino acid sequence of A2 fully humanized light chain (SEQ ID NO: 178) is shown below:

(SEQ ID NO: 178)

DIQMTQSPSSLSASVGDRVTITCRASENVYSNLAWYQQKPGKAPKLLIYA
ASNLQSGVPSRFSGSGSGTDYTFTISSLQPEDIATYYCQHFWYSPWTFGQ
GTKLEIKTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC

The amino acid sequence of A2 fully humanized heavy chain variable region (SEQ ID NO: 175) is shown below:

(SEQ ID NO: 175)

QVQLVQSGAEVKKPGASVKVSCKASGYTFTNYDIIWVRQAPGQGLEWMGY
INPYNDGTKYNEKFKGRVTMTRDTSTSTVYMELSSLRSEDTAVYYCARLL
KYRRFRYYAIDYWGQGTTVTVSSASTKGPSVFPLAPCSRSTSESTAALGC
LVKDYFPEPVTVSWNSGALTSGVHTFPAVLQSSGLYSLSSVVTVPSSNFG
TQTYTCNVDHKPSNTKVDKTVAPPVAGPSVFLFPPKPKDTLMISRTPEVT
CVVVDVSHEDPEVQFNWYVDGVEVHNAKTKPREEQFNSTFRVVSVLTVVH
QDWLNGKEYKCKVSNKGLPSSIEKTISKTKGQPREPQVYTLPPSREEMTK
NQVSLTCLVKGFYPSDIAVEWESNGQPENNYKTTPPMLDSDGSFFLYSKL
TVDKSRWQQGNVFSCSVMHEALHNHYTQKSLSLSPGK

### Example 2

### Determining Antibody Binding Affinity

Determining binding affinity of humanized anti-TrkB antibodies may be performed by measuring the binding affinity of monofunctional Fab fragments of the antibody. To obtain monofunctional Fab fragments, an antibody (for example, IgG) can be cleaved with papain or expressed recombinantly. The affinity of an anti-TrkB Fab fragment of an antibody can be determined by surface plasmon resonance (BIAcore3000™ surface plasmon resonance (SPR) system, BIAcore, INC, Piscaway N.J.). CM5 chips can be activated with N-ethyl-N′-(3-dimethylaminopropyl)-carbodiinide hydrochloride (EDC) and N-hydroxysuccinimide (NHS) according to the supplier's instructions.

TrkB monomers (H is ECD) of human, mouse, and rat were amine-coupled to the chip at levels of 2008, 1008, and 889RU respectively in an effort to give low capacity surfaces suitable for kinetic analysis. Fabs (C1, C2, A2, 4A6, and 4B12) were diluted to 100 nM (according to their labeled concentrations by NanoDrop, using 1AU=0.6 mg/mL) to give the top concentration of a 3 fold serial dilution. Samples were injected from low to high concentration as a titration series for 84 sec, allowing a 10 minute dissociation phase for the 100 nM sample. Surfaces were regenerated with Pierce+salt and all titrations were duplicated to demonstrate that the assay was reproducible. The full-length mouse anti-TrkB antibody 38B8-IgG was used as a (+) control to confirm that the chip was active.

The concentrations of the Fab proteins are determined by ELISA and/or SDS-PAGE electrophoresis using a Fab of known concentration (as determined by amino acid analysis) as a standard. Kinetic association rates (kₒₙ) and dissociation rates (k_{off}) (generally measured at 25° C. or 37° C.) are obtained simultaneously by fitting the data to a 1:1 Langmuir binding model (Karlsson, R. Roos, H. Fagerstam, L. Petersson, B., 1994 Methods Enzymology 6:99-110) using the BIAevaluation program. Equilibrium dissociation constant (K_{D}) values are calculated as k_{off}/kₒₙ.

The results of Example 2 are shown in Tables 6 and 7. These data show that the antibodies have excellent affinity for human TrkB, rat TrkB or mouse TrkB as indicated.

TABLE 6

| | | Kₒₙ for TrkB at | K_{off} for TrkB at | K_{D} for TrkB at |
|---|---|---|---|---|
| mAb | ligand | 25° C. (1/Ms) | 25° C. (1/S) | 25° C. (nM) |
| C2 | human | 5.52 × 10⁵ | 1.4 × 10⁻³ | 2.54 |
| A2 | human | 1.34 × 10⁶ | 1.45 × 10⁻³ | 1.08 |
| 4A6 | human | 3.89 × 10⁶ | 7.05 × 10⁻⁴ | 0.18 |
| 4B12 | human | 2.5 × 10⁶ | 5.32 × 10⁻⁴ | 0.21 |
| C2 | mouse | 5.1 × 10⁵ | 7.34 × 10⁻⁴ | 1.44 |
| A2 | mouse | 1.24 × 10⁶ | 6.36 × 10⁻⁴ | 0.51 |
| 4A6 | mouse | 4.35 × 10⁶ | 9.24 × 10⁻⁵ | 0.02 |
| 4B12 | mouse | 2.38 × 10⁶ | 1.46 × 10⁻⁴ | 0.06 |
| C2 | rat | 6.38 × 10⁵ | 1.46 × 10⁻³ | 2.29 |
| A2 | rat | 1.29 × 10⁶ | 1.52 × 10⁻³ | 1.18 |
| 4A6 | rat | 2.45 × 10⁶ | 6.66 × 10⁻⁴ | 0.27 |
| 4B12 | rat | 1.99 × 10⁶ | 8.1 × 10⁻⁴ | 0.41 |

TABLE 7

| | | Kₒₙ for TrkB at | K_{off} for TrkB at | K_{D} for TrkB at |
|---|---|---|---|---|
| mAb | ligand | 37° C. (1/Ms) | 37° C. (1/S) | 37° C. (nM) |
| RN1026A | human | 2.00 × 10⁶ | 2.55 × 10⁻³ | 1.27 |
| 4A6 | human | 2.14 × 10⁶ | 3.72 × 10⁻⁴ | 0.17 |
| RN1026A | mouse | 1.75 × 10⁶ | 3.96 × 10⁻³ | 2.27 |
| 4A6 | mouse | 1.76 × 10⁶ | 6.60 × 10⁻⁴ | 0.37 |
| RN1026A | rat | 1.41 × 10⁶ | 9.24 × 10⁻³ | 4.0 |
| 4A6 | rat | 1.36 × 10⁶ | 1.33 × 10⁻³ | 0.48 |

### Example 3

### Humanized Agonist Anti-TrkB Antibodies Promote Neuron Survival

This example illustrates improved neuron survival by humanized agonist anti-TrkB antibodies in an E15 Nodose neuron survival assay.

The Nodose ganglion neurons obtained from E15 embryos were supported by BDNF, so that at saturating concentrations of the neurotrophic factor the survival was close to 100% after 48 hours in culture. In the absence of BDNF, less than 5% of the neurons survived after 48 hours. Therefore, the survival of E15 nodose neurons is a sensitive assay to evaluate the agonist activity of anti-TrkB antibodies, i.e., agonist antibodies will promote survival of E15 nodose neurons.

Time-mated pregnant Swiss Webster female mice were euthanized by CO2 inhalation. The uterine horns were removed and the embryos at embryonic stage E15 were extracted. The nodose ganglia were dissected, then trypsinized, mechanically dissociated, and plated at a density of 200-300 cells per well in defined, serum-free medium in 96-well plates coated with poly-L-ornithine and laminin. The agonist activity of anti-TrkB antibodies was evaluated in a dose-response manner in triplicates with reference to human BDNF. After 48 hours in culture, the cells were subjected to an automated immunocytochemistry protocol performed on a Biomek FX liquid handling workstation (Beckman Coulter). The protocol included fixation (4% formaldehyde, 5% sucrose, PBS), permeabilization (0.3% Triton X-100 in PBS), blocking of unspecific binding sites (5% normal goat serum, 0.1% BSA, PBS) and sequential incubation with a primary and secondary antibodies to detect neurons. A rabbit polyclonal antibody against the protein gene product 9.5 (PGP9.5, Chemicon), which was an established neuronal phenotypic marker, was used as primary antibody. Alexa Fluor 488 goat anti-rabbit (Molecular Probes) was used as secondary reagent together with the nuclear dye Hoechst 33342 (Molecular Probes) to label the nuclei of all the cells present in the culture. Image acquisition and image analysis were performed on a Discovery-1/GenII Imager (Universal Imaging Corporation). Images were automatically acquired at two wavelengths for Alexa Fluor 488 and Hoechst 33342, with the nuclear staining being used as reference point, since it is present in all the wells, for the image-based auto focus-system of the Imager. Appropriate objectives and number of sites imaged per well were selected to cover the entire surface of each well. Automated image analysis was set up to count the number of neurons present in each well after 48 hours in culture based on their specific staining with the anti-PGP9.5 antibody. Careful thresholding of the image and application of morphology and fluorescence intensity based selectivity filters resulted in an accurate count of neurons per well.

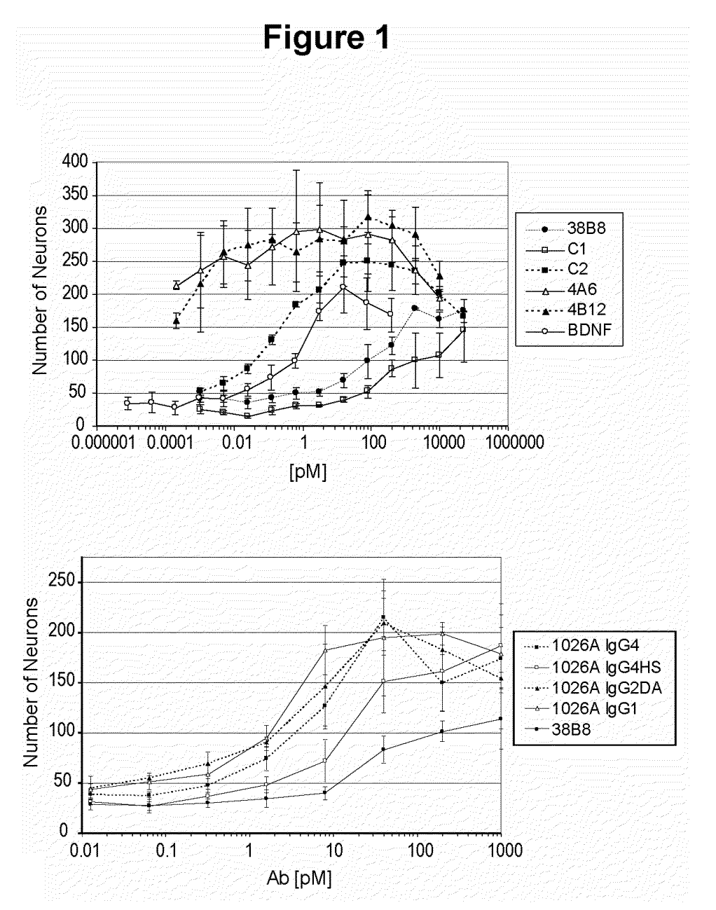

The top graph of FIG. 1 shows the effects of four humanized agonist anti-TrkB humanized antibodies C1, C2, 4A6 and 4B12 on mouse neuron survival. 38B8 is a mouse anti-TrkB agonist antibody. Humanized agonist anti-TrkB antibodies C2, 4A6 and 4B12 each show greater efficacy in promoting mouse neuron survival than the mouse antibody and BDNF (FIG. 1, top). The lower graph of FIG. 1 shows the effects of four different isotype versions of agonist anti-TrkB humanized antibody RN1026A: 1026A IgG4, 1026A IgG4HS (IgG4 with a stabilized hinge region), 1026A IgG2ΔA and 1026A IgG1. Again, 38B8 is a mouse anti-TrkB agonist antibody used as a positive control. Each of the four RN1026A antibodies shows significantly greater efficacy than 38B8 in promoting mouse neuron survival.

### Example 4

### Agonist Anti-TrkB Antibodies Improve Function in a Young Mouse Model of Charcot-Marie-Tooth Disease (CMT)

This example illustrates the efficacy of agonist anti-TrkB antibody, agonist anti-TrkC antibody or the combination of both in young trembler mice in a 32-week treatment protocol starting at their adolescent age.

Charcot-Marie-Tooth disease type 1A (CMT1A) is strongly linked to the CMT1A marker VAW409R3 on chromosome 17p11.2, and a point mutation in the peripheral myelin protein-22 (PMP-22) is completely linked with the disease. See, e.g., Valentijn et al., 1992 Nature Genetics 2: 288-291. The mutation, a proline for leucine substitution in the first putative transmembrane domain, is identical to that found in the Trembler-J (Tr-j)mouse (“trembler mouse”). Valentijn et al., 1992. The trembler mouse is a naturally occurring mutant with a point mutation in the PMP-22 gene causing severe peripheral nerve demyelination. It is a genetically homologous murine model for Charcot-Marie-Tooth disease type 1A (CMT 1A). See, Meekins et al., Muscle and Nerve 36(1):81-86, published online Apr. 18, 2007.

In experiments performed in support of the invention, six- to seven-week old trembler mice (Jackson Laboratory, Bar Harbor, Me., n=29) were divided into four treatment groups receiving an escalating dose of 0.5, 1 and then 2 mg/kg/week of various antibodies over 12 weeks, and then continued on 2 mg/kg/week for an additional 20 weeks. In this example, the agonist anti-TrkB antibody (TrkB AAb) used was the murine monoclonal antibody 38B8, and the agonist anti-TrkC antibody (TrkC AAb) used was a recombinant chimeric antibody having variable regions of the humanized TrkC antibody (clone A5) fused with the constant region of the rat IgG1 heavy chain and rat kappa light chain. The young trembler mice received TrkB AAb, TrkC AAb, TrkB AAb+TrkC AAb, or PBS according to the dosage regimen above.

Electrophysiology of sciatic nerve conduction was first collected at 12 weeks after the initiation of the study, i.e., at the end of the 0.5 to 2 mg/kg dose escalation period. During the full dose (2 mg/kg) period, i.e., 12-32 weeks of study, bilateral hindlimb grip strength was measured every other week. At the end of 32 weeks of study, the left hindlimb grip strength was measured twice daily (3 trials in each morning and afternoon session) for 3 consecutive days. The electrophysiological study of the ipsilateral (i.e., the same left) hindlimb was conducted to correlate with the grip strength data.

The compound muscle action potential (CMAP) and conduction velocity (CV) in the sciatic nerves were measured at both 12 and 32 weeks of treatment. Of all parameters measured in nerve conduction studies, CMAP is used to determine the status of motor nerves. CMAP has been demonstrated to be a valid parameter of nerve regeneration and re-innervation, correlating with the grip strength (Muscle Nerve 38: 1254-1265, 2008).

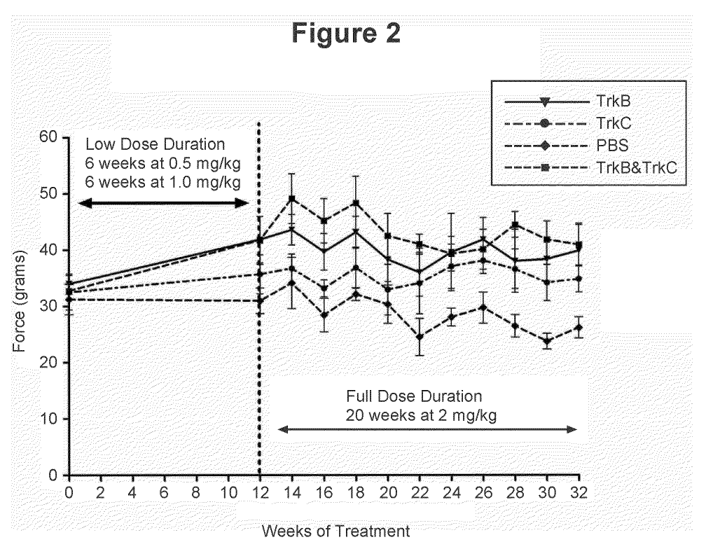

Bilateral hindlimb grip strength in mice that are agonist anti-TrkB antibody-treated, agonist anti-TrkC antibody-treated, or the combination of treatment of the two antibodies, were all significantly higher than that of the PBS treated group over the 12 to 32 weeks of treatment (FIG. 2). Grip strength of normal mice is about 49 grams as indicated by the left-hand side double-headed arrow in FIG. 2. Mice treated with TrkB AAb, TrkC AAb, or TrkB AAb+TrkC AAb showed significantly increased grip strength compared to treatment mice treated with PBS.

The CMAP and CV data from young trembler mice treated with various antibodies for 32 weeks are shown in Table 8. The data from PBS treated trembler mice are comparable to previous published values (Meekins et al., JPNS 9: 177-182, 2004). The CMAP Area, which is a highly accurate parameter reflecting the total number of myelinated axons, in mice treated with either agonist anti-TrkB antibody, agonist anti-TrkC antibody, or the combination of the two antibodies, were all significantly higher than that in the PBS treated group.

TABLE 8
CMAP and Conduction Velocity in the Sciatic Nerve
at 32 Weeks post-treatment

| | | Latency | Duration | Amp | Area | |
|---|---|---|---|---|---|---|
| Treatment | No. | (ms) | (ms) | (mV) | (mV · ms) | CV (m/s) |
| TrkB AAb | 7 | 2.43 ± 0.29 | 4.53 ± 1.37 | 0.40 ± 0.18 | 0.54 ± 0.24* | 13.09 ± 5.36 |
| TrkC AAb | 7 | 2.26 ± 0.35 | 4.47 ± 1.76 | 0.54 ± 0.24 | 0.60 ± 0.26** | 12.84 ± 4.31 |
| TrkB AAb + | 8 | 2.40 ± 0.50 | 5.84 ± 2.57 | 0.36 ± 0.11 | 0.60 ± 0.19*** | 14.93 ± 5.91 |
| TrkC AAb | | | | | | |
| PBS | 7 | 2.26 ± 0.34 | 4.07 ± 1.19 | 0.39 ± 0.16 | 0.36 ± 0.10 | 11.23 ± 3.47 |
| control | | | | | | |

*P = 0.064 vs. PBS,
**P = 0.0356 vs. PBS,
***P = 0.005 vs. PBS

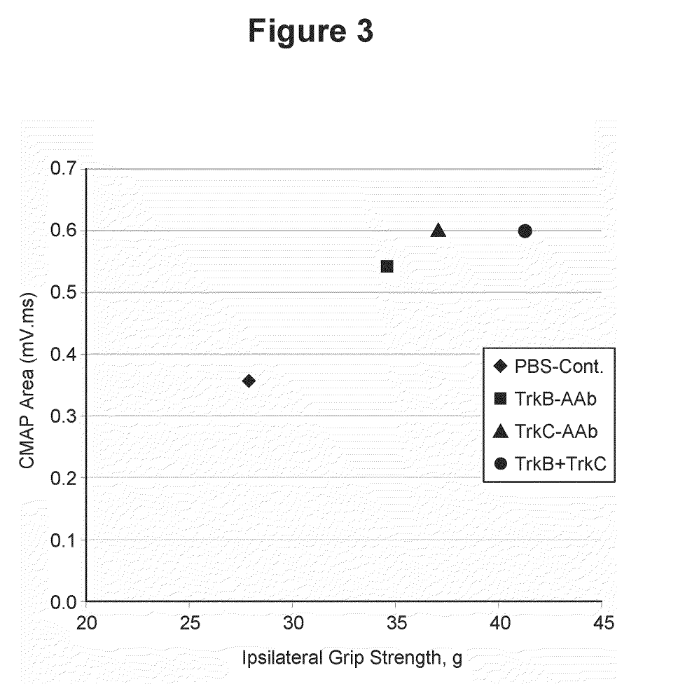

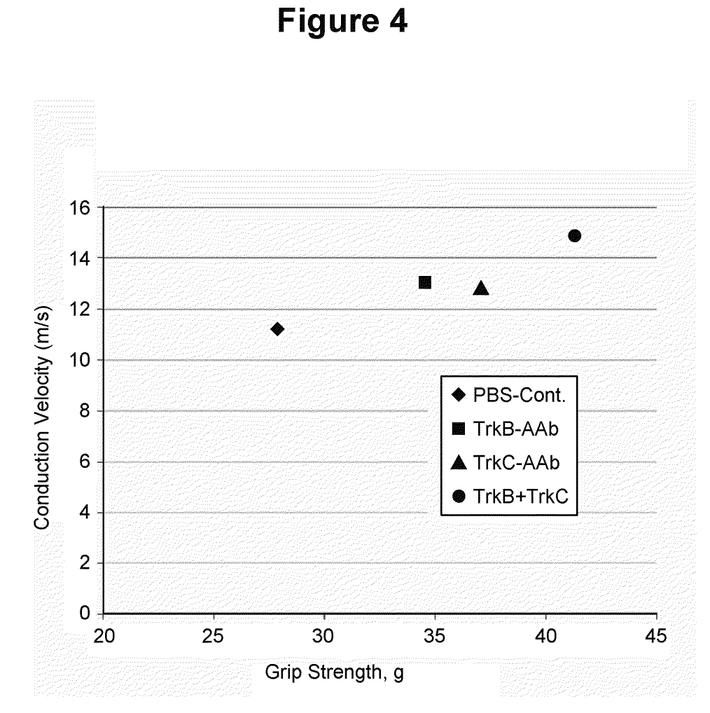

At the end of 32 weeks of treatment, the ipsilateral hindlimb grip strength positively correlated with the CMAP Area and CV for all three antibody treated groups (FIGS. 3 and 4).

Bilateral hindlimb grip strength in mice treated with agonist anti-TrkB antibody, agonist anti-TrkC antibody, or the combination of the two antibodies, were all significantly higher than that of the PBS treated group over the 12 to 32 weeks of treatment.

These data are consistent with results reported for NTs, demonstrating that the agonist anti-TrkB antibody affects neuronal cells in manner consistent with naturally-occurring TrkB agonist ligands.

### Example 5

### Agonist Anti-TrkB Antibodies Improve Function in an Adult Mouse Model of Charcot-Marie-Tooth Disease (CMT)

This example illustrates the efficacy of agonist anti-TrkB antibody, agonist anti-TrkC antibody or the combination of both in adult trembler mice in a 20-week treatment protocol starting at their adolescent age. In this example, the agonist anti-TrkB antibody (TrkB AAb) used was the murine monoclonal antibody 38B8, and the agonist anti-TrkC antibody (TrkC AAb) used was a recombinant chimeric antibody having variable regions of the humanized TrkC antibody (clone A5) fused with the constant region of the rat IgG1 heavy chain and rat kappa light chain. The adult trembler mice received TrkB AAb, TrkC AAb, TrkB AAb+TrkC AAb, or PBS.

In experiments performed in support of the invention, 11 to 12 week old trembler mice (Jackson Laboratory, Bar Harbor, Me.) were divided into four treatment groups receiving 2 mg/kg/week of various antibodies over 20 weeks.

During the treatment period, bilateral hindlimb grip strength was measured every other week. At the end of 20 weeks of treatment, the left hindlimb grip strength was measured twice daily (3 trials in each morning and afternoon session) for 3 consecutive days. Finally the electrophysiological study of the ipsilateral (i.e. the same left) hindlimb was conducted to correlate with the grip strength data.

The compound muscle action potential (CMAP) and conduction velocity (CV) data from adult trembler mice treated with various antibodies for 20 weeks are shown in Table 9. The CMAP Area, which is a highly accurate parameter reflecting the total number of myelinated axons, in TrkB AAb treated mice was significantly higher than that in the PBS treated group. The CMAP Area in mice treated with TrkC AAb or the combination of the two antibodies, was numerically higher than that of the PBS treated group, but the difference did not reach statistical significance, likely due to the small sample sizes of this study.

TABLE 9
CMAP and Conduction Velocity in the Sciatic Nerve

| Adult TrJ | | Latency | Duration | Amp | Area | |
|---|---|---|---|---|---|---|
| mice | No | (ms) | (ms) | (mV) | (mV · ms) | CV (m/s) |
| TrkB AAb | 5 | 2 ± 0.32 | 5.12 ± 1.64 | 0.57 ± 0.22* | 0.78 ± 0.28** | 13.44 ± 8.95 |
| TrkC AAb | 6 | 2.3 ± 0.36 | 4.52 ± 0.97 | 0.43 ± 0.21 | 0.58 ± 0.33 | 11.7 ± 2.92 |
| TrkB + TrkC | 4 | 2.25 ± 0.42 | 3.53 ± 0.62 | 0.56 ± 0.19 | 0.65 ± 0.37 | 12.53 ± 5.08 |
| PBS | 4 | 2.15 ± 0.17 | 4.15 ± 1.02 | 0.33 ± 0.13 | 0.4 ± 0.12 | 7.50 ± 1.75 |
| control | | | | | | |

*P = 0.048 vs. PBS,
**P = 0.019 vs. PBS

The age at the end of the treatment was the same for the young and the adult trembler studies, and the CMAP and CV measurements from these two studies were pooled and analyzed in Table 10. The CMAP Area measurements in mice treated with TrkB AAb, TrkC AAb or the combination of the two antibodies, were all significantly higher than that in the PBS treated group. In addition, the CVs in the TrkB+TrkC AAb combination treatment group were significantly higher than that in the PBS treated group.

TABLE 10
CMAP and Conduction Velocity in the Sciatic Nerve

| | | Latency | Duration | Amp | Area | CV |
|---|---|---|---|---|---|---|
| Pooled | No | (ms) | (ms) | (mV) | (mV · ms) | (m/s) |
| TrkB AAb | 12 | 2.25 ± 0.36 | 4.78 ± 1.45 | 0.47 ± 0.21 | 0.64 ± 0.27* | 13.23 ± 6.70 |
| TrkC AAb | 13 | 2.28 ± 0.34 | 4.49 ± 1.39 | 0.49 ± 0.22 | 0.59 ± 0.28*1 | 12.32 ± 3.63 |
| TrkB + TrkC | 12 | 2.35 ± 0.40 | 5.07 ± 2.27 | 0.43 ± 0.16 | 0.62 ± 0.24*2 | 14.13 ± 5.30*3 |
| PBS | 11 | 2.22 ± 0.28 | 4.10 ± 1.08 | 0.37 ± 0.14 | 0.37 ± 0.10 | 9.37 ± 3.63 |
| control | | | | | | |

*p = 0.002,
*1p = 0.011,
*2p = 0.003,
*3p = 0.017

### Example 6

### Agonist Anti-TrkB Antibody Treatment Resulted in Body Weight Change

This example illustrates the efficacy of agonist anti-TrkB antibody in high fat diet-induced obesity (D10) mice. In this example, animals were housed in a temperature and humidity controlled room with a 12 hour light/dark cycle, with ad libitum access to water and food unless otherwise specified for the pair-fed study. The DIO mice were produced as follows: male C57BL/6J mice (JAX West, West Sacramento, Calif.) were obtained at 6 weeks of age and immediately put on 58% high fat diet (D12331i, Research Diets) for 8 weeks. DIO mice were treated with 38B8, a mouse monoclonal antibody subtype IgG1 or treated with agonist anti-TrkB antibody C2. In this example, the C2 antibody used was an affinity matured recombinant chimeric antibody with human variable regions (sequences shown in Table 2 above) fused to mouse IgG1 constant region.

Mouse In Vivo Efficacy Determination

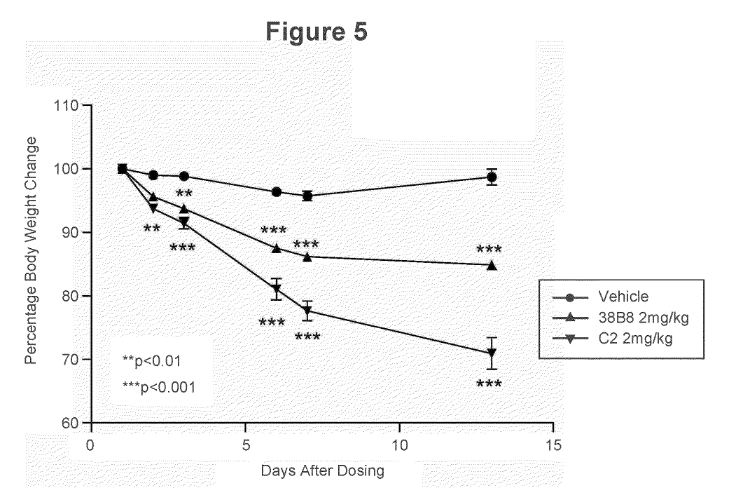

DIO mice were singly-housed and allowed to acclimate for one week. Body weight and measurements were taken prior to dosing. DIO mice were dosed intraperitoneally with either vehicle (n=6), 38B8 (n=6) or C2 (n=6) at 2 mg/kg. Body weight was measured at days 1, 2, 3, 6, 7, and 13 after dosing (FIG. 5).

The results of the in vivo efficacy study indicate that both 38B8 and C2 significantly reduce the body weight compared with vehicle (FIG. 5). A single intraperitoneal dose of C2 at 2 mg/kg caused a statistically significant body weight reduction compared to vehicle-treated mice as early as day 2 after dosing. A single intraperitoneal dose of 38B8 at 2 mg/kg caused a statistically significant body weight reduction compared to vehicle treated mice as early as day 3 after dosing.

The mean maximum weight reduction by C2 antibody was 29.05% by day 14 when the animals were euthanized. 38B8 treated mice had a mean body weight reduction of 15.1167% by day 14. Statistical analysis was done by two-way ANOVA with Bonferroni post-tests.

Mouse Dose Response Determination

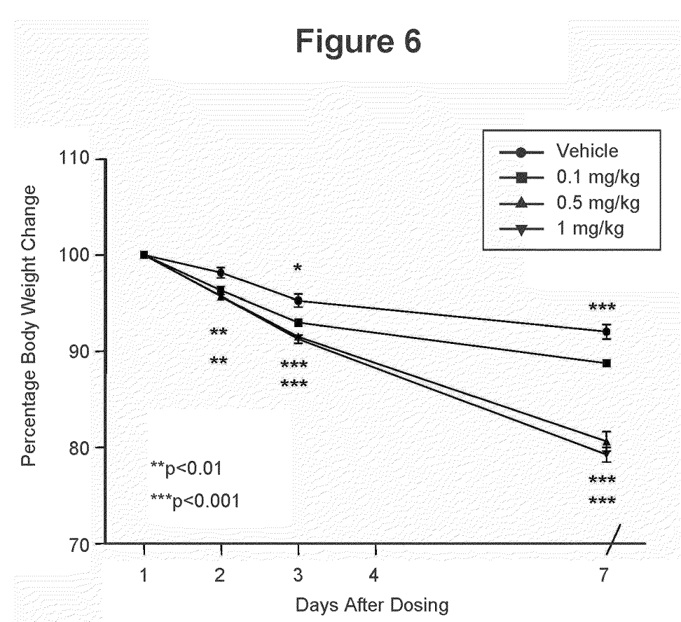

DIO mice were singly-housed and allowed to acclimate for one week. Body weight measurements were taken prior to dosing. To find the lowest effective dose of C2, DIO mice were dosed intraperitoneally with either vehicle, or C2 at 0.1 mg/kg, 0.5 mg/kg, or 1 mg/kg (n=4 for each). Body weight was measured at days 1, 2, 3, 4 and 7 after dosing (FIG. 6).

The results of the dose response study indicate C2 reduces the body weight in a dose dependent manner (FIG. 6). Based on the results, the lowest efficacious dose of C2 for reducing body weight in DIO mice is between 0.5 mg/kg and 0.1 mg/kg. Single injections of C2 at 0.5 mg/kg and 1 mg/kg both yielded similar statistically significant reductions in body weight (FIG. 6). This is comparable to the 2 mg/kg dose of C2 shown in FIG. 5. Statistical analysis was done by two-way ANOVA with Bonferroni post-tests.

In Vivo Antibody Titers

For each antibody, six DIO mice were used to collect samples for pharmacokinetic determination of drug concentration. Each mouse was injected intraperitoneally with 1 mg/kg of antibody. 200 microliters of blood was collected retro-orbitally from two mice at time point 0 and the following times after injection: 15 min, 24 hrs, and 7 days. Two mice were bled for serum collection at the following times after injection: 30 min, 1 hr, 2 days, 8 days, and 11 days. Two mice were bled for serum collection at the following times after injection: 2 hrs, 4 hrs, 3 days, and 9 days. Antibody concentrations were determined by comparison to known concentrations of antibody by ELISA. Plates were coated with recombinant mouse TrkB (R and D systems, Minneapolis, Minn.). A horseradish peroxidase-conjugated secondary specific for mouse IgG Fc was used (Jackson ImmunoResearch Laboratories Inc., West Grove, Pa.). The secondary was detected using a TMB peroxidase detection kit (KPL, Inc., Gaithersburg, Md.).

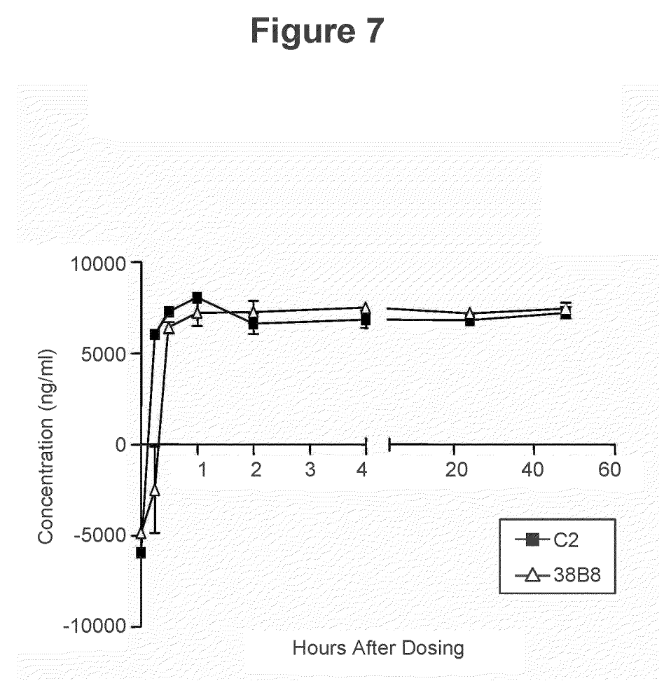

The results show that C2 has a same PK as mouse monoclonal antibody 38B8. Both C2 and 38B8 had high titers of antibody 48 hours after dosing (FIG. 7). The efficacy of each antibody to significantly reduce body weight was maintained up to day 14 after dosing, at which point the animals were euthanized.

Although a body weight reduction is observed in mice treated with agonist anti-TrkB antibody, peripheral injection of agonist anti-TrkB antibody resulted in increased food intake and increased body weight in monkeys. See, U.S. Pat. Appl. Pub. No. 2007/0248611, which is herein incorporated by reference in its entirety.

### Example 7

### Treatment with Agonist Anti-TrkB Antibody Resulted in Increased Survival of Retinal Ganglion Cells (RGCs) in an Animal Model for Glaucoma

This example illustrates the efficacy of agonist anti-TrkB antibody in a mouse glaucoma model. Retinoproprotective efficacy of the agonist anti-TrkB antibody C2 was tested by raising intraocular pressure (10P) in unilateral fashion in albino mice to induce experimental acute glaucoma and following with a single intravitreal injection of either C2 or control mouse IgG1 antibody. In this example, the C2 antibody used was an affinity matured recombinant chimeric antibody with human variable regions (sequences shown in Table 2 above) fused to mouse IgG1 constant region. Survival of retinal ganglion cells (RGCs) in the model was evaluated morphometrically by RGC counting.

Laser-induced transient glaucoma was induced in SJL mice using protocol of Fu and Sretavan (Fu and Sretavan, Invest. Opthalmol. Vis. Sci. 2009). Briefly, 2 month old mice were anesthetized with intraperitoneal injection of ketamine (60 mg/kg) and xylazine (6 mg/kg). Animals were pretreated with 1% atropine (Atropine Sulfate, 1 drop) and tropicamide (1%, 1 drop) eye drops. After loss of toe pinch reflex, mice were placed on the platform of a slit lamp opthalmological microscope equipped with a diode laser (532 nm; OcuLight TX IRIDEX, Mountain View, Calif.). One eye of each animal underwent laser photocoagulation of the episcleral and limbal veins (150 mW laser power, 0.2 second duration, 100 um diameter spot size) to block aqueous outflow. The translimbal laser treatment was performed over 270-300°, sparing the nasal aspect as well as the long posterior ciliary arteries. The contralateral eye was untreated and served as control. To prevent potential infection and to reduce inflammation, bacitracin, neomycin, polymyxin B and hydrocortisone ophthalmic ointment was administered to both eyes. In this treatment IOP is increased only transiently, and the increase is completely resolved in 7 days or less (Fu and Sretavan, Invest Opthalmol V is Sci, 2009).

Intraocular pressure was monitored using rebound tonomentry (TonoLab tonometer, Tiolat Oy, Helsinki, Finland). Tonometry was performed on the animals restrained by intramuscular injection of ketamine (60 mg/kg) and xylazine (6 mg/kg). The first tonometry test was done prior to photocoagulation surgery, and subsequent tonometry tests were done 2 and 5 days after surgery.

Two days after glaucoma induction, drug was administered to the affected eye by intravitreal injection. In brief, animals were anesthetized with ketamine/xylazine injections. Intraocular injections were performed using a micro needles (34 Gauge, World Precision Instrument) connected through adaptor and flexible tubing to a Hamilton syringe mounted on microinjection pump (World Precision Instrument). Substances were injected into the superior aspect of the intravitreal cavity of laser-treated eye, using a trans-scleral approach. 2 μL of 2.34 mg/ml C2 antibody solution or the same quantity of control antibody was injected in the eye (IgG1 produced to non-murine antigen was used as the control antibody). Eyes were checked after injection for vitreous hemorrhage, cataract, and other complications using slit lamp and opthalmological microscope.

Four weeks after glaucoma induction, animals were anesthetized with intraperitoneal injection of avertine (250 mg/kg), perfused with 4% paraformaldehyde fixative and their eyes were processed for histology. Retinas were collected. Surviving RGCs were identified using staining with Brn3a antibody and counted using algorithm developed for ImageJ.

To evaluate neuroprotective activity of C2, surviving RGCs were quantified using immunostaining with Brn3a antibody one month after glaucoma induction. In the retina, Brn3 transcription factors label nuclei of retinal ganglion cells and provide a convenient marker for survival of these neurons.

Animals were anesthetized with intraperitoneal injection of avertine (250 mg/kg), perfused transcardially with 4% paraformaldehyde fixative and their eyes were processed for histology. The anterior segment, lens and optic nerve were dissected away, and the retinas were gently freed from the sclera. The dorsal part of the retina was marked by cutting a notch. Four radial relaxation cuts were made on the retina. Extracted retinas were post-fixed for 30 min in 4% PFA. After fixation, the retinas were rinsed in PBS and transferred to blocking solution (5% bovine serum albumin (BSA), 10% normal goat serum, 0.3% NP40 in PBS), and frozen at −80° overnight. The next day, specimens were thawed at room temperature. Freezing/thawing was used to enhance permeabilization. Tissues were incubated with a primary monoclonal Brn3a antibody (Abcam, 1:100), diluted in the blocking solution for 12 h at 5C. Retinas were washed in PBS three times for 15 minutes each time, and incubated with secondary antibodies (Invitrogen, goat-anti-mouse conjugated with Alexa 488) for 2 hour at room temperature. After stained retinas were washed 3 times in PBS, they were flat-mounted on microscopic slides, photoreceptor side down. Coverslips were mounted over specimens using Prolong Antifade Kit (Invitrogen), and specimens were examined by confocal microscopy.

To evaluate the effect of elevated 10P and drug treatment on RGC survival, 12 0.3 mm² images were collected from each retina. 4 images were collected from central retina, around optic nerve, and 8 images were collected from mid-periphery of retina. Location of images was annotated relative to the retinal orientation. This allowed estimation of densities of retinal ganglion cells in the entire retinas as well as in nasal half of retinas, where RGC loss was most significant, uniformly in all animals. Brn3a-positive RGC nuclei were counted in each frame semi-automatically, using ImageJ software and then averaged for entire retina or for nasal retina.

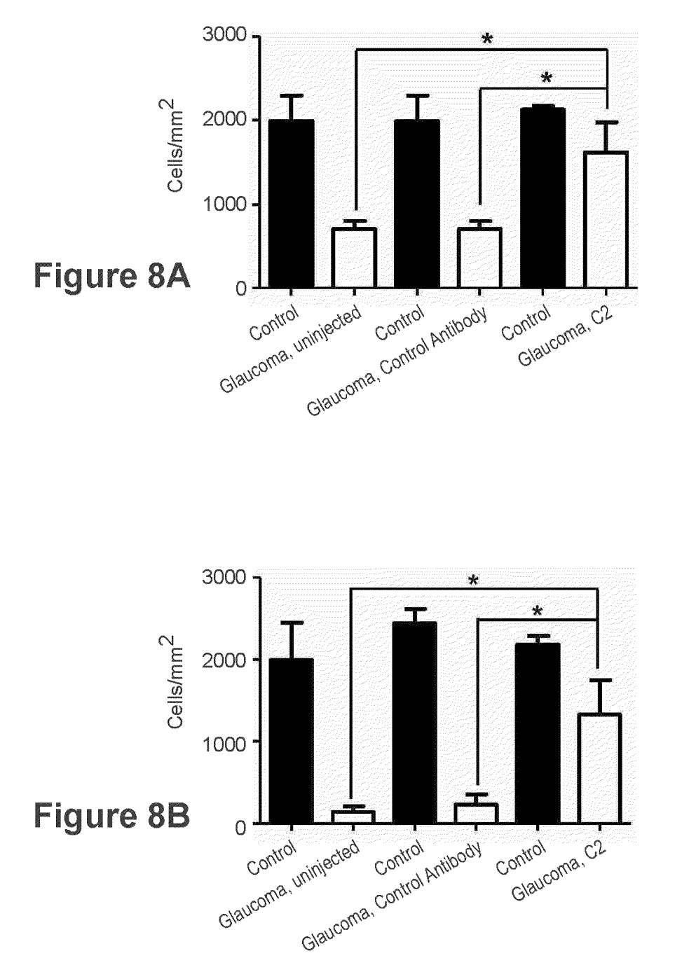

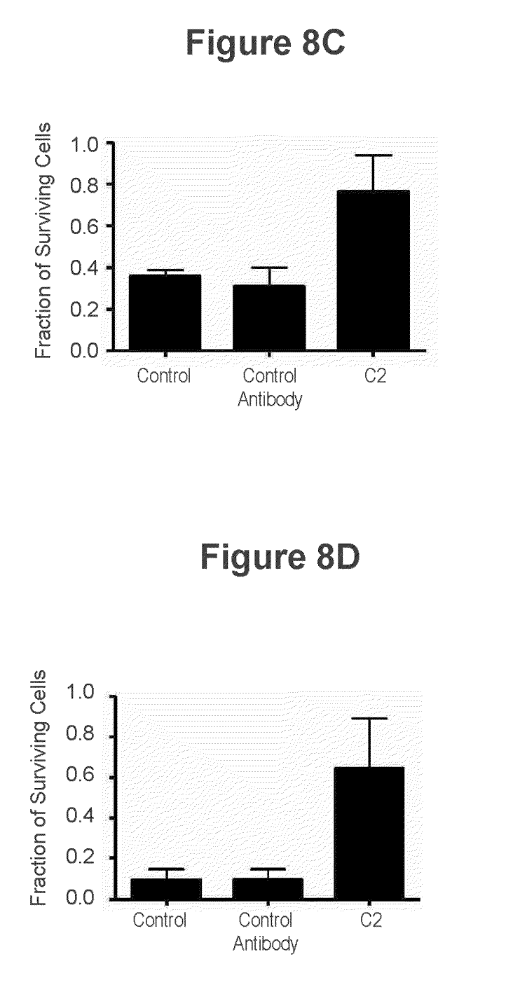

Elevation of intraocular pressure using laser-induced photocoagulation of episcleral and limbal veins resulted in significant loss of RGCs in the retinas from the eyes with elevated intraocular pressure (˜64%, P<0.01), with injury mostly localized to the nasal half of retina (˜90% loss, P<0.01) (FIGS. 8A and 8B). Intraocular injection of control antibody did not affect the extent of damage. However, a single intraocular injection of agonist anti-TrkB antibody resulted in the increased survival of RGCs (76% of surviving cells in the entire retina (FIG. 8C), ˜65% surviving cells in the nasal retina half, P<0.05 (FIG. 8D)). These results demonstrate that intraocular delivery of agonist anti-TrkB antibody can be used as a neuroprotective therapy to treat glaucomatous RGC loss.

Transient short-term elevation of intraocular pressure in the animal model closely simulates the clinical conditions of acute angle closure glaucoma and ischemic retinopathy. The pattern of optic nerve damage in acute angle closure glaucoma is very similar to damage seen in primary open-angle glaucoma, suggesting mechanistic similarities between those two distinct diseases. The data shown above in this example demonstrates that agonist anti-TrkB antibody can protect RGC integrity and health in primary angle closure glaucoma as well as ischemic retinopathy.

Even though IOP elevation in the acute angle closure attack can be successfully treated, damage to the retinal ganglion cells can develop after the IOP resolution. Short IOP attack may create persistent insult that can propagate from damaged neurons to their neighboring neurons as a result of excitotoxicity of neuroinflammation. The relatively short time frame in which nerve damage occurs after angle closure glaucoma attack makes it possible to use neuroprotective intervention applied at the time of surgical treatment and as follow up treatment (Shen, S. Y. et al., Opthalmology, 2006. 113(6): p. 924-9). Therefore, agonist anti-TrkB antibody can be used either alone or in combination with intraocular pressure lowering therapies for the management of acute angle closure glaucoma.

Although the disclosed teachings have been described with reference to various applications, methods, kits, and compositions, it will be appreciated that various changes and modifications can be made without departing from the teachings herein and the claimed invention below. The foregoing examples are provided to better illustrate the disclosed teachings and are not intended to limit the scope of the teachings presented herein. While the present teachings have been described in terms of these exemplary embodiments, the skilled artisan will readily understand that numerous variations and modifications of these exemplary embodiments are possible without undue experimentation. All such variations and modifications are within the scope of the current teachings.

All references cited herein, including patents, patent applications, papers, text books, and the like, and the references cited therein, to the extent that they are not already, are hereby incorporated by reference in their entirety. In the event that one or more of the incorporated literature and similar materials differs from or contradicts this application, including but not limited to defined terms, term usage, described techniques, or the like, this application controls.

The foregoing description and Examples detail certain specific embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof.

It is claimed:

1. An isolated antibody which specifically binds to TrkB, wherein said antibody is capable of promoting neuron survival and comprises:
a heavy chain variable region (VH) complementary determining region one (CDR1) having the amino acid sequence GYTFTNYX¹IX² (SEQ ID NO: 159), wherein X¹ is D or V, and X² is I or L;
a VH complementary determining region two (CDR2) having the amino acid sequence X¹IX²X³X⁴X⁵X⁶X⁷X⁸X⁹X¹⁰X¹¹X¹²KFKX¹³ (SEQ ID NO: 165), wherein X¹ is Y or H, X² is N, S or A, X³ is P or A, X⁴ is Y or A, X⁵ is N, Q, V or A, X⁶ is G, R, D, A or E, X⁷ is R or G, X⁸ is R, T, K or I, X⁹ is E or K, X¹⁰ is Y, E or A, X¹¹ is N or A, X¹² is E or A, and X¹³ is G or Y; and/or
a VH complementary determining region three (CDR3) having the amino acid sequence LLX¹X²X³X⁴X⁵X⁶X⁷X⁸AX⁹X¹⁰X¹¹ (SEQ ID NO: 166), wherein X¹ is K, A or R, X² is Y or A, X³ is R or A, X⁴ is R, C, A or P, X⁵ is F, E, A, H, M or L, X⁶ is R, S, A, K, T or Q, X⁷ is Y, E, A or F, X⁸ is Y, E or A, X⁹ is I, E or A, X¹⁰ is D or H, and X¹¹ is Y, E or V.

2. The antibody of claim 1, wherein said VH CDR2 has the amino acid sequence YINPYNX¹X²X³X⁴YNEKFKG (SEQ ID NO: 160), wherein X¹ is G, R or D, X² is R or G, X³ is R or T, and X⁴ is E or K; and said VH CDR3 has the amino acid sequence LLKYRRFXYYAIDY (SEQ ID NO: 161), wherein X is R or S.

3. The antibody of claim 1, wherein said VH CDR1 has the amino acid sequence shown in SEQ ID NO: 24, said VH CDR2 has the amino acid sequence shown in SEQ ID NO: 25, and said VH CDR3 has the amino acid sequence shown in SEQ ID NO: 3.

4. The antibody of claim 1, wherein the antibody further comprises a light chain variable region (VL) CDR1 having the amino acid sequence shown in SEQ ID NO: 19, a VL CDR2 having the amino acid sequence shown in SEQ ID NO: 5, and a VL CDR3 having the amino acid sequence shown in SEQ ID NO: 6.

5. The antibody of claim 1, wherein the antibody comprises a heavy chain variable region (VH) complementary determining region one (CDR1) having the amino acid sequence shown in SEQ ID NO: 24, a VH CDR2 having the amino acid sequence shown in SEQ ID NO: 25, a VH CDR3 having the amino acid sequence shown in SEQ ID NO: 3, a light chain variable region (VL) CDR1 having the amino acid sequence shown in SEQ ID NO: 19, a VL CDR2 having the amino acid sequence shown in SEQ ID NO: 5, and a VL CDR3 having the amino acid sequence shown in SEQ ID NO: 6.

6. The antibody of claim 5, wherein the VH region comprises the amino acid sequence shown in SEQ ID NO: 12 and the VL region comprises the amino acid sequence shown in SEQ ID NO: 173.

7. The antibody of claim 5, wherein the antibody comprises a light chain having the amino acid sequence shown in SEQ ID NO: 174 and a heavy chain having the amino acid sequence shown in SEQ ID NO: 175, with or without the C-terminal lysine of SEQ ID NO: 175.

8. The antibody of claim 5, wherein the antibody consists of a light chain having the amino acid sequence shown in SEQ ID NO: 174; a heavy chain having the amino acid sequence shown in SEQ ID NO: 175, with or without the C-terminal lysine of SEQ ID NO: 175; or both a light chain having the amino acid sequence shown in SEQ ID NO: 174 and a heavy chain having the amino acid sequence shown in SEQ ID NO: 175, with or without the C-terminal lysine of SEQ ID NO: 175.

9. A pharmaceutical composition comprising a therapeutically effective amount of the antibody of claim 1.

10. A host cell that recombinantly produces the antibody of claim 1.

11. An isolated nucleic acid encoding the antibody of claim 1.

12. A method for treating cachexia in a primate comprising peripherally administering the antibody of claim 1 to a primate suffering from cachexia, thereby ameliorating one or more symptoms of cachexia.

13. A method for treating Charcot-Marie-Tooth (CMT) disease in a patient, comprising administering to the patient a therapeutically effective amount of a TrkB agonist to a patient suffering from CMT disease, thereby ameliorating one or more symptoms of CMT disease.

14. The method of claim 13, wherein the TrkB agonist is an agonist anti-TrkB monoclonal antibody.

15. The method of claim 14, wherein the agonist anti-TrkB monoclonal antibody is the antibody of claim 1.

16. The method of claim 13, wherein said CMT disease is selected from the group consisting of CMT1, CMT2, CMT3, CMT4 and CMTX.

17. The method of claim 16, wherein said CMT1 is selected from the group consisting of CMT1A, CMT1B and CMT1C.

18. The method of claim 13, further comprising administering to the subject a therapeutically effective amount of a TrkC agonist antibody.

19. The method of claim 13, wherein muscle strength and/or compound muscle action potential (CMAP) area is increased following administration of the TrkB agonist.

20. A method for treating and/or preventing glaucoma and/or ischemic retinopathy in an individual, comprising administering a therapeutically effective amount of an agonist anti-TrkB monoclonal antibody to an individual suffering from glaucoma and/or ischemic retinopathy, thereby ameliorating one or more symptoms of glaucoma and/or ischemic retinopathy.


SEQUENCE LISTING (178 sequences)

SEQ ID NO: 1 - protein, 10 aa, Artificial Sequence
  humanized variable region sequence
GYTFTNYDII

SEQ ID NO: 2 - protein, 17 aa, Artificial Sequence
  humanized variable region sequence
YINPYNRRREYNEKFKG

SEQ ID NO: 3 - protein, 14 aa, Artificial Sequence
  humanized variable region sequence
LLKYRRFRYYAIDY

SEQ ID NO: 4 - protein, 11 aa, Artificial Sequence
  humanized variable region sequence
RASENVYSNLA

SEQ ID NO: 5 - protein, 7 aa, Artificial Sequence
  humanized variable region sequence
AASNLQS

SEQ ID NO: 6 - protein, 9 aa, Artificial Sequence
  humanized variable region sequence
QHFWGSPFT

SEQ ID NO: 7 - protein, 107 aa, Artificial Sequence
  humanized variable region sequence
DIQMTQSPSSLSASVGDRVTITCRASENVYSNLAWYQQKPGKAPKLLIYAASNLQSGVPSRFSGSGSGTDFTFTISSLQPEDIATYYCQHFWGSPFTFGQGTKLEIK

SEQ ID NO: 8 - protein, 123 aa, Artificial Sequence
  humanized variable region sequence
QVQLVQSGAEVKKPGASVKVSCKASGYTFTNYDIIWVRQAPGQGLEWMGYINPYNRRREYNEKFKGRVTMTRDTSTSTVYMELSSLRSEDTAVYYCARLLKYRRFRYYAIDYWGQGTTVTVSS

SEQ ID NO: 9 - protein, 107 aa, Artificial Sequence
  humanized variable region sequence
DIQMTQSPSSLSASVGDRVTITCRASENVYSNLAWYQQKPGKAPKLLIYAASNLADGVPSRFSGSGSGTDFTFTISSLQPEDIATYYCQHFWYSPFTFGQGTKLEIK

SEQ ID NO: 10 - protein, 123 aa, Artificial Sequence
  humanized variable region sequence
QVQLVQSGAEVKKPGASVKVSCKASGYTFTNYDIIWVRQAPGQGLEWMGYINPYNDGTKYNEKFKGRVTMTRDTSTSTVYMELSSLRSEDTAVYYCARLLKYRRFSYYAIDYWGQGTTVTVSS

SEQ ID NO: 11 - protein, 107 aa, Artificial Sequence
  humanized variable region sequence
DIQMTQSPSSLSASVGDRVTITCRTSENVYSNLAWYQQKPGKAPKLLIYAASNLQSGVPSRFSGSGSGTDFTFTISSLQPEDIATYYCQHFWGSPFTFGQGTKLEIK

SEQ ID NO: 12 - protein, 123 aa, Artificial Sequence
  humanized variable region sequence
QVQLVQSGAEVKKPGASVKVSCKASGYTFTNYDIIWVRQAPGQGLEWMGYINPYNDGTKYNEKFKGRVTMTRDTSTSTVYMELSSLRSEDTAVYYCARLLKYRRFRYYAIDYWGQGTTVTVSS

SEQ ID NO: 13 - protein, 107 aa, Artificial Sequence
  humanized variable region sequence
DIQMTQSPSSLSASVGDRVTITCHASENVYSNLAWYQQKPGKAPKLLIYAASNLQSGVPSRFSGSGSGTDYTFTISSLQPEDIATYYCQHFWGSPFTFGQGTKLEIK

SEQ ID NO: 14 - protein, 107 aa, Artificial Sequence
  humanized variable region sequence
DIQMTQSPSSLSASVGDRVTITCRASENVYSNLAWYQQKPGKAPKLLIYAASNLQSGVPSRFSGSGSGTDYTFTISSLQPEDIATYYCQHFWGSPFTFGQGTKLEIK

SEQ ID NO: 15 - protein, 107 aa, Artificial Sequence
  humanized variable region sequence
DIQMTQSPSSLSASVGDRVTITCRASEPVYSNVAWYQQKPGKAPKLLIYAASNLQSGVPSRFSGSGSGTDYTFTISSLQPEDIATYYCQHFWGSPFTFGQGTKLEIK

SEQ ID NO: 16 - protein, 123 aa, Artificial Sequence
  humanized variable region sequence
QVQLVQSGAEVKKPGASVKVSCKASGYTFTNYDIIWVRQAPGQGLEWMGYINPYNGRREYNEKFKGRVTMTRDTSTSTVYMELSSLRSEDTAVYYCARLLKYRRFRYYAIDYWGQGTTVTVSS

SEQ ID NO: 17 - protein, 7 aa, Artificial Sequence
  humanized variable region sequence
AASNLAD

SEQ ID NO: 18 - protein, 9 aa, Artificial Sequence
  humanized variable region sequence
QHFWYSPFT

SEQ ID NO: 19 - protein, 11 aa, Artificial Sequence
  humanized variable region sequence
RTSENVYSNLA

SEQ ID NO: 20 - protein, 107 aa, Artificial Sequence
  humanized variable region sequence
DIQMTQSPSSLSASVGDRVTITCXXSXXVXXXXAWYQQKPGKAPKLLIYXASNLXXGVPSRFSGSGSGTDXTFTISSLQPEDIATYYCQXFXXXXXXFGQGTKLEIK

SEQ ID NO: 21 - protein, 123 aa, Artificial Sequence
  humanized variable region sequence
QVQLVQSGAEVKKPGASVKVSCKASGYTFTNYXIXWVRQAPGQGLEWMGXIXXXXXXXXXXXKFKXRVTMTRDTSTSTVYMELSSLRSEDTAVYYCARLLXXXXXXXXAXXXWGQGTTVTVSS

SEQ ID NO: 22 - protein, 11 aa, Artificial Sequence
  humanized variable region sequence
HASENVYSNLA

SEQ ID NO: 23 - protein, 11 aa, Artificial Sequence
  humanized variable region sequence
RASEPVYSNVA

SEQ ID NO: 24 - protein, 5 aa, Artificial Sequence
  humanized variable region sequence
NYDII

SEQ ID NO: 25 - protein, 6 aa, Artificial Sequence
  humanized variable region sequence
PYNDGT

SEQ ID NO: 26 - protein, 14 aa, Artificial Sequence
  humanized variable region sequence
LLKYRRFSYYAIDY

SEQ ID NO: 27 - protein, 6 aa, Artificial Sequence
  humanized variable region sequence
PYNRRR

SEQ ID NO: 28 - protein, 6 aa, Artificial Sequence
  humanized variable region sequence
PYNGRR

SEQ ID NO: 29 - protein, 123 aa, Artificial Sequence
  humanized variable region sequence
QVQLVQSGAEVKKPGASVKVSCKASGYTFTNYDIIWVRQAPGQGLEWMGYINPYQGRREYNEKFKGRVTMTRDTSTSTVYMELSSLRSEDTAVYYCARLLKYRRFRYYAIDYWGQGTTVTVSS

SEQ ID NO: 30 - protein, 123 aa, Artificial Sequence
  humanized variable region sequence
QVQLVQSGAEVKKPGASVKVSCKASGYTFTNYDIIWVRQAPGQGLEWMGYINPYVGRREYNEKFKGRVTMTRDTSTSTVYMELSSLRSEDTAVYYCARLLKYRRFRYYAIDYWGQGTTVTVSS

SEQ ID NO: 31 - protein, 123 aa, Artificial Sequence
  humanized variable region sequence
QVQLVQSGAEVKKPGASVKVSCKASGYTFTNYDIIWVRQAPGQGLEWMGYINPYNARREYNEKFKGRVTMTRDTSTSTVYMELSSLRSEDTAVYYCARLLKYRRFRYYAIDYWGQGTTVTVSS

SEQ ID NO: 32 - protein, 123 aa, Artificial Sequence
  humanized variable region sequence
QVQLVQSGAEVKKPGASVKVSCKASGYTFTNYDIIWVRQAPGQGLEWMGYINPYNDRREYNEKFKGRVTMTRDTSTSTVYMELSSLRSEDTAVYYCARLLKYRRFRYYAIDYWGQGTTVTVSS

SEQ ID NO: 33 - protein, 123 aa, Artificial Sequence
  humanized variable region sequence
QVQLVQSGAEVKKPGASVKVSCKASGYTFTNYDIIWVRQAPGQGLEWMGYINPYNGGREYNEKFKGRVTMTRDTSTSTVYMELSSLRSEDTAVYYCARLLKYRRFRYYAIDYWGQGTTVTVSS

SEQ ID NO: 34 - protein, 123 aa, Artificial Sequence
  humanized variable region sequence
QVQLVQSGAEVKKPGASVKVSCKASGYTFTNYDIIWVRQAPGQGLEWMGYINPYNGRTEYNEKFKGRVTMTRDTSTSTVYMELSSLRSEDTAVYYCARLLKYRRFRYYAIDYWGQGTTVTVSS

SEQ ID NO: 35 - protein, 123 aa, Artificial Sequence
  humanized variable region sequence
QVQLVQSGAEVKKPGASVKVSCKASGYTFTNYDIIWVRQAPGQGLEWMGYINPYNGRRKYNEKFKGRVTMTRDTSTSTVYMELSSLRSEDTAVYYCARLLKYRRFRYYAIDYWGQGTTVTVSS

SEQ ID NO: 36 - protein, 123 aa, Artificial Sequence
  humanized variable region sequence
QVQLVQSGAEVKKPGASVKVSCKASGYTFTNYDIIWVRQAPGQGLEWMGYINPYNGRREYNAKFKGRVTMTRDTSTSTVYMELSSLRSEDTAVYYCARLLKYRRFRYYAIDYWGQGTTVTVSS

SEQ ID NO: 37 - protein, 123 aa, Artificial Sequence
  humanized variable region sequence
QVQLVQSGAEVKKPGASVKVSCKASGYTFTNYDIIWVRQAPGQGLEWMGYISPYNGRREYNEKFKGRVTMTRDTSTSTVYMELSSLRSEDTAVYYCARLLKYRRFRYYAIDYWGQGTTVTVSS

SEQ ID NO: 38 - protein, 123 aa, Artificial Sequence
  humanized variable region sequence
QVQLVQSGAEVKKPGASVKVSCKASGYTFTNYDIIWVRQAPGQGLEWMGYIAPYNGRREYNEKFKGRVTMTRDTSTSTVYMELSSLRSEDTAVYYCARLLKYRRFRYYAIDYWGQGTTVTVSS

SEQ ID NO: 39 - protein, 123 aa, Artificial Sequence
  humanized variable region sequence
QVQLVQSGAEVKKPGASVKVSCKASGYTFTNYDIIWVRQAPGQGLEWMGYINAYNGRREYNEKFKGRVTMTRDTSTSTVYMELSSLRSEDTAVYYCARLLKYRRFRYYAIDYWGQGTTVTVSS

SEQ ID NO: 40 - protein, 123 aa, Artificial Sequence
  humanized variable region sequence
QVQLVQSGAEVKKPGASVKVSCKASGYTFTNYDIIWVRQAPGQGLEWMGYINPANGRREYNEKFKGRVTMTRDTSTSTVYMELSSLRSEDTAVYYCARLLKYRRFRYYAIDYWGQGTTVTVSS

SEQ ID NO: 41 - protein, 123 aa, Artificial Sequence
  humanized variable region sequence
QVQLVQSGAEVKKPGASVKVSCKASGYTFTNYDIIWVRQAPGQGLEWMGYINPYAGRREYNEKFKGRVTMTRDTSTSTVYMELSSLRSEDTAVYYCARLLKYRRFRYYAIDYWGQGTTVTVSS

SEQ ID NO: 42 - protein, 123 aa, Artificial Sequence
  humanized variable region sequence
QVQLVQSGAEVKKPGASVKVSCKASGYTFTNYDIIWVRQAPGQGLEWMGYINPYNGRREANEKFKGRVTMTRDTSTSTVYMELSSLRSEDTAVYYCARLLKYRRFRYYAIDYWGQGTTVTVSS

SEQ ID NO: 43 - protein, 123 aa, Artificial Sequence
  humanized variable region sequence
QVQLVQSGAEVKKPGASVKVSCKASGYTFTNYDIIWVRQAPGQGLEWMGYINPYNGRREYAEKFKGRVTMTRDTSTSTVYMELSSLRSEDTAVYYCARLLKYRRFRYYAIDYWGQGTTVTVSS

SEQ ID NO: 44 - protein, 123 aa, Artificial Sequence
  humanized variable region sequence
QVQLVQSGAEVKKPGASVKVSCKASGYTFTNYDIIWVRQAPGQGLEWMGYINPYNGRREYNEKFKGRVTMTRDTSTSTVYMELSSLRSEDTAVYYCARLLAYRRFRYYAIDYWGQGTTVTVSS

SEQ ID NO: 45 - protein, 123 aa, Artificial Sequence
  humanized variable region sequence
QVQLVQSGAEVKKPGASVKVSCKASGYTFTNYDIIWVRQAPGQGLEWMGYINPYNGRREYNEKFKGRVTMTRDTSTSTVYMELSSLRSEDTAVYYCARLLKYARFRYYAIDYWGQGTTVTVSS

SEQ ID NO: 46 - protein, 123 aa, Artificial Sequence
  humanized variable region sequence
QVQLVQSGAEVKKPGASVKVSCKASGYTFTNYDIIWVRQAPGQGLEWMGYINPYNGRREYNEKFKGRVTMTRDTSTSTVYMELSSLRSEDTAVYYCARLLKYRAFRYYAIDYWGQGTTVTVSS

SEQ ID NO: 47 - protein, 123 aa, Artificial Sequence
  humanized variable region sequence
QVQLVQSGAEVKKPGASVKVSCKASGYTFTNYDIIWVRQAPGQGLEWMGYINPYNGRREYNEKFKGRVTMTRDTSTSTVYMELSSLRSEDTAVYYCARLLKYRRFSYYAIDYWGQGTTVTVSS

SEQ ID NO: 48 - protein, 123 aa, Artificial Sequence
  humanized variable region sequence
QVQLVQSGAEVKKPGASVKVSCKASGYTFTNYDIIWVRQAPGQGLEWMGYINPYNGRREYNEKFKGRVTMTRDTSTSTVYMELSSLRSEDTAVYYCARLLRYRRFRYYAIDYWGQGTTVTVSS

SEQ ID NO: 49 - protein, 123 aa, Artificial Sequence
  humanized variable region sequence
QVQLVQSGAEVKKPGASVKVSCKASGYTFTNYDIIWVRQAPGQGLEWMGYINPYNGRREYNEKFKGRVTMTRDTSTSTVYMELSSLRSEDTAVYYCARLLKYRRFAYYAIDYWGQGTTVTVSS

SEQ ID NO: 50 - protein, 123 aa, Artificial Sequence
  humanized variable region sequence
QVQLVQSGAEVKKPGASVKVSCKASGYTFTNYDIIWVRQAPGQGLEWMGYINPYNGRREYNEKFKGRVTMTRDTSTSTVYMELSSLRSEDTAVYYCARLLKARRFRYYAIDYWGQGTTVTVSS

SEQ ID NO: 51 - protein, 123 aa, Artificial Sequence
  humanized variable region sequence
QVQLVQSGAEVKKPGASVKVSCKASGYTFTNYDIIWVRQAPGQGLEWMGYINPYNGRREYNEKFKGRVTMTRDTSTSTVYMELSSLRSEDTAVYYCARLLKYRRERYYAIDYWGQGTTVTVSS

SEQ ID NO: 52 - protein, 123 aa, Artificial Sequence
  humanized variable region sequence
QVQLVQSGAEVKKPGASVKVSCKASGYTFTNYDIIWVRQAPGQGLEWMGYINPYNGRREYNEKFKGRVTMTRDTSTSTVYMELSSLRSEDTAVYYCARLLKYRRARYYAIDYWGQGTTVTVSS

SEQ ID NO: 53 - protein, 123 aa, Artificial Sequence
  humanized variable region sequence
QVQLVQSGAEVKKPGASVKVSCKASGYTFTNYDIIWVRQAPGQGLEWMGYINPYNGRREYNEKFKGRVTMTRDTSTSTVYMELSSLRSEDTAVYYCARLLKYRRFRAYAIDYWGQGTTVTVSS

SEQ ID NO: 54 - protein, 123 aa, Artificial Sequence
  humanized variable region sequence
QVQLVQSGAEVKKPGASVKVSCKASGYTFTNYDIIWVRQAPGQGLEWMGYINPYNGRREYNEKFKGRVTMTRDTSTSTVYMELSSLRSEDTAVYYCARLLKYRRFRYAAIDYWGQGTTVTVSS

SEQ ID NO: 55 - protein, 123 aa, Artificial Sequence
  humanized variable region sequence
QVQLVQSGAEVKKPGASVKVSCKASGYTFTNYDIIWVRQAPGQGLEWMGYINPYNDRRRYNEKFKGRVTMTRDTSTSTVYMELSSLRSEDTAVYYCARLLKYRRFRYYAIDYWGQGTTVTVSS

SEQ ID NO: 56 - protein, 123 aa, Artificial Sequence
  humanized variable region sequence
QVQLVQSGAEVKKPGASVKVSCKASGYTFTNYDIIWVRQAPGQGLEWMGYINPYNRGREYNEKFKGRVTMTRDTSTSTVYMELSSLRSEDTAVYYCARLLKYRRFRYYAIDYWGQGTTVTVSS

SEQ ID NO: 57 - protein, 123 aa, Artificial Sequence
  humanized variable region sequence
QVQLVQSGAEVKKPGASVKVSCKASGYTFTNYDIIWVRQAPGQGLEWMGYINPYNRRTEYNEKFKGRVTMTRDTSTSTVYMELSSLRSEDTAVYYCARLLKYRRFRYYAIDYWGQGTTVTVSS

SEQ ID NO: 58 - protein, 123 aa, Artificial Sequence
  humanized variable region sequence
QVQLVQSGAEVKKPGASVKVSCKASGYTFTNYDIIWVRQAPGQGLEWMGYINPYNRRRKYNEKFKGRVTMTRDTSTSTVYMELSSLRSEDTAVYYCARLLKYRRFRYYAIDYWGQGTTVTVSS

SEQ ID NO: 59 - protein, 107 aa, Artificial Sequence
  humanized variable region sequence
DIQMTQSPSSLSASVGDRVTITCRASENVYSNLAWYQQKPGKAPKLLIYAASNLQSGVPSRFSGSGSGTDFTFTISSLQPEDIATYYCQHFWYSPFTFGQGTKLEIK

SEQ ID NO: 60 - protein, 123 aa, Artificial Sequence
  humanized variable region sequence
QVQLVQSGAEVKKPGASVKVSCKASGYTFTNYVILWVRQAPGQGLEWMGYINPYNDGTKYNEKFKGRVTMTRDTSTSTVYMELSSLRSEDTAVYYCARLLKYRRFSYYAIDYWGQGTTVTVSS

SEQ ID NO: 61 - protein, 107 aa, Artificial Sequence
  humanized variable region sequence
DIQMTQSPSSLSASVGDRVTITCRASENVYSNLAWYQQKPGKAPKLLIYAASNLQSGVPSRFSGSGSGTDFTFTISSLQPEDIATYYCQHFWKSPFTFGQGTKLEIK

SEQ ID NO: 62 - protein, 123 aa, Artificial Sequence
  humanized variable region sequence
QVQLVQSGAEVKKPGASVKVSCKASGYTFTNYVILWVRQAPGQGLEWMGYINPYNDGTKYNEKFKGRVTMTRDTSTSTVYMELSSLRSEDTAVYYCARLLKYRRHSYYAIDYWGQGTTVTVSS

SEQ ID NO: 63 - protein, 107 aa, Artificial Sequence
  humanized variable region sequence
DIQMTQSPSSLSASVGDRVTITCRASENVYSNLAWYQQKPGKAPKLLIYAASNLQSGVPSRFSGSGSGTDFTFTISSLQPEDIATYYCQGFWYSPFTFGQGTKLEIK

SEQ ID NO: 64 - protein, 123 aa, Artificial Sequence
  humanized variable region sequence
QVQLVQSGAEVKKPGASVKVSCKASGYTFTNYVILWVRQAPGQGLEWMGYINPYNDGTKYNEKFKGRVTMTRDTSTSTVYMELSSLRSEDTAVYYCARLLKYRRFSFYAIDYWGQGTTVTVSS

SEQ ID NO: 65 - protein, 107 aa, Artificial Sequence
  humanized variable region sequence
DIQMTQSPSSLSASVGDRVTITCRASENVYSNLAWYQQKPGKAPKLLIYAASNLQSGVPSRFSGSGSGTDFTFTISSLQPEDIATYYCQNFWYSPFTFGQGTKLEIK

SEQ ID NO: 66 - protein, 123 aa, Artificial Sequence
  humanized variable region sequence
QVQLVQSGAEVKKPGASVKVSCKASGYTFTNYVILWVRQAPGQGLEWMGYINPYNDGTKYNEKFKGRVTMTRDTSTSTVYMELSSLRSEDTAVYYCARLLKYRRFSYYAIDEWGQGTTVTVSS

SEQ ID NO: 67 - protein, 123 aa, Artificial Sequence
  humanized variable region sequence
QVQLVQSGAEVKKPGASVKVSCKASGYTFTNYVILWVRQAPGQGLEWMGYINPYNDGTKYNEKFKGRVTMTRDTSTSTVYMELSSLRSEDTAVYYCARLLKYRRMSYYAIDYWGQGTTVTVSS

SEQ ID NO: 68 - protein, 123 aa, Artificial Sequence
  humanized variable region sequence
QVQLVQSGAEVKKPGASVKVSCKASGYTFTNYVILWVRQAPGQGLEWMGYINPYNDGTKYNEKFKGRVTMTRDTSTSTVYMELSSLRSEDTAVYYCARLLKYRAFSYYAIDYWGQGTTVTVSS

SEQ ID NO: 69 - protein, 123 aa, Artificial Sequence
  humanized variable region sequence
QVQLVQSGAEVKKPGASVKVSCKASGYTFTNYVILWVRQAPGQGLEWMGYINPYNDGTKYNEKFKGRVTMTRDTSTSTVYMELSSLRSEDTAVYYCARLLKYRRFKYYAIDYWGQGTTVTVSS

SEQ ID NO: 70 - protein, 107 aa, Artificial Sequence
  humanized variable region sequence
DIQMTQSPSSLSASVGDRVTITCRASENVYSNLAWYQQKPGKAPKLLIYAASNLQSGVPSRFSGSGSGTDFTFTISSLQPEDIATYYCQHFWYSPFGFGQGTKLEIK

SEQ ID NO: 71 - protein, 107 aa, Artificial Sequence
  humanized variable region sequence
DIQMTQSPSSLSASVGDRVTITCRASENVYSNLAWYQQKPGKAPKLLIYAASNLQSGVPSRFSGSGSGTDFTFTISSLQPEDIATYYCQHFWWSPFTFGQGTKLEIK

SEQ ID NO: 72 - protein, 123 aa, Artificial Sequence
  humanized variable region sequence
QVQLVQSGAEVKKPGASVKVSCKASGYTFTNYVILWVRQAPGQGLEWMGYINPYNDGTKYNEKFKGRVTMTRDTSTSTVYMELSSLRSEDTAVYYCARLLKYRRLSYYAIDYWGQGTTVTVSS

SEQ ID NO: 73 - protein, 107 aa, Artificial Sequence; identical to SEQ ID NO: 65
  humanized variable region sequence

SEQ ID NO: 74 - protein, 123 aa, Artificial Sequence
  humanized variable region sequence
QVQLVQSGAEVKKPGASVKVSCKASGYTFTNYVILWVRQAPGQGLEWMGYINPYNDGTKYNEKFKGRVTMTRDTSTSTVYMELSSLRSEDTAVYYCARLLKYRRFRYYAIDYWGQGTTVTVSS

SEQ ID NO: 75 - protein, 107 aa, Artificial Sequence
  humanized variable region sequence
DIQMTQSPSSLSASVGDRVTITCRASENVYSNLAWYQQKPGKAPKLLIYAASNLQSGVPSRFSGSGSGTDFTFTISSLQPEDIATYYCQVFWYSPFTFGQGTKLEIK

SEQ ID NO: 76 - protein, 107 aa, Artificial Sequence
  humanized variable region sequence
DIQMTQSPSSLSASVGDRVTITCRASENVYSNLAWYQQKPGKAPKLLIYAASNLQSGVPSRFSGSGSGTDFTFTISSLQPEDIATYYCQHFWYRPFTFGQGTKLEIK

SEQ ID NO: 77 - protein, 107 aa, Artificial Sequence
  humanized variable region sequence
DIQMTQSPSSLSASVGDRVTITCRASENVYSNLAWYQQKPGKAPKLLIYAASNLQSGVPSRFSGSGSGTDFTFTISSLQPEDIATYYCQHFWYSGFTFGQGTKLEIK

SEQ ID NO: 78 - protein, 107 aa, Artificial Sequence
  humanized variable region sequence
DIQMTQSPSSLSASVGDRVTITCRASENVYSNLAWYQQKPGKAPKLLIYAASNLQSGVPSRFSGSGSGTDFTFTISSLQPEDIATYYCQHFSYSPFTFGQGTKLEIK

SEQ ID NO: 79 - protein, 107 aa, Artificial Sequence
  humanized variable region sequence
DIQMTQSPSSLSASVGDRVTITCRASENVYSNLAWYQQKPGKAPKLLIYAASNLQSGVPSRFSGSGSGTDFTFTISSLQPEDIATYYCQHFWYSPFIFGQGTKLEIK

SEQ ID NO: 80 - protein, 107 aa, Artificial Sequence
  humanized variable region sequence
DIQMTQSPSSLSASVGDRVTITCRASEPVYSNLAWYQQKPGKAPKLLIYAASNLQSGVPSRFSGSGSGTDYTFTISSLQPEDIATYYCQHFWGSPFTFGQGTKLEIK

SEQ ID NO: 81 - protein, 107 aa, Artificial Sequence
  humanized variable region sequence
DIQMTQSPSSLSASVGDRVTITCRASENVYSNLAWYQQKPGKAPKLLIYAASNLQSGVPSRFSGSGSGTDFTFTISSLQPEDIATYYCQHFWYSPFKFGQGTKLEIK

SEQ ID NO: 82 - protein, 123 aa, Artificial Sequence
  humanized variable region sequence
QVQLVQSGAEVKKPGASVKVSCKASGYTFTNYVILWVRQAPGQGLEWMGYINPYNDGTKYNEKFKGRVTMTRDTSTSTVYMELSSLRSEDTAVYYCARLLKYRRFSYYAEDYWGQGTTVTVSS

SEQ ID NO: 83 - protein, 107 aa, Artificial Sequence
  humanized variable region sequence
DIQMTQSPSSLSASVGDRVTITCRASENVYSNLAWYQQKPGKAPKLLIYAASNLQSGVPSRFSGSGSGTDFTFTISSLQPEDIATYYCQHFWYSPFVFGQGTKLEIK

SEQ ID NO: 84 - protein, 107 aa, Artificial Sequence
  humanized variable region sequence
DIQMTQSPSSLSASVGDRVTITCRASENVYSNLAWYQQKPGKAPKLLIYAASNLQSGVPSRFSGSGSGTDFTFTISSLQPEDIATYYCQHFYYSPFTFGQGTKLEIK

SEQ ID NO: 85 - protein, 107 aa, Artificial Sequence
  humanized variable region sequence
DIQMTQSPSSLSASVGDRVTITCRASENVYRNLAWYQQKPGKAPKLLIYAASNLQSGVPSRFSGSGSGTDYTFTISSLQPEDIATYYCQHFWYSPFTFGQGTKLEIK

SEQ ID NO: 86 - protein, 107 aa, Artificial Sequence
  humanized variable region sequence
DIQMTQSPSSLSASVGDRVTITCRASENVYSNLAWYQQKPGKAPKLLIYAASNLQSGVPSRFSGSGSGTDFTFTISSLQPEDIATYYCQQFWYSPFTFGQGTKLEIK

SEQ ID NO: 87 - protein, 123 aa, Artificial Sequence
  humanized variable region sequence
QVQLVQSGAEVKKPGASVKVSCKASGYTFTNYVILWVRQAPGQGLEWMGYINPYNDGTKYNEKFKGRVTMTRDTSTSTVYMELSSLRSEDTAVYYCARLLKYRRFTYYAIHYWGQGTTVTVSS

SEQ ID NO: 88 - protein, 107 aa, Artificial Sequence
  humanized variable region sequence
DIQMTQSPSSLSASVGDRVTITCRASENVYSNLAWYQQKPGKAPKLLIYAASNLQSGVPSRFSGSGSGTDFTFTISSLQPEDIATYYCQHFDYSPFTFGQGTKLEIK

SEQ ID NO: 89 - protein, 107 aa, Artificial Sequence
  humanized variable region sequence
DIQMTQSPSSLSASVGDRVTITCRASENVYSHLAWYQQKPGKAPKLLIYAASNLQSGVPSRFSGSGSGTDYTFTISSLQPEDIATYYCQHFWGSPFTFGQGTKLEIK

SEQ ID NO: 90 - protein, 123 aa, Artificial Sequence
  humanized variable region sequence
QVQLVQSGAEVKKPGASVKVSCKASGYTFTNYVILWVRQAPGQGLEWMGYINPYNDGTKYNEKFKGRVTMTRDTSTSTVYMELSSLRSEDTAVYYCARLLKYRRFRYYAIDVWGQGTTVTVSS

SEQ ID NO: 91 - protein, 107 aa, Artificial Sequence
  humanized variable region sequence
DIQMTQSPSSLSASVGDRVTITCRASENVYSNLAWYQQKPGKAPKLLIYAASNLQSGVPSRFSGSGSGTDFTFTISSLQPEDIATYYCQHFWYSPFLFGQGTKLEIK

SEQ ID NO: 92 - protein, 123 aa, Artificial Sequence
  humanized variable region sequence
QVQLVQSGAEVKKPGASVKVSCKASGYTFTNYVILWVRQAPGQGLEWMGYINPYNDGTKYNEKFKGRVTMTRDTSTSTVYMELSSLRSEDTAVYYCARLLKYRRFQYYAIDYWGQGTTVTVSS

SEQ ID NO: 93 - protein, 107 aa, Artificial Sequence
  humanized variable region sequence
DIQMTQSPSSLSASVGDRVTITCRASENVYSNLAWYQQKPGKAPKLLIYAASNLQSGVPSRFSGSGSGTDFTFTISSLQPEDIATYYCQHFWQSPFTFGQGTKLEIK

SEQ ID NO: 94 - protein, 107 aa, Artificial Sequence
  humanized variable region sequence
DIQMTQSPSSLSASVGDRVTITCRASENVYSNLAWYQQKPGKAPKLLIYAASNLQSGVPSRFSGSGSGTDFTFTISSLQPEDIATYYCQHFWYSWFTFGQGTKLEIK

SEQ ID NO: 95 - protein, 107 aa, Artificial Sequence
  humanized variable region sequence
DIQMTQSPSSLSASVGDRVTITCRASENVYSNLAWYQQKPGKAPKLLIYAASNLQSGVPSRFSGSGSGTDFTFTISSLQPEDIATYYCQHFWASPFTFGQGTKLEIK

SEQ ID NO: 96 - protein, 107 aa, Artificial Sequence
  humanized variable region sequence
DIQMTQSPSSLSASVGDRVTITCRASENVYSNLAWYQQKPGKAPKLLIYAASNLQSGVPSRFSGSGSGTDFTFTISSLQPEDIATYYCQHFSLSPFTFGQGTKLEIK

SEQ ID NO: 97 - protein, 123 aa, Artificial Sequence
  humanized variable region sequence
QVQLVQSGAEVKKPGASVKVSCKASGYTFTNYVILWVRQAPGQGLEWMGYINPYNDGTKYNEKFKGRVTMTRDTSTSTVYMELSSLRSEDTAVYYCARLLKYRPFSYYAIDYWGQGTTVTVSS

SEQ ID NO: 98 - protein, 107 aa, Artificial Sequence
  humanized variable region sequence
DIQMTQSPSSLSASVGDRVTITCRASENVYSNLAWYQQKPGKAPKLLIYAASNLQSGVPSRFSGSGSGTDFTFTISSLQPEDIATYYCQHFWHSPFTFGQGTKLEIK

SEQ ID NO: 99 - protein, 107 aa, Artificial Sequence
  humanized variable region sequence
DIQMTQSPSSLSASVGDRVTITCRASENVYSNLAWYQQKPGKAPKLLIYAASNLQSGVPSRFSGSGSGTDFTFTISSLQPEDIATYYCQHFWYSPCTFGQGTKLEIK

SEQ ID NO: 100 - protein, 107 aa, Artificial Sequence
  humanized variable region sequence
DIQMTQSPSSLSASVGDRVTITCRASENVYSNLAWYQQKPGKAPKLLIYAASNLQSGVPSRFSGSGSGTDFTFTISSLQPEDIATYYCQHFWYSPWTFGQGTKLEIK

SEQ ID NO: 101 - protein, 123 aa, Artificial Sequence
  humanized variable region sequence
QVQLVQSGAEVKKPGASVKVSCKASGYTFTNYVILWVRQAPGQGLEWMGYINPYNDGTKYNEKFKGRVTMTRDTSTSTVYMELSSLRSEDTAVYYCARLLKYRRFSYYAIDVWGQGTTVTVSS

SEQ ID NO: 102 - protein, 107 aa, Artificial Sequence
  humanized variable region sequence
DIQMTQSPSSLSASVGDRVTITCRASENVYSNLAWYQQKPGKAPKLLIYAASNLQSGVPSRFSGSGSGTDFTFTISSLQPEDIATYYCQHFKYSPFTFGQGTKLEIK

SEQ ID NO: 103 - protein, 107 aa, Artificial Sequence
  humanized variable region sequence
DIQMTQSPSSLSASVGDRVTITCRASENVYSNLAWYQQKPGKAPKLLIYAASNLQSGVPSRFSGSGSGTDFTFTISSLQPEDIATYYCQHFWYLPFTFGQGTKLEIK

SEQ ID NO: 104 - protein, 107 aa, Artificial Sequence
  humanized variable region sequence
DIQMTQSPSSLSASVGDRVTITCRASENVYRNLAWYQQKPGKAPKLLIYAASNLQSGVPSRFSGSGSGTDYTFTISSLQPEDIATYYCQHFWGSPFTFGQGTKLEIK

SEQ ID NO: 105 - protein, 123 aa, Artificial Sequence
  humanized variable region sequence
QVQLVQSGAEVKKPGASVKVSCKASGYTFTNYVILWVRQAPGQGLEWMGYINPYNDGTKYNEKFKGRVTMTRDTSTSTVYMELSSLRSEDTAVYYCARLLKYRRFSYYAADYWGQGTTVTVSS

SEQ ID NO: 106 - protein, 107 aa, Artificial Sequence
  humanized variable region sequence
DIQMTQSPSSLSASVGDRVTITCRASENVYSNLAWYQQKPGKAPKLLIYAASNLQSGVPSRFSGSGSGTDFTFTISSLQPEDIATYYCQHFGYSPFIFGQGTKLEIK

SEQ ID NO: 107 - protein, 123 aa, Artificial Sequence
  humanized variable region sequence
QVQLVQSGAEVKKPGASVKVSCKASGYTFTNYVILWVRQAPGQGLEWMGYINPYNDGTKYNEKFKGRVTMTRDTSTSTVYMELSSLRSEDTAVYYCARLLKYRRFRFYAIDYWGQGTTVTVSS

SEQ ID NO: 108 - protein, 107 aa, Artificial Sequence
  humanized variable region sequence
DIQMTQSPSSLSASVGDRVTITCRASENVYSNLAWYQQKPGKAPKLLIYAASNLQSGVPSRFSGSGSGTDFTFTISSLQPEDIATYYCQHFVYSPFTFGQGTKLEIK

SEQ ID NO: 109 - protein, 107 aa, Artificial Sequence
  humanized variable region sequence
DIQMTQSPSSLSASVGDRVTITCRASENVYSNLAWYQQKPGKAPKLLIYAASNLQSGVPSRFSGSGSGTDFTFTISSLQPEDIATYYCQHFWYSPFAFGQGTKLEIK

SEQ ID NO: 110 - protein, 107 aa, Artificial Sequence
  humanized variable region sequence
DIQMTQSPSSLSASVGDRVTITCRASENVYSNLAWYQQKPGKAPKLLIYAASNLQSGVPSRFSGSGSGTDFTFTISSLQPEDIATYYCQHFWMSPFTFGQGTKLEIK

SEQ ID NO: 111 - protein, 107 aa, Artificial Sequence
  humanized variable region sequence
DIQMTQSPSSLSASVGDRVTITCRASENVYSNLAWYQQKPGKAPKLLIYAASNLQSGVPSRFSGSGSGTDFTFTISSLQPEDIATYYCQHFWYSPFWFGQGTKLEIK

SEQ ID NO: 112 - protein, 107 aa, Artificial Sequence
  humanized variable region sequence
DIQMTQSPSSLSASVGDRVTITCRASETVYSNLAWYQQKPGKAPKLLIYAASNLQSGVPSRFSGSGSGTDFTFTISSLQPEDIATYYCQHFWGSPFTFGQGTKLEIK

SEQ ID NO: 113 - protein, 123 aa, Artificial Sequence; identical to SEQ ID NO: 12
  humanized variable region sequence

SEQ ID NO: 114 - protein, 107 aa, Artificial Sequence
  humanized variable region sequence
DIQMTQSPSSLSASVGDRVTITCRASENVYSHLAWYQQKPGKAPKLLIYAASNLQSGVPSRFSGSGSGTDFTFTISSLQPEDIATYYCQHFWYSPFTFGQGTKLEIK

SEQ ID NO: 115 - protein, 107 aa, Artificial Sequence
  humanized variable region sequence
DIQMTQSPSSLSASVGDRVTITCRASEPVYSNLAWYQQKPGKAPKLLIYAASNLQSGVPSRFSGSGSGTDFTFTISSLQPEDIATYYCQHFWGSPFTFGQGTKLEIK

SEQ ID NO: 116 - protein, 123 aa, Artificial Sequence
  humanized variable region sequence
QVQLVQSGAEVKKPGASVKVSCKASGYTFTNYDIIWVRQAPGQGLEWMGYINPYNDGRKENEKFKGRVTMTRDTSTSTVYMELSSLRSEDTAVYYCARLLKYRRFRYYAIDYWGQGTTVTVSS

SEQ ID NO: 117 - protein, 107 aa, Artificial Sequence
  humanized variable region sequence
DIQMTQSPSSLSASVGDRVTITCRASESVYSNLAWYQQKPGKAPKLLIYAASNLQSGVPSRFSGSGSGTDFTFTISSLQPEDIATYYCQHFVYSPFTFGQGTKLEIK

SEQ ID NO: 118 - protein, 123 aa, Artificial Sequence
  humanized variable region sequence
QVQLVQSGAEVKKPGASVKVSCKASGYTFTNYDIIWVRQAPGQGLEWMGHINPYNGRREYNEKFKGRVTMTRDTSTSTVYMELSSLRSEDTAVYYCARLLKYRRFRYYAIDYWGQGTTVTVSS

SEQ ID NO: 119 - protein, 107 aa, Artificial Sequence
  humanized variable region sequence
DIQMTQSPSSLSASVGDRVTITCRASENVYRNLAWYQQKPGKAPKLLIYAASNLQSGVPSRFSGSGSGTDFTFTISSLQPEDIATYYCQHFVYSPFTFGQGTKLEIK

SEQ ID NO: 120 - protein, 107 aa, Artificial Sequence
  humanized variable region sequence
DIQMTQSPSSLSASVGDRVTITCRASENVYRNLAWYQQKPGKAPKLLIYAASNLQSGVPSRFSGSGSGTDFTFTISSLQPEDIATYYCQHFWYSPFTFGQGTKLEIK

SEQ ID NO: 121 - protein, 107 aa, Artificial Sequence
  humanized variable region sequence
DIQMTQSPSSLSASVGDRVTITCRASENVYSHLAWYQQKPGKAPKLLIYAASNLQSGVPSRFSGSGSGTDFTFTISSLQPEDIATYYCQHFVYSPFTFGQGTKLEIK

SEQ ID NO: 122 - protein, 107 aa, Artificial Sequence
  humanized variable region sequence
DIQMTQSPSSLSASVGDRVTITCRASENVYLNLAWYQQKPGKAPKLLIYAASNLQSGVPSRFSGSGSGTDFTFTISSLQPEDIATYYCQHFWGSPFTFGQGTKLEIK

SEQ ID NO: 123 - protein, 107 aa, Artificial Sequence
  humanized variable region sequence
DIQMTQSPSSLSASVGDRVTITCRASENVYRNLAWYQQKPGKAPKLLIYAASNLQSGVPSRFSGSGSGTDFTFTISSLQPEDIATYYCQHFWGSPFTFGQGTKLEIK

SEQ ID NO: 124 - protein, 107 aa, Artificial Sequence
  humanized variable region sequence
DIQMTQSPSSLSASVGDRVTITCRASEAVYSNLAWYQQKPGKAPKLLIYAASNLQSGVPSRFSGSGSGTDFTFTISSLQPEDIATYYCQHFWGSPFTFGQGTKLEIK

SEQ ID NO: 125 - protein, 107 aa, Artificial Sequence
  humanized variable region sequence
DIQMTQSPSSLSASVGDRVTITCRASENVYRNLAWYQQKPGKAPKLLIYIASNLQSGVPSRFSGSGSGTDFTFTISSLQPEDIATYYCQHFVYSPFTFGQGTKLEIK

SEQ ID NO: 126 - protein, 123 aa, Artificial Sequence
  humanized variable region sequence
QVQLVQSGAEVKKPGASVKVSCKASGYTFTNYDIIWVRQAPGQGLEWMGYINPYNERTEYNEKFKGRVTMTRDTSTSTVYMELSSLRSEDTAVYYCARLLKYRRFRYYAIDYWGQGTTVTVSS

SEQ ID NO: 127 - protein, 107 aa, Artificial Sequence
  humanized variable region sequence
DIQMTQSPSSLSASVGDRVTITCRASTNVYRNLAWYQQKPGKAPKLLIYAASNLQSGVPSRFSGSGSGTDFTFTISSLQPEDIATYYCQHFWGSPFTFGQGTKLEIK

SEQ ID NO: 128 - protein, 107 aa, Artificial Sequence
  humanized variable region sequence
DIQMTQSPSSLSASVGDRVTITCRASSNVYSNLAWYQQKPGKAPKLLIYAASNLQSGVPSRFSGSGSGTDYTFTISSLQPEDIATYYCQHFWGSPFTFGQGTKLEIK

SEQ ID NO: 129 - protein, 107 aa, Artificial Sequence
  humanized variable region sequence
DIQMTQSPSSLSASVGDRVTITCRASENVYSHLAWYQQKPGKAPKLLIYAASNLQSGVPSRFSGSGSGTDFTFTISSLQPEDIATYYCQHFWGSPFTFGQGTKLEIK

SEQ ID NO: 130 - protein, 123 aa, Artificial Sequence
  humanized variable region sequence
QVQLVQSGAEVKKPGASVKVSCKASGYTFTNYDIIWVRQAPGQGLEWMGYINPYNRRIEYNEKFKGRVTMTRDTSTSTVYMELSSLRSEDTAVYYCARLLKYRRFRYYAIDYWGQGTTVTVSS

SEQ ID NO: 131 - protein, 107 aa, Artificial Sequence
  humanized variable region sequence
DIQMTQSPSSLSASVGDRVTITCRASTNVYSNLAWYQQKPGKAPKLLIYAASNLQSGVPSRFSGSGSGTDFTFTISSLQPEDIATYYCQHFWGSPFTFGQGTKLEIK

SEQ ID NO: 132 - protein, 107 aa, Artificial Sequence
  humanized variable region sequence
DIQMTQSPSSLSASVGDRVTITCRASENVYSNLAWYQQKPGKAPKLLIYAASNLQSGVPSRFSGSGSGTDFTFTISSLQPEDIATYYCQHFWDSPFTFGQGTKLEIK

SEQ ID NO: 133 - protein, 107 aa, Artificial Sequence
  humanized variable region sequence
DIQMTQSPSSLSASVGDRVTITCRASKNVYSNLAWYQQKPGKAPKLLIYAASNLQSGVPSRFSGSGSGTDFTFTISSLQPEDIATYYCQHFWGSPFTFGQGTKLEIK

SEQ ID NO: 134 - protein, 107 aa, Artificial Sequence
  humanized variable region sequence
DIQMTQSPSSLSASVGDRVTITCRASKNVYSNLAWYQQKPGKAPKLLIYAASNLQSGVPSRFSGSGSGTDFTFTISSLQPEDIATYYCQQFWYSPFTFGQGTKLEIK

SEQ ID NO: 135 - protein, 107 aa, Artificial Sequence
  humanized variable region sequence
DIQMTQSPSSLSASVGDRVTITCRASENVYSNTAWYQQKPGKAPKLLIYAASNLQSGVPSRFSGSGSGTDYTFTISSLQPEDIATYYCQHFWGSPFTFGQGTKLEIK

SEQ ID NO: 136 - protein, 107 aa, Artificial Sequence
  humanized variable region sequence
DIQMTQSPSSLSASVGDRVTITCRASESVYNNLAWYQQKPGKAPKLLIYAASNLQSGVPSRFSGSGSGTDFTFTISSLQPEDIATYYCQHFWGSPFTFGQGTKLEIK

SEQ ID NO: 137 - protein, 123 aa, Artificial Sequence
  humanized variable region sequence
QVQLVQSGAEVKKPGASVKVSCKASGYTFTNYDIIWVRQAPGQGLEWMGYINPYNDGTKYNEKFKYRVTMTRDTSTSTVYMELSSLRSEDTAVYYCARLLKYRRFRYYAIDYWGQGTTVTVSS

SEQ ID NO: 138 - protein, 107 aa, Artificial Sequence
  humanized variable region sequence
DIQMTQSPSSLSASVGDRVTITCRASEPVYNNLAWYQQKPGKAPKLLIYAASNLQSGVPSRFSGSGSGTDYTFTISSLQPEDIATYYCQHFWYSPFTFGQGTKLEIK

SEQ ID NO: 139 - protein, 107 aa, Artificial Sequence
  humanized variable region sequence
DIQMTQSPSSLSASVGDRVTITCRASKPVYSNLAWYQQKPGKAPKLLIYAASNLQSGVPSRFSGSGSGTDYTFTISSLQPEDIATYYCQHFWGSPFTFGQGTKLEIK

SEQ ID NO: 140 - protein, 107 aa, Artificial Sequence
  humanized variable region sequence
DIQMTQSPSSLSASVGDRVTITCRASENVYNNLAWYQQKPGKAPKLLIYAASNLQSGVPSRFSGSGSGTDYTFTISSLQPEDIATYYCQHFWGSPFTFGQGTKLEIK

SEQ ID NO: 141 - protein, 107 aa, Artificial Sequence
  humanized variable region sequence
DIQMTQSPSSLSASVGDRVTITCRASENVYMNLAWYQQKPGKAPKLLIYAASNLQSGVPSRFSGSGSGTDYTFTISSLQPEDIATYYCQHFWYSPFTFGQGTKLEIK

SEQ ID NO: 142 - protein, 107 aa, Artificial Sequence
  humanized variable region sequence
DIQMTQSPSSLSASVGDRVTITCRASEPVYSNVAWYQQKPGKAPKLLIYAASNLQSGVPSRFSGSGSGTDFTFTISSLQPEDIATYYCQHFWGSPFTFGQGTKLEIK

SEQ ID NO: 143 - protein, 107 aa, Artificial Sequence
  humanized variable region sequence
DIQMTQSPSSLSASVGDRVTITCRASEPVYSNLAWYQQKPGKAPKLLIYAASNLQSGVPSRFSGSGSGTDYTFTISSLQPEDIATYYCQHFWYSPFTFGQGTKLEIK

SEQ ID NO: 144 - protein, 107 aa, Artificial Sequence
  humanized variable region sequence
DIQMTQSPSSLSASVGDRVTITCRASENVTSNLAWYQQKPGKAPKLLIYAASNLQSGVPSRFSGSGSGTDFTFTISSLQPEDIATYYCQHFWGSPFTFGQGTKLEIK

SEQ ID NO: 145 - protein, 107 aa, Artificial Sequence
  humanized variable region sequence
DIQMTQSPSSLSASVGDRVTITCHASENVYSNLAWYQQKPGKAPKLLIYAASNLQSGVPSRFSGSGSGTDFTFTISSLQPEDIATYYCQHFWGSPFTFGQGTKLEIK

SEQ ID NO: 146 - protein, 107 aa, Artificial Sequence
  humanized variable region sequence
DIQMTQSPSSLSASVGDRVTITCRASENVYSHLAWYQQKPGKAPKLLIYAASNLQSGVPSRFSGSGSGTDYTFTISSLQPEDIATYYCQHFVYSPFTFGQGTKLEIK

SEQ ID NO: 147 - protein, 107 aa, Artificial Sequence
  humanized variable region sequence
DIQMTQSPSSLSASVGDRVTITCRASENVYSNLAWYQQKPGKAPKLLIYAASNLQSGVPSRFSGSGSGTDFTFTISSLQPEDIATYYCQHFWESPFTFGQGTKLEIK

SEQ ID NO: 148 - protein, 123 aa, Artificial Sequence
  humanized variable region sequence
QVQLVQSGAEVKKPGASVKVSCKASGYTFTNYDIIWVRQAPGQGLEWMGYINPYNDGTKYNEKFKGRVTMTRDTSTSTVYMELSSLRSEDTAVYYCARLLKYACFREEAIDYWGQGTTVTVSS

SEQ ID NO: 149 - protein, 107 aa, Artificial Sequence
  humanized variable region sequence
DIQMTQSPSSLSASVGDRVTITCRTSENVYSNLAWYQQKPGKAPKLLIYAASNLQSGVPSRFSGSGSGTDFTFTISSLQPEDIATYYCQQFWYSPFTFGQGTKLEIK

SEQ ID NO: 150 - protein, 123 aa, Artificial Sequence
  humanized variable region sequence
QVQLVQSGAEVKKPGASVKVSCKASGYTFTNYDIIWVRQAPGQGLEWMGYINPYNDGTKYNEKFKGRVTMTRDTSTSTVYMELSSLRSEDTAVYYCARLLKYRRFKYYAIDYWGQGTTVTVSS

SEQ ID NO: 151 - protein, 107 aa, Artificial Sequence
  humanized variable region sequence
DIQMTQSPSSLSASVGDRVTITCRASENVYSNLAWYQQKPGKAPKLLIYAASNLADGVPSRFSGSGSGTDFTFTISSLQPEDIATYYCQHFWASPFTFGQGTKLEIK

SEQ ID NO: 152 - protein, 107 aa, Artificial Sequence
  humanized variable region sequence
DIQMTQSPSSLSASVGDRVTITCRTSESVYSNLAWYQQKPGKAPKLLIYAASNLQSGVPSRFSGSGSGTDFTFTISSLQPEDIATYYCQHFWASPFTFGQGTKLEIK

SEQ ID NO: 153 - protein, 107 aa, Artificial Sequence
  humanized variable region sequence
DIQMTQSPSSLSASVGDRVTITCRTSENVYSNLAWYQQKPGKAPKLLIYAASNLQSGVPSRFSGSGSGTDFTFTISSLQPEDIATYYCQHFWYSPWTFGQGTKLEIK

SEQ ID NO: 154 - protein, 107 aa, Artificial Sequence
  humanized variable region sequence
DIQMTQSPSSLSASVGDRVTITCRASENVYSNLAWYQQKPGKAPKLLIYAASNLADGVPSRFSGSGSGTDFTFTISSLQPEDIATYYCQHFWTSPFTFGQGTKLEIK

SEQ ID NO: 155 - protein, 107 aa, Artificial Sequence
  humanized variable region sequence
DIQMTQSPSSLSASVGDRVTITCRASENVYSNLAWYQQKPGKAPKLLIYAASNLADGVPSRFSGSGSGTDFTFTISSLQPEDIATYYCQDFWYSPFTFGQGTKLEIK

SEQ ID NO: 156 - protein, 107 aa, Artificial Sequence
  humanized variable region sequence
DIQMTQSPSSLSASVGDRVTITCRTSENVYSNLAWYQQKPGKAPKLLIYAASNLADGVPSRFSGSGSGTDFTFTISSLQPEDIATYYCQHFWASPFTFGQGTKLEIK

SEQ ID NO: 157 - protein, 107 aa, Artificial Sequence
  humanized variable region sequence
DIQMTQSPSSLSASVGDRVTITCRASENVYRNLAWYQQKPGKAPKLLIYAASNLQSGVPSRFSGSGSGTDYTFTISSLQPEDIATYYCQHFVYSPFTFGQGTKLEIK

SEQ ID NO: 158 - protein, 107 aa, Artificial Sequence
  humanized variable region sequence
DIQMTQSPSSLSASVGDRVTITCRASENVTSNLAWYQQKPGKAPKLLIYAASNLQSGVPSRFSGSGSGTDYTFTISSLQPEDIATYYCQHFWGSPFTFGQGTKLEIK

SEQ ID NO: 159 - protein, 10 aa, Artificial Sequence
  humanized variable region sequence
GYTFTNYXIX

SEQ ID NO: 160 - protein, 17 aa, Artificial Sequence
  humanized variable region sequence
YINPYNXXXXYNEKFKG

SEQ ID NO: 161 - protein, 14 aa, Artificial Sequence
  humanized variable region sequence
LLKYRRFXYYAIDY

SEQ ID NO: 162 - protein, 11 aa, Artificial Sequence
  humanized variable region sequence
XXSEXVYSNXA

SEQ ID NO: 163 - protein, 7 aa, Artificial Sequence
  humanized variable region sequence
AASNLXX

SEQ ID NO: 164 - protein, 9 aa, Artificial Sequence
  humanized variable region sequence
QHFWXSPFT

SEQ ID NO: 165 - protein, 17 aa, Artificial Sequence
  humanized variable region sequence
XIXXXXXXXXXXXKFKX

SEQ ID NO: 166 - protein, 14 aa, Artificial Sequence
  humanized variable region sequence
LLXXXXXXXXAXXX

SEQ ID NO: 167 - protein, 11 aa, Artificial Sequence
  humanized variable region sequence
XXSXXVXXXXA

SEQ ID NO: 168 - protein, 7 aa, Artificial Sequence
  humanized variable region sequence
XASNLXX

SEQ ID NO: 169 - protein, 9 aa, Artificial Sequence
  humanized variable region sequence
QXFXXXXXX

SEQ ID NO: 170 - protein, 107 aa, Artificial Sequence
  humanized variable region sequence
DIQMTQSPSSLSASVGDRVTITCXXSEXVYSNXAWYQQKPGKAPKLLIYAASNLXXGVPSRFSGSGSGTDXTFTISSLQPEDIATYYCQHFWXSPFTFGQGTKLEIK

SEQ ID NO: 171 - protein, 123 aa, Artificial Sequence
  humanized variable region sequence
QVQLVQSGAEVKKPGASVKVSCKASGYTFTNYXIXWVRQAPGQGLEWMGYINPYNXXXXYNEKFKGRVTMTRDTSTSTVYMELSSLRSEDTAVYYCARLLKYRRFXYYAIDYWGQGTTVTVSS

SEQ ID NO: 172 - protein, 15 aa, Artificial Sequence
  linking peptide
GGGGSGGGGSGGGGS

SEQ ID NO: 173 - protein, 107 aa, Artificial Sequence
  humanized variable region sequence
DIQMTQSPSSLSASVGDRVTITCRTSENVYSNLAWYQQKPGKAPKLLIYAASNLQSGVPSRFSGSGSGTDYTFTISSLQPEDIATYYCQHFWGSPFTFGQGTKLEIK

SEQ ID NO: 174 - protein, 213 aa, Artificial Sequence
  C2 humanized light chain sequence
DIQMTQSPSSLSASVGDRVTITCRTSENVYSNLAWYQQKPGKAPKLLIYAASNLQSGVPSRFSGSGSGTDYTFTISSLQPEDIATYYCQHFWGSPFTFGQGTKLEIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 175 - protein, 437 aa, Artificial Sequence [sequence omitted]

SEQ ID NO: 176 - protein, 107 aa, Artificial Sequence
  humanized variable region sequence A2 light chain
DIQMTQSPSSLSASVGDRVTITCRASENVYSNLAWYQQKPGKAPKLLIYAASNLQSGVPSRFSGSGSGTDYTFTISSLQPEDIATYYCQHFWYSPWTFGQGTKLEIK

SEQ ID NO: 177 - protein, 9 aa, Artificial Sequence
  humanized variable region sequence A2 CDR3
QHFWYSPWT

SEQ ID NO: 178 - protein, 213 aa, Artificial Sequence
  A2 humanized light chain sequence
DIQMTQSPSSLSASVGDRVTITCRTSENVYSNLAWYQQKPGKAPKLLIYAASNLQSGVPSRFSGSGSGTDYTFTISSLQPEDIATYYCQHFWYSPWTFGQGTKLEIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC